(19) United States
(12) Patent Application Publication
(10) Pub. No.: US 2017/0143267 A1
(43) Pub. Date: May 25, 2017

(54) USER-SPECIFIC SCALE-BASED ENTERPRISE METHODS AND SYSTEMS

(71) Applicant: Physiowave, Inc., Santa Clara, CA (US)

(72) Inventors: Gregory T. Kovacs, Palo Alto, CA (US); Richard M. Wiard, Campbell, CA (US)

(21) Appl. No.: 15/354,938

(22) Filed: Nov. 17, 2016

Related U.S. Application Data
(63) Continuation of application No. PCT/US2016/062484, filed on Nov. 17, 2016.
Application No. PCT/US2016/062484 is a continuation of application No. PCT/US2016/062505, filed on Nov. 17, 2016.
(60) Provisional application No. 62/258,238, filed on Nov. 20, 2015.
(60) Provisional application No. 62/266,496, filed on Dec. 11, 2015.
(60) Provisional application No. 62/266,523, filed on Dec. 11, 2015.

(51) Int. Cl. A61B 5/00; G06F 19/00; A61B 5/145; A61B 5/0402; A61B 5/0476; A61B 5/01; G01G 19/50; A61B 5/024

(52) U.S. Cl. CPC A61B 5/6887; G01G 19/50; G06F 19/3431; G06F 19/3406; G06F 19/345; A61B 5/7275; A61B 5/742; A61B 5/0015; A61B 5/024; A61B 5/0402; A61B 5/0476; A61B 5/01; A61B 5/14542; A61B 5/0816

(57) ABSTRACT

Certain aspects of the disclosure are directed an apparatus including a weighing scale and external circuitry. The weighing scale includes a platform in which a plurality of electrodes and force sensor circuitry are integrated and processing circuitry. The processing circuitry is configured to collect physiological data from the user while the user is standing on the platform, identify a risk that the user has a health condition using trigger data indicative of risks for a plurality of health conditions, wherein the trigger data includes values of the physiological data that are indicative of the risks for the plurality of health conditions, and output at least portions of the physiological data as user data in response to the identified risk. The external circuitry filters data from the Internet using the user data, and identifies data related to the health condition based on the filter of the data from the Internet.

### RELATED APPLICATION DATA

This application is related to PCT Application (Ser. No. PCT/US2016/062484), entitled “Scale-Based Parameter Acquisition Methods and Apparatuses”, filed on Nov. 17, 2016, PCT Application (Ser. No. PCT/US2016/062505), entitled “Remote Physiologic Parameter Assessment Methods and Platform Apparatuses”, filed on Nov. 17, 2016, U.S. Provisional Application (Ser. No 62/258,238), entitled “Condition or Treatment Assessment Methods and Platform Apparatuses”, filed Nov. 20, 2015, U.S. Provisional Application (Ser. No. 62/266,496), entitled “User-Specific Scale-Based Enterprise System”, filed Dec. 11, 2015, and U.S. Provisional Application (Ser. No. 62/266,523) entitled “Social Grouping Using a User-Specific Scale-Based Enterprise System”, filed Dec. 11, 2015, which are fully incorporated herein by reference.

### OVERVIEW

Various aspects of the present disclosure are directed toward methods, systems and apparatuses that are useful in a user-specific scale-based enterprise system.

Various aspects of the present disclosure are directed to monitoring different physiological characteristics are monitored for many different applications. For instance, physiological monitoring instruments are often used to measure a number of patient vital signs, including blood oxygen level, body temperature, respiration rate and electrical activity for electrocardiogram (ECG) or electroencephalogram (EEG) measurements. For ECG measurements, a number of electrocardiograph leads may be connected to a patient's skin, and are used to obtain a signal from the patient.

Obtaining physiological signals (e.g., data) can often require specialty equipment and intervention with medical professionals. For many applications, such requirements may be costly or burdensome. These and other matters have presented challenges to monitoring physiological characteristics.

Aspects of the present disclosure are directed to a user-specific scale-based enterprise system. The user-specific scale-based enterprise system includes at least one scale, the Internet (e.g., world-wide-web), a standalone user CPU, and one or more user devices, such as a smartwatch, fitness tracking device, smartphone, smartbed, among other devices. The scale collects highly user-sensitive data, such as cardiogram data and data indicative of disorders and disease, and other user data, such as demographic information and weight. The one or more user devices include devices that collect various user-sensitive data, such as exercise data, food intake or liquid intake data, sleep data, cardiogram data, among other information. The standalone user CPU includes a user device that includes processing circuitry and/or a user display that is easier for the user to view data than the scale or other user devices. The standalone user CPU, and other user devices form a robust graphical user interface (R-GUI) for the user to view various data. In some aspects, the standalone user CPU includes a personal computer, a laptop, a tablet, and/or a smartphone.

In specific aspects, the scale acts as a hub for user-sensitive data from various user devices, such as cardio-related data, exercise data, food or liquid tracking data, among other data. In further specific embodiments, the scale includes trigger data that triggers a filter data on the enterprise system, include user data and data from the Internet. In various specific embodiments, the enterprise system filters the user data for data correlated with the condition and filters the Internet for various data regarding the condition and/or matching the filtered user data. Thereby, the scale-based enterprise system is used as a medical analytic driver that filters the Internet based on user data related to a condition and trigger data

Various aspects of the present disclosure are directed toward multisensory biometric devices, systems and methods. Aspects of the present disclosure include user-interactive platforms, such as scales, large and/or full platform-area or dominating-area displays and related weighing devices, systems, and methods. Additionally, the present disclosure relates to electronic body scales that use impedance-based biometric measurements. Various other aspects of the present disclosure are directed to biometrics measurements such as body composition and cardiovascular information. Impedance measurements are made through the feet to measure fat percentage, muscle mass percentage and body water percentage. Additionally, foot impedance-based cardiovascular measurements are made for an ECG and sensing the properties of blood pulsations in the arteries, also known as impedance plethysmography (IPG), where both techniques are used to quantify heart rate and/or pulse arrival timings (PAT). Cardiovascular IPG measures the change in impedance through the corresponding arteries between the sensing electrode pair segments synchronous to each heartbeat.

In certain aspects, the present disclosure is directed to apparatuses and methods including a scale and external circuitry. The scale is configured to collect signals from a plurality of users and associate the respective collected signals with a user among the plurality using a scale-based biometric. The scale includes a user display to display data to a user while the user is standing on the scale, a platform for a user to stand on, and processing circuitry. The scale further can include data-procurement circuitry that includes force sensor circuitry and a plurality of electrodes integrated with the platform for engaging the user with electrical signals and collecting signals indicative of the user's identity and cardio-physiological measurements while the user is standing on the platform. The processing circuitry includes a CPU and a memory circuit with user corresponding data stored in the memory circuit. The processing circuitry is arranged with (e.g., electrically integrated with or otherwise in communication) with the force sensor circuitry and the plurality of electrodes and configured to process data obtained by the data-procurement circuitry while the user is standing on the platform and therefrom generate cardio-related physiologic data corresponding to the collected signals. For example, the processing circuitry collects physiological data from the user while the user is standing on the platform, and identifies the user based on an identified scale-based biometric within the physiological data. The processing circuitry can further identify a risk that the user has a health condition using trigger data indicative of risks for a plurality of health conditions, wherein the trigger data includes values of the physiological data that are indicative of the risks for the plurality of health conditions, and output at least portions of the physiological data as user data in response to the identified risk to external circuitry.

The external circuitry includes processing circuitry and memory circuitry. The external circuitry receive the user data from the scale, and in response filter data from the Internet using the user data. Further, the external circuitry can identify data related to the health condition based on the filter of the data from the Internet.

In certain embodiments, aspects as described herein are implemented in accordance with and/or in combination with aspects of the PCT Application (Ser. No. PCT/US2016/062484), entitled “Scale-Based Parameter Acquisition Methods and Apparatuses”, filed on Nov. 17, 2016, PCT Application (Ser. No. PCT/US2016/062505), entitled “Remote Physiologic Parameter Assessment Methods and Platform Apparatuses”, filed on Nov. 17, 2016, U.S. Provisional Application (Ser. No 62/258,238), entitled “Condition or Treatment Assessment Methods and Platform Apparatuses”, filed Nov. 20, 2015, U.S. Provisional Application (Ser. No. 62/266,496), entitled “User-Specific Scale-Based Enterprise System”, filed Dec. 11, 2015, and U.S. Provisional Application (Ser. No. 62/266,523) entitled “Social Grouping Using a User-Specific Scale-Based Enterprise System”, filed Dec. 11, 2015, which are fully incorporated herein by reference.

The above discussion/summary is not intended to describe each embodiment or every implementation of the present disclosure. The figures and detailed description that follow also exemplify various embodiments.

### BRIEF DESCRIPTION OF THE DRAWINGS

Various example embodiments may be more completely understood in consideration of the following detailed description in connection with the accompanying drawings, in which:

FIG. 1a shows an apparatus consistent with aspects of the present disclosure;

FIG. 1b shows an example of a user-specific scale-based enterprise system consistent with aspects of the present disclosure;

FIG. 1c shows an example of a scale aggregating and communicating user data from various user devices, consistent with aspects of the present disclosure;

FIG. 1d shows an example of filtering data from a user-specific scale-based enterprise system, consistent with aspects of the present disclosure;

FIG. 1e shows current paths through the body for the IPG trigger pulse and Foot IPG, consistent with various aspects of the present disclosure;

FIG. 1f is a flow chart illustrating an example manner in which a user-specific physiologic meter/scale may be programmed to provide features consistent with aspects of the present disclosure;

FIG. 2a shows an example of the insensitivity to foot placement on scale electrodes with multiple excitation and sensing current paths, consistent with various aspects of the present disclosure;

FIGS. 2b-2c show examples of electrode configurations, consistent with various aspects of the disclosure;

FIGS. 3a-3b show example block diagrams depicting circuitry for sensing and measuring the cardiovascular time-varying IPG raw signals and steps to obtain a filtered IPG waveform, consistent with various aspects of the present disclosure;

FIG. 3c depicts an example block diagram of circuitry for operating core circuits and modules, including for example those of FIGS. 3a-3b, used in various specific embodiments of the present disclosure;

FIG. 3d shows an exemplary block diagram depicting the circuitry for interpreting signals received from electrodes;

FIG. 4 shows an example block diagram depicting signal processing steps to obtain fiducial references from the individual Leg IPG “beats,” which are subsequently used to obtain fiducials in the Foot IPG, consistent with various aspects of the present disclosure;

FIG. 5 shows an example flowchart depicting signal processing to segment individual Foot IPG “beats” to produce an averaged IPG waveform of improved SNR, which is subsequently used to determine the fiducial of the averaged Foot IPG, consistent with various aspects of the present disclosure;

FIG. 6a shows examples of the Leg IPG signal with fiducials; the segmented Leg IPG into beats; and the ensemble-averaged Leg IPG beat with fiducials and calculated SNR, for an exemplary high-quality recording, consistent with various aspects of the present disclosure;

FIG. 6b shows examples of the Foot IPG signal with fiducials derived from the Leg IPG fiducials; the segmented Foot IPG into beats; and the ensemble-averaged Foot IPG beat with fiducials and calculated SNR, for an exemplary high-quality recording, consistent with various aspects of the present disclosure;

FIG. 7a shows examples of the Leg IPG signal with fiducials; the segmented Leg IPG into beats; and the ensemble-averaged Leg IPG beat with fiducials and calculated SNR, for an exemplary low-quality recording, consistent with various aspects of the present disclosure;

FIG. 7b shows examples of the Foot IPG signal with fiducials derived from the Leg IPG fiducials; the segmented Foot IPG into beats; and the ensemble-averaged Foot IPG beat with fiducials and calculated SNR, for an exemplary low-quality recording, consistent with various aspects of the present disclosure;

FIG. 8 shows an example correlation plot for the reliability in obtaining the low SNR Foot IPG pulse for a 30-second recording, using the first impedance signal as the trigger pulse, from a study including 61 test subjects with various heart rates, consistent with various aspects of the present disclosure;

FIGS. 9a-b show an example configuration to obtain the pulse transit time (PTT), using the first IPG as the triggering pulse for the Foot IPG and ballistocardiogram (BCG), consistent with various aspects of the present disclosure;

FIG. 10 shows nomenclature and relationships of various cardiovascular timings, consistent with various aspects of the present disclosure;

FIG. 11 shows an example graph of PTT correlations for two detection methods (white dots) Foot IPG only, and (black dots) Dual-IPG method, consistent with various aspects of the present disclosure;

FIG. 12 shows an example graph of pulse wave velocity (PWV) obtained from the present disclosure compared to the ages of 61 human test subjects, consistent with various aspects of the present disclosure;

FIG. 13 shows another example of a scale with interleaved foot electrodes to inject and sense current from one foot to another foot, and within one foot, consistent with various aspects of the present disclosure;

FIG. 14a shows another example of a scale with interleaved foot electrodes to inject and sense current from one foot to another foot, and measure Foot IPG signals in both feet, consistent with various aspects of the present disclosure;

FIG. 14b shows another example of a scale with interleaved foot electrodes to inject and sense current from one foot to another foot, and measure Foot IPG signals in both feet, consistent with various aspects of the present disclosure;

FIG. 14c shows another example approach to floating current sources is the use of transformer-coupled current sources, consistent with various aspects of the present disclosure;

FIGS. 15a-d show an example breakdown of a scale with interleaved foot electrodes to inject and sense current from one foot to another foot, and within one foot, consistent with various aspects of the present disclosure;

FIG. 16 shows an example block diagram of circuit-based building blocks, consistent with various aspects of the present disclosure;

FIG. 17 shows an example flow diagram, consistent with various aspects of the present disclosure;

FIG. 18 shows an example scale communicatively coupled to a wireless device, consistent with various aspects of the present disclosure; and

FIGS. 19a-c show example impedance as measured through different parts of the foot based on the foot position, consistent with various aspects of the present disclosure.

While various embodiments discussed herein are amenable to modifications and alternative forms, aspects thereof have been shown by way of example in the drawings and will be described in detail. It should be understood, however, that the intention is not to limit the disclosure to the particular embodiments described. On the contrary, the intention is to cover all modifications, equivalents, and alternatives falling within the scope of the disclosure including aspects defined in the claims. In addition, the term “example” as used throughout this application is only by way of illustration, and not limitation.

### DESCRIPTION

Aspects of the present disclosure are believed to be applicable to a variety of different types of apparatuses, systems, and methods involving a user-specific scale-based enterprise system. In certain implementations, aspects of the present disclosure have been shown to be beneficial when used in the context of a user-specific scale-based enterprise system, including a weighing scale, one or more other user devices, a standalone user CPU, and the world-wide-web. In specific embodiments, the scale acts as a hub for user-sensitive data from various user devices, such as cardio-related data, exercise data, food or liquid tracking data, among other data. In further specific embodiments, the scale includes trigger data that triggers a filter data on the enterprise system, include user data and data from the Internet. In various specific embodiments, the enterprise system filters the user data for data correlated with the condition and filters the Internet for various data regarding the condition and/or matching the filtered user data. Thereby, the scale-based enterprise system is used as a medical analytic driver that filters the Internet based on user data related to a condition and trigger data. These and other aspects can be implemented to address challenges, including those discussed in the background above. While not necessarily so limited, various aspects may be appreciated through a discussion of examples using such exemplary contexts.

Accordingly, in the following description various specific details are set forth to describe specific examples presented herein. It should be apparent to one skilled in the art, however, that one or more other examples and/or variations of these examples may be practiced without all the specific details given below. In other instances, well known features have not been described in detail so as not to obscure the description of the examples herein. For ease of illustration, the same reference numerals may be used in different diagrams to refer to the same elements or additional instances of the same element. Also, although aspects and features may in some cases be described in individual figures, it will be appreciated that features from one figure or embodiment can be combined with features of another figure or embodiment even though the combination is not explicitly shown or explicitly described as a combination.

Embodiments of the present disclosure are directed to a user-specific scale-based enterprise system. The user-specific scale-based enterprise system includes at least one scale, the Internet (e.g., world-wide-web), a standalone user CPU, and one or more user devices, such as a smartwatch, fitness tracking device, smartphone, smartbed, among other devices. The scale collects highly user-sensitive data, such as cardiogram data and data indicative of disorders and disease, and other user data, such as demographic information and weight. The one or more user devices include devices that collect various user-sensitive data, such as exercise data, food intake or liquid intake data, sleep data, cardiogram data, among other information. The standalone user CPU includes a user device that includes processing circuitry and/or a user display that is easier for the user to view data than the scale or other user devices. The standalone user CPU, and other user devices form a robust graphical user interface (R-GUI) for the user to view various data. In some embodiments, the standalone user CPU includes a personal computer, a laptop, a tablet, and/or a smartphone.

In various embodiments, the scale includes trigger data. The trigger data includes user data values and/or combinations of different data values with user demographic information that indicates that the user has a risk for a condition, such as a disorder or disease. In response to the trigger data and the scale-obtained data or other user data from the other devices indicating that the user has a risk for a condition, the scale and/or standalone user CPU filters the user data from the scale and the other user devices and filters data from the Internet to identify data that is relevant to the condition. In this manner, the enterprise system is used as a medical analytic driver that filters scale-obtained data, user device-obtained data, and data from the Internet to identify data related to the condition.

In various embodiments, in response to the filters, the system provides the user with various additional health information regarding the condition. Alternatively and/or in addition, the system provides a prompt to the user that indicates general information about the condition if the user is indicating some risk for the condition. The prompt asks if the user would like more information and in response to the user requesting more information, the enterprise system provides the aggregated user data to a physician for review and to confirm the diagnosis. The physician is provided access to the user data using the internet and/or external circuitry, such as server CPU that is accessible by the physician. In response to the physician confirming the diagnosis and/or correlation, the scale is modified with the confirmed diagnosis. The modification, in some embodiments, includes storing, on the scale, various correlation data (e.g., diagnosis data), adding additional devices and/or parameters to track (e.g., halter monitor, ECG tracking device, prescription drug titration, weight tracking and/or threshold values, exercise goals, stress test), and/or health information about the condition (e.g., articles), among other data. Furthermore, the standalone user CPU of the enterprise system, in some embodiments, is used to display various data to the user, such as generic health information, user-specific diagnosis data, blogs/forums of social groups, physician reports, and/or studies, among other information.

More specific embodiments of the present disclosure are directed to a scale that provides various features including communicating user-sensitive data other user devices, such as a smartwatch, smartphone, smartbed and/or smartcup, to aggregate and communicate user-sensitive data. The scale, such as a body weight scale, provides various features such as collecting scale-obtained data from a user while the user is standing on the platform apparatus, aggregating user-sensitive data from a plurality of other user devices with the scale-obtained data, and outputting the aggregated user-sensitive data to external circuitry using a secure connection to a server. In various aspects, the aggregated user-sensitive data is output in response to verifying a scale-based biometric from the user. In a number of specific embodiments, the platform apparatus includes hardware security circuitry, such as a hardware token that provides a hardware key to provide additional security. The user-sensitive data is provided to the scale from the user devices in response to secure access to the scale via a scale-based biometric and is output to the external circuitry, such as a standalone CPU and/or a server CPU, in response to the scale-based biometric. In various aspects, the levels of verification of the user and/or encryption of the data is based on the sensitivity of the data sent and/or the circuitry the data is sent to. For example, the levels of verification include different levels of scale-base biometrics, dual authorization of the scale and the external circuitry, hardware key, software key, and/or types of coding. The platform apparatus, in various aspects, is not accessed by external sources and outputs data to external circuitry and is thereby a secure-source to use as a hub for aggregating user-sensitive data and outputting the same based on authorization of the user.

Security measures performed by the scale, in a number of embodiments, are dependent on the user-sensitivity of the data. For example, the scale has a hierarchy of user identity verifications to authorize the communications and/or security measures used on the user data depending on the sensitivity of the user data being output. In various embodiments, the hierarchy includes a plurality of different scale-based biometrics, data encryption, hardware token key, software token key, among other security measures. For example, the different levels of user data are authorized for communication based on different levels of biometrics. In various specific embodiments, the scale outputs a first (lower-sensitivity) set of user data to the external circuitry in response to identifying a lower level biometric and outputs a second (greater and/or higher-sensitivity) set of user data in response to identifying a higher level biometric, such that higher security data is only communicated when a higher level biometric is verified.

The scale can acts as a hub to collect data from a variety of sources. The sources include various user devices, such as a smartwatch, fitness tracking device, smartphone, smartbed, among other devices, medical devices (implanted or otherwise), and other external circuitry, as further described herein. The scale can incorporate a web server (URL) that allows secure, remote access to the collected data. For example, the secure access can be used to provide further analysis and/or services to the user.

As used herein, a user device includes processing circuitry and output circuitry to collect various data (e.g., signals) and communicate the data to the scale and/or other circuitry. Example user devices include cellphones, tablets, standalone servers, among other devices. A wearable device is a user device (and/or a remote user-physiologic device) that is worn by a user, such as on a user's wrist, head, or chest. Example wearable devices include smartwatches and fitness bands, smartglasses, chest heart monitors, etc. A remote user-physiologic device is a user device (and/or a wearable device) that further includes sensor circuitry or other circuit to collect physiologic data from the user, and, can optionally be in secured communication with the scale or other circuitry. Example remote user-physiological devices include smartwatches or fitness bands that collect heart rate and/or ECG and/or body temperature, medical devices, implanted medical devices, smartbeds, among other devices. Example physiologic data collected by remote user-physiologic devices includes glucose measurements, blood pressure, ECG or other cardio-related data, body temperature, among other data. As used herein, the terms “user device”, “wearable device”, and “remote user-physiologic device” can be interchangeably used, as one of skill may appreciate that in specific examples, a particular device may be considered one or more of a user device, a wearable device, a remote user-physiologic device. As a specific example, a particular remote user-physiologic device is a smartwatch and can be referred to as a wearable device or a user device. In other aspects, the remote user physiologic device may not be a wearable device, such as a medical device that is periodically or temporarily used.

In accordance with a number of embodiments, physiological parameter data is collected using an apparatus, such as a weighing scale or other platform device that the user stands on. The user (e.g., owners of a scale or persons related to the owner, such as co-workers, friends, roommates, colleagues), may use the apparatus in the home, office, doctors office, or other such venue on a regular and frequent basis. The present disclosure is directed to a substantially-enclosed apparatus, as would be a weighing scale, wherein the apparatus includes a platform which is part of a housing or enclosure and a user-display to output user-specific information for the user while the user is standing on the platform. The platform includes a surface area with electrodes that are integrated and configured and arranged for engaging a user as he or she steps onto the platform. Within the housing is processing circuitry that includes a CPU (e.g., one or more computer processor circuits) and a memory circuit with user-corresponding data stored in the memory circuit. The platform, over which the electrodes are integrated, is integrated and communicatively connected with the processing circuitry. The processing circuitry is programmed with modules as a set of integrated circuitry which is configured and arranged for automatically obtaining a plurality of measurement signals (e.g., signals indicative of cardio-physiological data) from the plurality of electrodes. The processing circuitry generates, from the signals, cardio-related physiologic data manifested as user-data.

The scale, in various embodiments, includes input/output circuitry that receives data from other user devices and outputs various data to other external circuitry, such as a standalone user CPU and/or a server CPU (e.g., at a datacenter). For example, using the input/output circuitry, the scale receives various user data from one or more user devices, such as a smartphone, a smartwatch, a tablet, and/or other circuitry and devices. One or more of the user devices also include sensor circuitry and collects signals from the user indicative of the user's identity and cardio-physiological measurements, but at a different biological point of the user than the scale. For example, a smartphone or smartwatch is located near the user's hand and the scale is located near the user's feet. Further, the other user devices, in some embodiments, are used more often than the scale and/or used to collect data that the scale cannot, such as exercise logging and sleep habits. Thereby, data obtained by the scale and the user device is aggregated and/or combined and used to determine various cardio-related data that is of a higher quality (e.g., more accurate, less noise, more information) and/or more detail than data from one of the respective devices.

The aggregated data from the scale and the one or more user devices, in various embodiments, is compared to trigger data to determine if the user is at risk for a condition. The trigger data is stored directly on a memory circuit of the scale and/or is stored on a memory circuit of the standalone user CPU (and accessible by the scale). The trigger data includes values of various user data that indicate that the user is at risk for a condition (e.g., has a likelihood above a particular threshold of having or being at risk for the condition). In response to a match with the trigger data, the scale indicates a potential risk to the user and prompts the user to indicate if they would like more information. In response to the user indicating they would like further information, the enterprise system filters the user data for data correlated with the condition and filters the Internet for various data regarding the condition and/or matching the filtered user data. Thereby, the scale-based enterprise system is used as a medical analytic driver that filters the Internet based on user data related to a condition and trigger data.

In response to the filter, the user and/or the scale, in various embodiments, are used to further assess the condition of the user and/or obtain additional information. For example, the user views various data on the R-GUI, such as generic health information about the condition, articles about the condition, and/or blogs and/or forums of social groupings that are identified using the filter. The scale is used to further assess the condition of the user by performing additional tests (e.g., body-mass-index, QRS complex over time) and/or asking the user questions. In various embodiments, the user data is provided to a physician to confirm the diagnosis. In response to confirmation of the diagnosis, the scale is modified with the confirmed diagnosis. Optionally, the scale is modified to aggregate data from additional devices, such as an ECG tracking device, and/or to obtain additional parameters, such as prescription drug titration, weight loss monitoring and/or goals, exercise goals, and/or stress test.

In accordance with various embodiments, the user data is based on sensing, detection, and quantification of at least two simultaneously acquired impedance-based signals. The simultaneously acquired impedance-based signals are associated with quasi-periodic electro-mechanical cardiovascular functions, and simultaneous cardiovascular signals measured by the impedance sensors, due to the beating of an individual's heart, where the measured signals are used to determine at least one cardiovascular related characteristic of the user for determining the heart activity, health, or abnormality associated with the user's cardiovascular system. The sensors can be embedded in a user platform, such as a weighing scale-based platform, where the user stands stationary on the platform, with the user's feet in contact with the platform, where the impedance measurements are obtained where the user is standing with bare feet.

In certain embodiments, the plurality of impedance-measurement signals includes at least two impedance-measurement signals between the one foot and the other location. Further, in certain embodiments, a signal is obtained, based on the timing reference, which is indicative of synchronous information and that corresponds to information in a BCG. Additionally, the methods can include conveying modulated current between selected ones of the electrodes. The plurality of impedance-measurement signals may, for example, be carried out in response to current conveyed between selected ones of the electrodes. Additionally, the methods, consistent with various aspects of the present disclosure, include a step of providing an IPG measurement within the one foot. Additionally, in certain embodiments, the two electrodes contacting one foot of the user are configured in an inter-digitated pattern of positions over a base unit that contains circuitry communicatively coupled to the inter-digitated pattern. The circuitry uses the inter-digitated pattern of positions for the step of determining a plurality of pulse characteristic signals based on the plurality of impedance-measurement signals, and for providing an IPG measurement within the one foot. As discussed further herein, and further described in U.S. patent application Ser. No. 14/338,266 filed on Oct. 7, 2015, which is herein fully incorporated by reference for its specific teaching of inter-digitated pattern and general teaching of sensor circuitry, the circuitry can obtain the physiological data in a number of manners.

In medical (and security) applications, for example, the impedance measurements obtained from the plurality of integrated electrodes can then be used to provide various cardio-related information that is user-specific including, as non-limiting examples, synchronous information obtained from the user and that corresponds to information in a ballistocardiogram (BCG) and impedance plethysmography (IPG) measurements. By ensuring that the user, for whom such data was obtained, matches other bio-metric data as obtained concurrently for the same user, medical (and security) personnel can then assess, diagnose and/or identify with high degrees of confidence and accuracy.

In a number of a specific embodiments, the user stands on the scale. The scale, responsive to the user, transitions from a low-power mode of operation to a higher-power mode of operation. The scale may attempt to establish communication with another user device. However, the communication is not activated until authorization data is obtained by the scale from the user device and/or until a scale-based biometric is identified. For example, the scale collects signals using the data-procurement circuitry. From the collected signals, the scale identifies a scale-based biometric corresponding with the user and validates the various user data generated as corresponding to the specific user and associated with a user profile. The user device, at the same time, before or after, collects signals and/or other user data from the user. For example, while the user is standing on the platform, the user turns their cellphone from a sleep mode to on, and in the process provides a password or a biometric, such as a finger print to the cellphone. In some specific embodiments, in response to the scale receiving both the scale-based biometric and the authorization data from the user device, the scale activates communication and collects user data from the user device. In some embodiments, the signals collected by the scale and by the user device are indicative of cardio-physiological measurements.

Biometrics, as used herein, are metrics related to human characteristics and used as a form of identification and access control. Scale-based biometrics includes biometrics that are obtained using signals collected by the data-procurement circuitry of the scale (e.g., using electrodes and/or force sensors). Example scale-based biometrics include foot length, foot width, weight, voice recognition, facial recognition, an ECG-to-BCG timing relationship, BCG or ECG characteristics, a passcode tapped and/or picture drawn with a foot of the user on a foot-controlled user interface (FUI)/GUI of the user display, among other biometrics. In some specific embodiments, a scale-based biometric includes a toe-print (e.g., similar to a finger print) that is recognized using a toe-print reader on the FUI/GUI of the scale. The toe print can be used as a secure identification of the user. In other embodiments, the scale-based biometric includes a finger print captured using a user device in communication with the scale (e.g., a cellphone or tablet having finger print recognition technology). In some specific embodiments, a wearable device, such as a ring, wristband, and/or ankle bracelet can be used to positively identify a user, with or without biometrics.

In various embodiments, the user device collects signals using electrodes that are integrated with and/or within the user device, such as electrodes added as a cover to the cellphone and that are in communication with the cellphone. The user device, using the collected signals, generates cardio-physiologic measurements. The data obtained by the scale and the user device is aggregated and/or combined to provide additional information to the user and/or to track progress of the user, among other features.

The scale aggregates the data from the user devices and the scale and compares the aggregated data to trigger data to identify if the user is at risk for a condition. In response to a match to the trigger data, the scale prompts the user to determine if the user would like additional information. In response to the user indicating they would like additional information, the user data from the scale and the user devices are filtered for data correlating with the condition. Further, the internet is filtered (e.g., searched) for data correlating with the condition and the filtered user data. The user, using the R-GUI and/or a user interface of the scale, is provided access to various generic health information, articles, blogs/forums or social groupings, and other data based on the filter of the Internet using the data that correlates with the condition and the trigger data. Further, the scale is used to perform additional test and/or asks questions based on the filter of the Internet. For example, the filter of the Internet identifies new symptoms that were not part of the trigger data and/or additional parameters to track. Further, a physician, in some embodiments, is provided access to the aggregated user data for confirmation of a diagnosis. In response to a confirmed diagnosis, the scale is modified to include diagnosis data and/or to add additional device and/or parameters to track. In various embodiments, the user data is compared against historical user data for the same user and used to analyze if the user's condition/treatment and risk is getting better or worse over time.

In various embodiments, user devices provide authorization data to the scale to authorize communication between the devices (e.g., for the scale to act as a hub for collecting data). Example authorization data includes data selected from the group consisting of a password, a passcode, a biometric, a cellphone ID, and a combination thereof. A user device-based biometric, in various embodiments, includes biometrics selected from the group consisting of: a finger print, voice recognition, facial recognition, DNA, iris recognition, typing rhythm, and a combination thereof, in various embodiments. Responsive to collecting the authorization data and/or verifying the authorization data as corresponding to the user, the user device outputs the authorization data to the scale. The authorization data is collected, in various embodiments, prior to, during, and/or after, the scale collects various signals. The scale can authorize communication of user data between the scale and the user device in response to verifying that the user data corresponds to the same user as is standing on the scale (e.g., based on a scale-based biometric and data in storage). In some specific embodiments, a wearable device, such as a ring, wristband, and/or ankle bracelet can be used to positively identify a user, with or without biometrics.

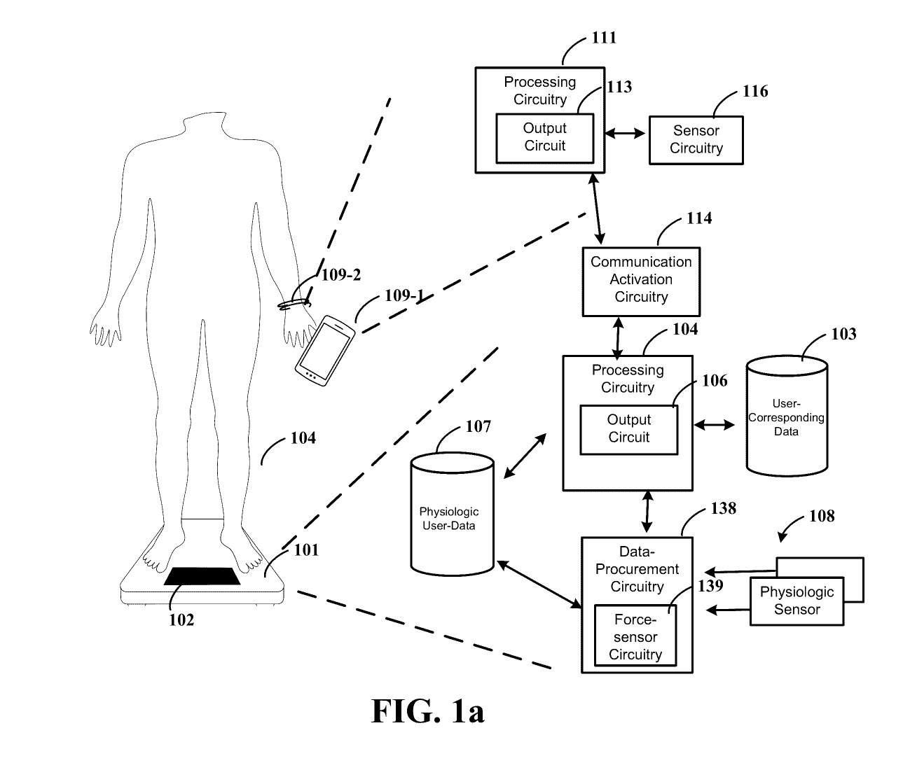

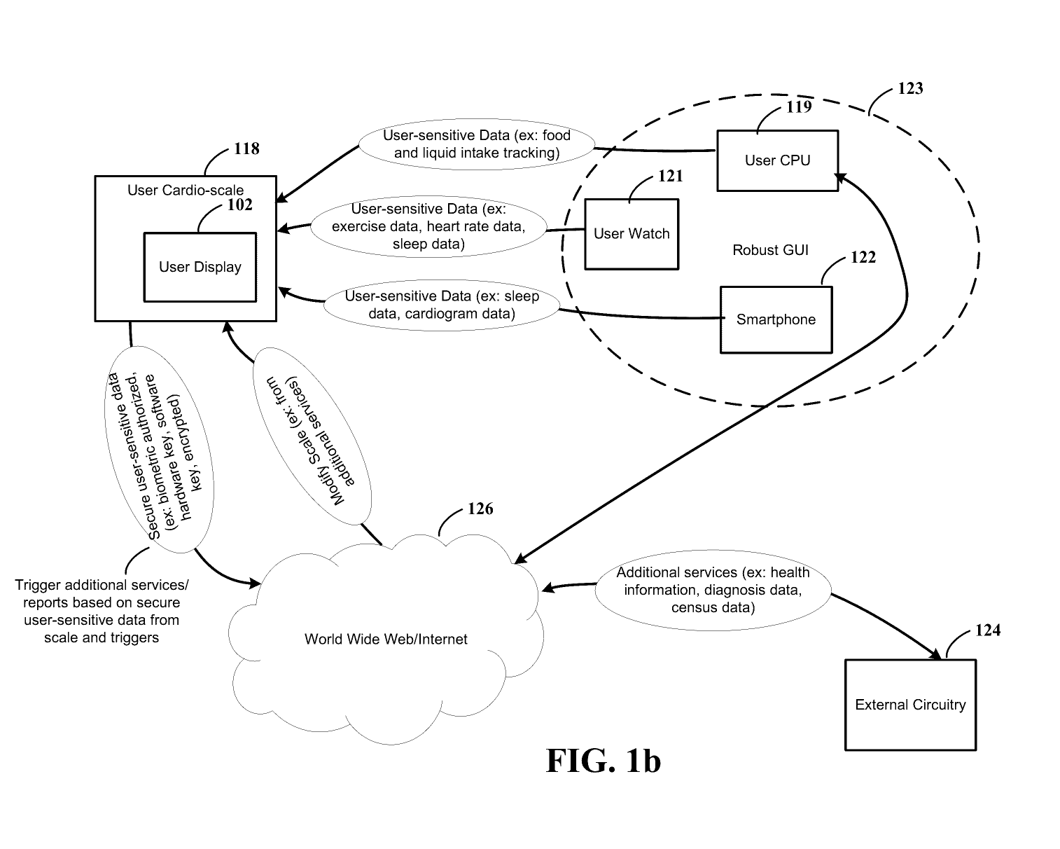

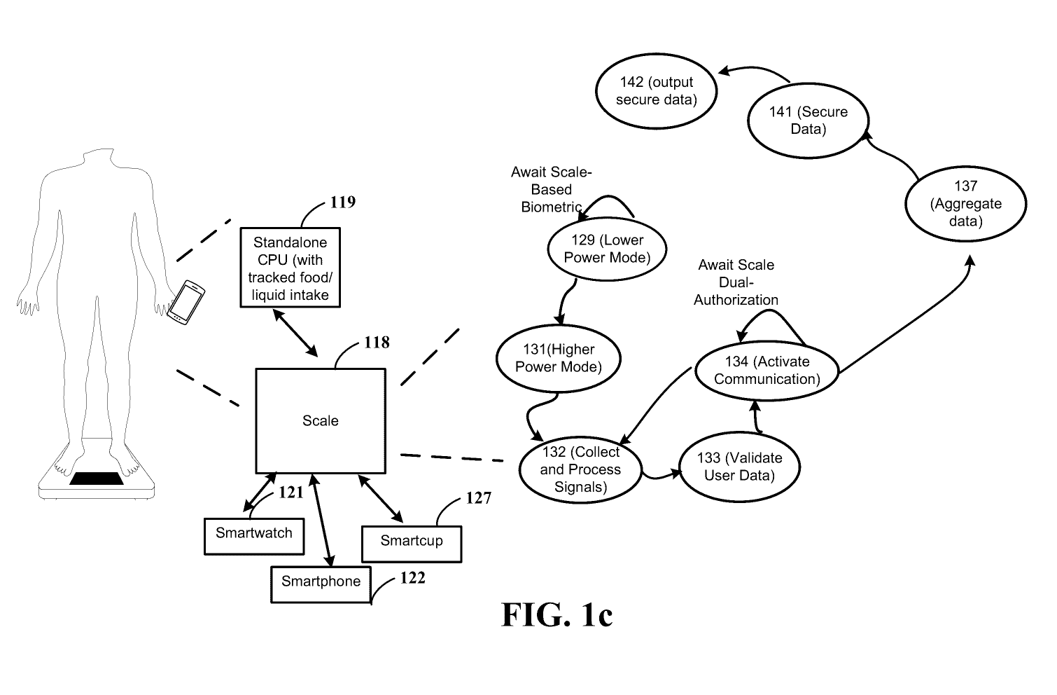

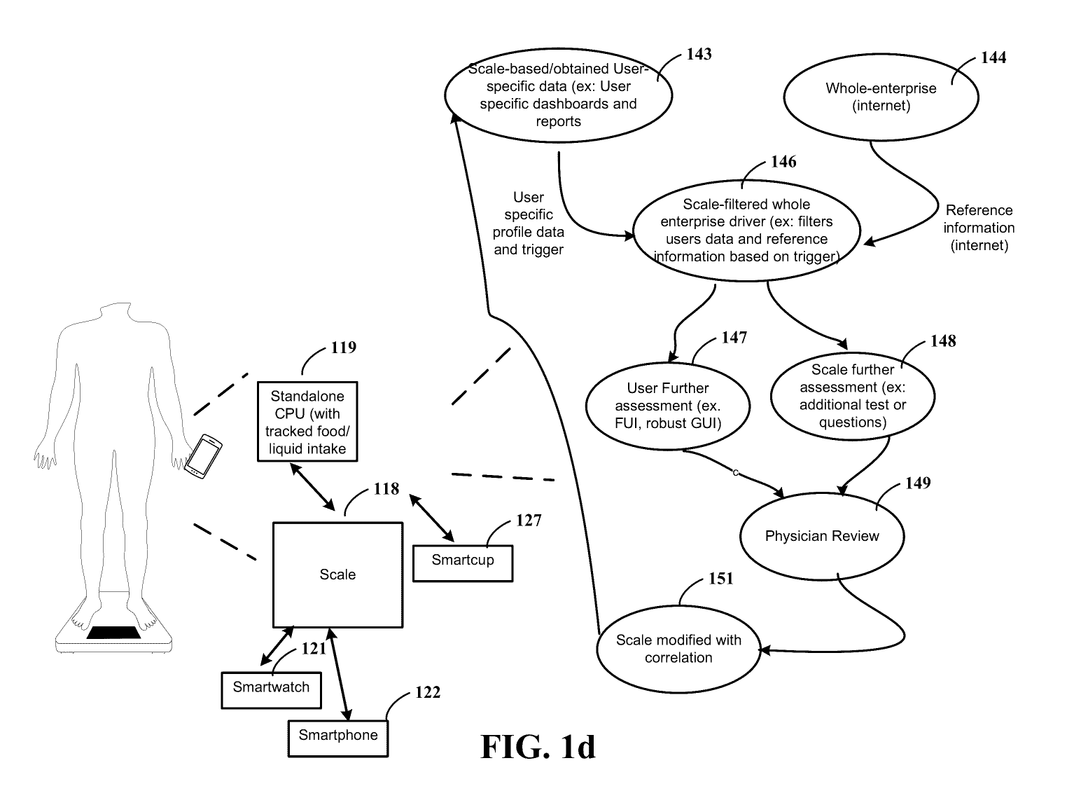

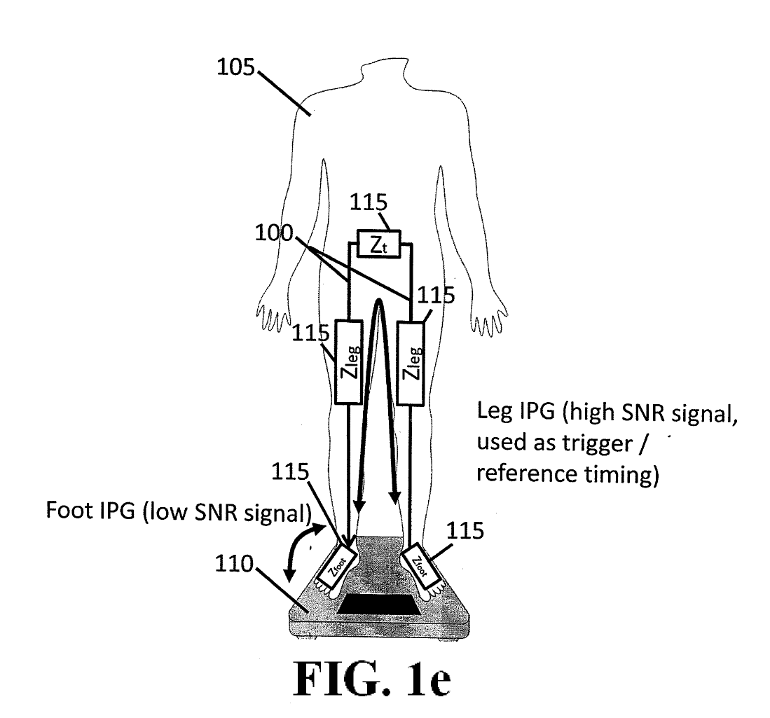

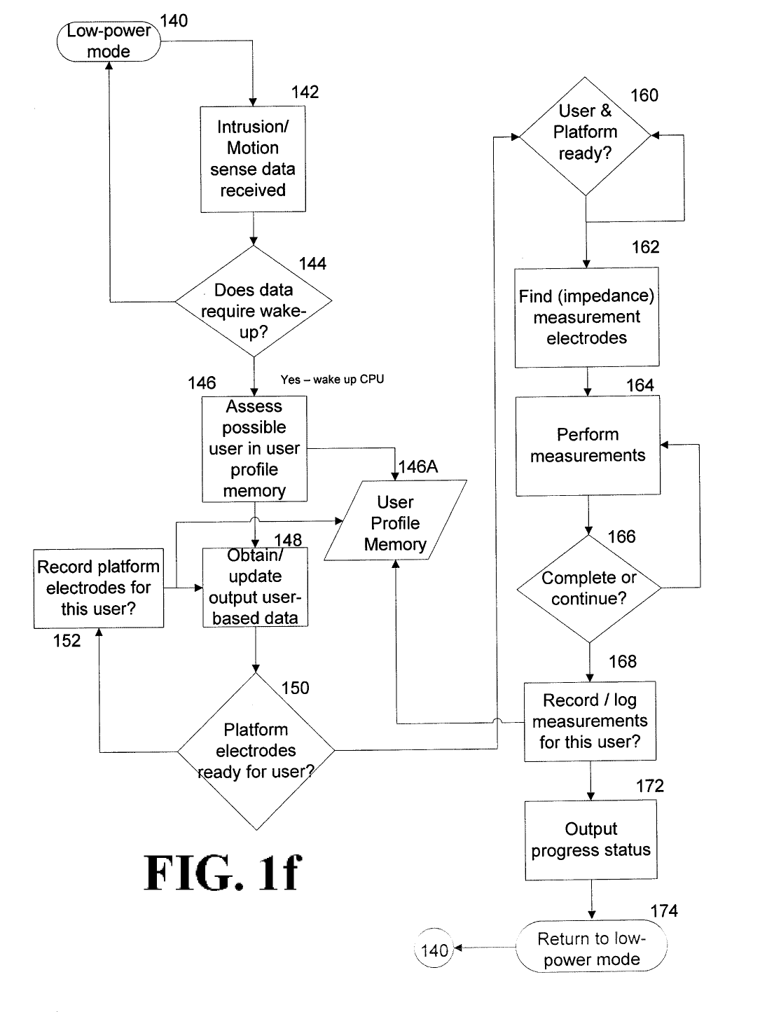

Turning now to the figures, FIG. 1a shows an apparatus consistent with aspects of the present disclosure. The apparatus includes a scale and one or more user devices (e.g., device 109-1 and/or 109-2). The scale and user devices, in various embodiments, communicate various cardio-related data and other user data. The scale collects and aggregates user data from the scale and the user devices. As may be appreciated, user data is used interchangeably with user-sensitive data herein. The scale is used to securely communicate the aggregated user data to external circuitry, such as a standalone CPU and/or server CPU. For example, the scale verifies identification and authorization of the communication using a scale-obtained biometric. In various specific embodiments, the scale adds various security measures to aggregated user data by encrypting, coding, adding a hardware and/or software key, and combinations thereof. Using the scale as a hub to aggregate and communicate user data increases security of communicating the user data as the scale, in various embodiments, is not accessed by external circuitry and/or applications. Further, the verification of the identity of the user prevents and/or avoids unintended disclosure of the user data as compared to a single authorization.

User data, interchangeably referred to as “user-sensitive data” herein, as used herein, includes data obtained by the scale and/or the user device that is related to user health, lifestyle, and/or identification. In various embodiments, both the scale and the user devices collect various user data. For example, both the scale and the user device collect cardio-related data. Alternatively, the user device collects exercise data and/or sleep data, among other data. Combining the user data from the scale and the user devices is beneficial in identifying various risks of the user for conditions, in tracking the user's progress, and/or in making suggestions to the user. However, separately sending the data to a standalone CPU and/or server CPU is time consuming and frustrating for many users. Further, the scale, in various embodiments, verifies identification of the user using a scale-based biometric to increase security of the data communication. For example, as discussed further herein, in various embodiments, the scale has a hierarchy of security measures depending on the user data. For example, different scale-obtained biometrics are used to authorize communication of different levels of user data. Further, the user can adjust the settings of the various biometrics and levels sensitivity of user data.

The scale, in various embodiments, includes a platform 101 and a user display 102. The user, as illustrated by FIG. 1a is standing on the platform 101 of the apparatus. The user display 102 is arranged with the platform 101. As illustrated by the dashed-lines of FIG. 1a, the apparatus further includes processing circuitry 104, data-procurement circuitry 138, physiologic sensors 108, communication activation circuitry 114, and an output circuit 106. That is, the dashed-lines illustrate a closer view of components of the apparatus.

The physiologic sensors 108, in various embodiments, include a plurality of electrodes and force-sensor circuitry 138 integrated with the platform 101. The electrodes and corresponding force-sensor circuitry 139 are configured to engage the user with electrical signals and to collect signals indicative of the user's identity and cardio-physiological measurements while the user is standing on the platform 101. For example, the signals are indicative of physiological parameters of the user and/or are indicative of or include physiologic data, such as data indicative of a BCG or ECG and/or actual body weight or heart rate data, among other data. Although the embodiment of FIG. 1a illustrates the force sensor circuitry 139 as separate from the physiological sensors 108, one of skill in the art may appreciate that the force sensor circuitry 139 are physiological sensors. Optionally, the user display 102 is arranged with the platform 101 and the electrodes to output user-specific information for the user while the user is standing on the platform 101. The processing circuitry 104 includes CPU and a memory circuit with user-corresponding data 103 stored in the memory circuit. The processing circuitry 104 is arranged under the platform 101 upon which the user stands, and is electrically integrated with the force-sensor circuitry 139 and the plurality of electrodes (e.g., the physiologic sensors 108).

The data indicative of the identity of the user includes, in various embodiments, user-corresponding data, biometric data obtained using the electrodes and/or force sensor circuitry, voice recognition data, images of the user, input from a user's device, and/or a combination thereof and as discussed in further detail herein. The user-corresponding data includes information about the user (that is or is not obtained using the physiologic sensors 108,) such as demographic information or historical information. Example user-corresponding data includes height, gender, age, ethnicity, exercise habits, eating habits, cholesterol levels, previous health conditions or treatments, family medical history, and/or a historical record of variations in one or more of the listed data. The user-corresponding data is obtained directly from the user (e.g., the user inputs to the scale) and/or from another circuit (e.g., a smart device, such as a cellular telephone, smart watch and/or fitness device, cloud system, etc.). The user-corresponding data 103 is input and/or received prior to and/or in response to the user standing on the scale.

In various embodiments, the processing circuitry 104 is electrically integrated with the force-sensor circuitry 139 and the plurality of electrodes and configured to process data obtained by the data-procurement circuitry 138 while the user is standing on the platform 101. The processing circuitry 104, for example, generates cardio-related physiologic data 107 corresponding to the collected signals and that is manifested as user data. Further, the processing circuitry 104 generates data indicative of the identity of the user, such as a scale-based biometric, a user ID and/or other user identification metadata. The user ID is identified, for example, in response to confirming the identification of the user using the collected signals indicative of the user's identity (e.g., a scale-based biometric).

For example, the scale identifies the user, in various embodiments, by verifying a scale-based biometric using the signals indicative of the user's identity and a user profile corresponding to the user. The scale can identify the user based on the time of day, length of foot, shape of foot, toe print, toe-tapped password, spoken words from the user, weight, height, facial features, and among other biometrics or identification data. A plurality of users may use the scale and configure the scale to include user profiles corresponding to each respective user. The user profiles include various scale-obtained biometrics that are learned by the scale (such as in an initialization mode) and used to identify the user. For example, the scale compares collected signals to the user profile to verify the scale-based biometric. In response to a match with one of the user profiles, the scale identifies the user standing on the scale as the user corresponding to the matching user profile.

The user data collected by the scale, in some embodiments, includes the raw signals, body weight, body mass index, heart rate, body-fat percentage, cardiovascular age, balance, tremors, among other non-regulated physiologic data. The user data collected by the scale can further includes force signals, PWV, weight, heartrate, BCG, balance, tremors, respiration, data indicative of one or more of the proceeding data, and/or a combination thereof. In some embodiments, the user data includes the raw force signals and additional physiologic parameter data is determined using external circuitry. Alternatively, the user data can include physiologic parameters such as the PWV, BCG, IPG, ECG that are determined using signals from the data-procurement circuitry and the external circuitry (or the processing circuitry 104 of the scale) can determine additional physiologic parameters (such as determining the PWV using the BCG) and/or assess the user for a condition or treatment using the physiologic parameter. In various embodiments, the processing circuitry 104, with the user display 102, displays at least a portion of the user data to the user. For example, user data that is not regulated is displayed to the user, such as user weight. Alternatively and/or in addition, the user data is stored. For example, the user data is stored on the memory circuit of the processing circuitry (e.g., such as the physiological user data database 107 illustrated by FIG. 1a). The processing circuitry 104, in various embodiments, correlates the collected user data (e.g., physiologic user data) with user-corresponding data, such as storing identification metadata that identifies the user with the respective data.

An algorithm to determine the physiologic data from raw signals can be located on the scale, on another device (e.g., external circuitry, cellphone), and on a Cloud system. For example, the Cloud system can learn to optimize the determination and program the scale to subsequently perform the determination locally. The Cloud system can perform the optimization and programming for each user of the scale.

The scale can optionally collect physiologic data from other devices, such as medical devices (implantable or not), user devices, wearable devices, and/or remote-physiological devices. The data can include glucose measurements, blood pressure, ECG or other cardio-related data, body temperature, among other physiologic data. In various embodiments, the user devices can include implantable medical devices and/or other medical devices, such as a pacemaker that securely shares data to the scale. Further, the scale can act as a hub to collect data from a variety of sources. The sources includes the above-noted user devices. The scale can incorporate a web server (URL) that allows secure, remote access to the collected data. For example, the secure access can be used to provide further analysis and/or services to the user. The scale and other device (or external circuitry) can pair and/or otherwise communication in response to a verification or authorization of the communication, which can be based on confirming identification of the other device, and that the same user is using the other device and the scale, and/or a scale-based biometric that is recognized, as further described herein.

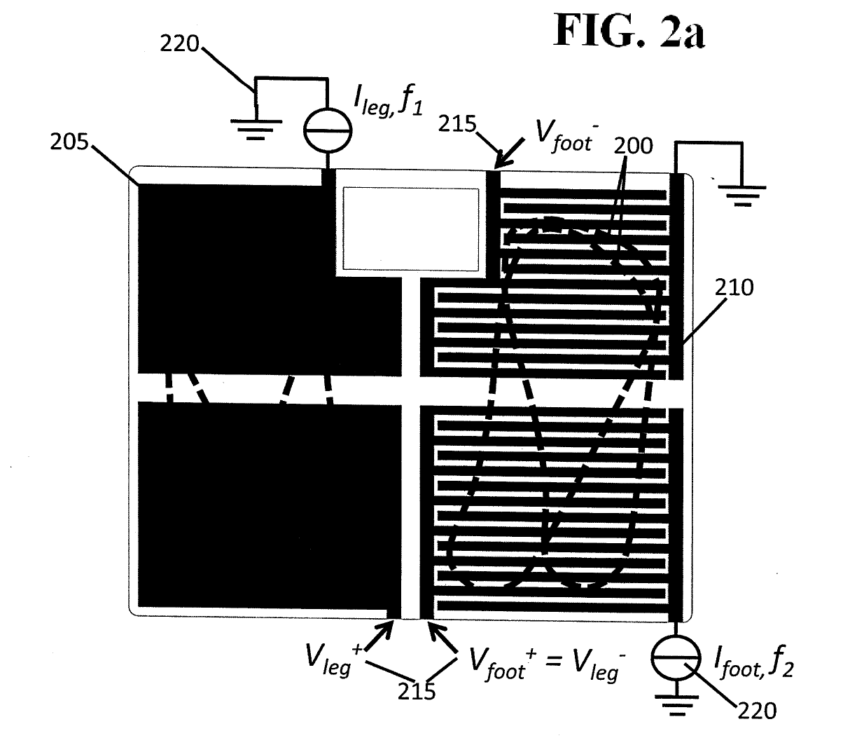

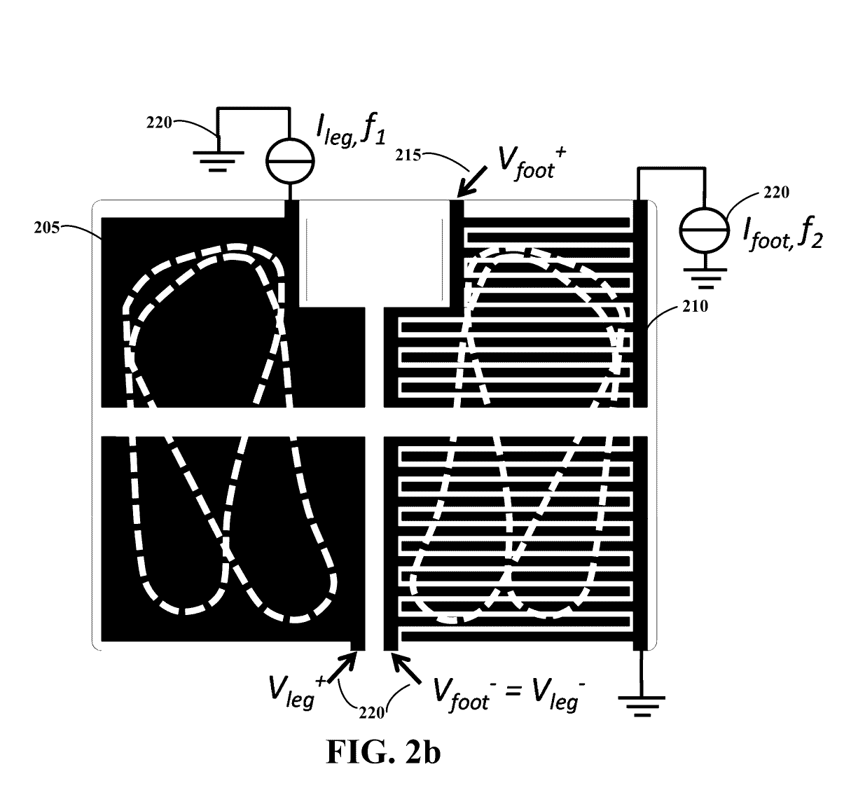

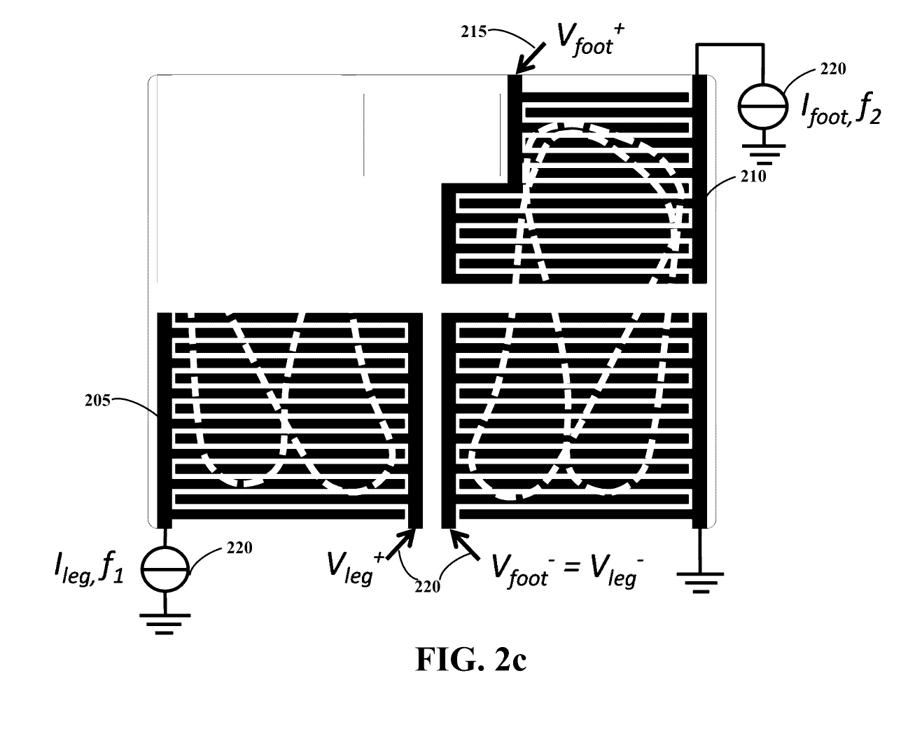

In various embodiments, in response to the user standing on the platform 101, the processing circuitry 104 transitions the scale from a reduced power-consumption mode of operation to at least one higher power-consumption mode of operation. As discussed further herein with regard to FIG. 2a, the different modes of operation, in some embodiments, include a sleep mode that uses a reduced amount of power and an awake mode that uses an additional amount of power as compared to the sleep mode. In a number of embodiments, the user display 102, data-procurement circuitry 138, and the processing circuitry 104 (among other components) transition from the reduced power-consumption mode of operation to the higher power-consumption mode of operation.

The processing circuitry 104 identifies a scale-based biometric of the user using the collected signals. For example, the scale-based biometric includes foot length, foot width, weight, voice recognition, facial recognition, and a combination thereof. In various embodiments, the scale-based biometric corresponds to a user ID and is used to verify the identity of the user. Using the scale-based biometric, the user data is validated as concerning the user associated with the scale-based biometric. The user data includes data indicative of the user's identity and the generated cardio-related physiologic data.

The one or more user devices, e.g., device 109-1 and/or 109-2, as illustrated, are not integrated within the scale and, in various embodiments, includes a cellphone, a smartwatch, other smart devices, a tablet, a (photo) plethysmogram a two terminal ECG sensor, and a combination thereof. Each user device includes processing circuitry 111 and an output circuit 113. Optionally, one or more user device includes sensor circuitry 116. The user devices are configured to collect various signals. For example, the user device collects signals indicative of the user's identity. The collected signals indicative of the user's identity include the authorization data to authorize use of the user device and, optionally, is sent to the scale to authorize communication. For example, the user device identifies the authorization data of the user using the collected signals indicative of the user's identity and, therefrom, validates the collected signals as concerning the user associated with the authorization data and/or a user profile.

Example authorization data includes data selected from the group consisting of a password, a passcode, a biometric, a cellphone ID, and a combination thereof. A remote user-physiologic device-based biometric, in various embodiments, includes biometrics selected from the group consisting of: a finger print, voice recognition, facial recognition, DNA, iris recognition, typing rhythm, and a combination thereof, in various embodiments. Responsive to collecting the authorization data and/or verifying the authorization data as corresponding to the user, the user device outputs the authorization data to the scale. The authorization data is collected, in various embodiments, prior to, during, and/or after, the scale collects various signals.

In some embodiments, the scale, optionally, receives the authorization data and, in response to both the authorization data and the scale-based biometric corresponding to the user, activates communication between the scale and the remote user-physiologic device. For example, the scale includes a communication activation circuitry 114 to activate the communication. The communication activation circuitry 114, in some embodiments, includes an AND gate to activate the communication in response to receiving both the identified scale-based biometric and the authorization data that correspond to the same user. Although embodiments are not so limited and the communication activation circuitry can include various circuit components and/or processing circuitry to activate the communication and/or verify both the scale-based biometric and the authorization data correspond to the specific user. Further, in various specific embodiments, the communication is activated in response to the scale-based biometric identified using the scale and verification of the user device based on data in a user profile corresponding with the user (e.g., identification of the user device) and/or within the user data sent by the user device.

In response to the activation, the user device outputs user data to the scale. For example, the output circuit 106 receives the user data from the remote devices and, in response, outputs the user data to the processing circuitry 104. The external circuitry is at a remote location from the scale and is not integrated with the scale. The communication, in various embodiments, includes a wireless communication and/or utilizes a cloud system.

In various embodiments, the output circuit 106 provides data to the user via a user interface. The user interface can be integrated with the platform 101 (e.g., internal to the scale) and/or can be integrated with external circuitry that is not located under the platform 101. In some embodiments, the user interface is a plurality of user interfaces, in which at least one user interface is integrated with the platform 101 and at least one user interface is not integrated with the platform 101.

A user interface includes or refers to interactive components of a device (e.g., the scale) and circuitry configured to allow interaction of a user with the scale (e.g., hardware input/output components, such as a screen, speaker components, keyboard, touchscreen, etc., and circuitry to process the inputs). The user interface is integrated with the platform (e.g., internal to the scale) and/or is integrated with external circuitry that is not located under the platform, in various aspects. A user display includes an output surface (e.g., screen) that shows text and/or graphical images (e.g., the FUI or GUI) as an output from a device to a user (e.g., cathode ray tube, liquid crystal display, light-emitting diode, organic light-emitting diode, gas plasma, touch screens, etc.) For example, output circuit 106 can provide data for display on the user display 102 the user's weight and the data indicative of the user's identity and/or the generated cardio-related physiologic data corresponding the collected signals.

The user interface can be or include a graphical user interface (GUI), a FUI, and/or voice input/output circuitry. The user interface can be integrated with the platform 101 (e.g., internal to the scale) and/or can be integrated with external circuitry that is not located under the platform 101. In some embodiments, the user interface is a plurality of user interfaces, in which at least one user interface is integrated with the platform 101 and at least one user interface is not integrated with the platform 101. Example user interfaces include input/output devices, such as display screens, touch screens, microphones, etc.

A FUI is a user interface that allows for the user to interact with the scale via inputs using their foot and/or via graphic icons or visual indicators near the user's foot while standing on the platform. In specific aspects, the FUI receives inputs from the user's foot (e.g., via the platform) to allow the user to interact with the scale. The user interaction includes the user moving their foot relative to the FUI, the user contacting a specific portion of the user display with their foot, etc. In a specific example, when the user stands on the platform of the scale, and the scale detects touching of the toe, the scale can reject the toe touch (or tap) as a foot signal (e.g., similar to wrist rejection for capacitive tablets with stylus). In some embodiments, the user display includes a touch screen and the user interaction includes the user selecting an icon, an item in a list, a virtual keyboard, among other selections, using a portion of their foot.

For example, the FUI can display various tests and/or functions that can be performed and the user can select one of the test or functions by contacting their toe with an icon of the respective test or function. In response to the selection, the scale performs the test or function. Alternatively and/or in addition, the scale is configured with a haptic, capacitive or flexible pressure-sensing upper surface, the (upper surface/tapping) touching from or by the user is sensed in the region of the surface and processed according to conventional X-Y grid Signal processing in the logic circuitry/CPU that is within the scale. By using one or more of the accelerometers located within the scale at its corners, such user data entry is sensed by each such accelerometer so long as the user's toe, heel or foot pressure associated with each tap provides sufficient force. In some embodiments, the user display is integrated with motion sense circuitry. The user interaction, in such embodiments, include the user moving their foot (with or without touching the user display). In various embodiments, the control of the FUI can be provided to a separate user device, such a user device that has previously been or is paired with the scale and that is detected by the scale. As a specific example, the scale provides a cellphone with control functions to control the display of the FUI in response to detecting the cellphone is within a threshold distance. In a specific example, when the user stands on the platform of the scale, and the scale detects touching of the toe, the scale can reject the toe touch (or tap) as a foot signal (e.g., similar to wrist rejection for capacitive tablets with stylus).

A GUI is a user interface that allows the user to interact with the scale through graphical icons and visual indicators. As an example, the external circuitry includes a GUI, processing circuitry, and output circuitry to communicate with the processing circuitry of the scale. The communication can include a wireless or wired communication. Example external circuitry can include a wired or wireless tablet, a cellphone (e.g., with an application), a smartwatch or fitness band, smartglasses, a laptop computer, among other devices. In other examples, the scale includes a GUI and voice input/output circuitry (as further described below) integrated in the platform 101. The user interact with the scale via graphical icons and visual indicators provided via the GUI and voice commands from the user to the scale.

Voice input/output circuitry (also sometimes referred to as speech input/output) can include a speaker, a microphone, processing circuitry, and other optional circuitry. The speaker outputs computer-generated speech (e.g., synthetic speech, instructions, messages) and/or other sounds (e.g., alerts, noise, recordings, etc.) The computer-generated speech can be predetermined, such as recorded messages, and/or can be based on a text-to-speech synthesis that generates speech from computer data. The microphone captures audio, such a voice commands from the user and produces a computer-readable signal from the audio. For example, the voice input/output circuitry can include an analog-to-digital converter (ADC) that translates the analog waves captured by the microphone (from voice sounds) to digital data. The digital data can be filtered using filter circuitry to remove unwanted noise and/or normalize the captured audio. The processing circuitry (which can include or be a component of the processing circuitry 104) translates the digital data to computer commands using various speech recognition techniques (e.g., pattern matching, pattern and feature matching, language modeling and statistical analysis, and artificial neural networks, among other techniques).

The scale receives the user data and validates the user data as concerning a specific user associated with a user profile (based on the communication activation and/or a user ID within the user data), such as using authorization data and/or other identifying data in the user data. The user data includes data collecting using sensor circuitry, such as accelerometers and/or electrodes, and/or using processing circuitry. For example, a user inputs user-sensitive data to one or more of the user devices.

In some embodiments, the user device, using the sensor circuitry 116 and the processing circuitry 111, collects signals indicative of cardio-physiological data. For example, the sensor circuitry 116, includes electrodes and/or other circuitry configured and arranged to collect the signals. The signals include recordings of electrical activity of the user's heart over a period of time and that are collected by placing electrodes on the user's body. The electrodes detect electrical changes on the skin and/or other surface that arise from the heart muscle depolarizing during each heartbeat. That is, the signals are indicative, in various embodiments, of an ECG of the user. The processing circuitry 111 of the user device receives the collected signals, and, therefrom generates the cardio-physiological data (e.g., the ECG). Thereby, the user device includes a two-terminal ECG sensor and/or a plethysmogram sensor, in various embodiments.

The scale can aggregate the user data obtained by the scale (e.g., user data) with the user data from the one or more user device. For example, the aggregation includes combining and/or correlating the data. In addition, the scale securely communicates the aggregated user-sensitive data to external circuitry using a secure connection to a server, by verifying the communication using a scale-obtained biometric, and/or by performing additional security measures on the data.

In various embodiments, the scale correlates portions of the user data obtained by the scale with the user-sensitive data. The correlation includes placing the data in phase, in the same and/or similar time range, in the same and/or similar time scale, and/or other correlation. For example, the cardio-physiologic data from the scale, in a number of embodiments, includes data indicative of a BCG and the cardio-physiologic data from the remote user-physiologic device includes data indicative of an ECG. The correlation can include correcting the data to get true phase change between the BCG and ECG. In other embodiments, the scale can collect an ECG from a different location than an ECG collected by the remote user-physiologic device. The correlation includes placing the ECG data from the scale in phase with the ECG data from the remote user-physiologic device, such that the two cardiogram waveforms correspond to one another. Alternatively and/or in addition, the BCG and ECG data includes time stamps and the correlation includes matching the data based on the time stamps. The correlated data is stored in a user profile corresponding with the user, such as a user profile stored on the scale.

The scale can be configured to collect data from a plurality of users. The scale differentiates between the different uses based on scale-based biometrics. The scale-obtained data includes health data that is sensitive to users, such that unintentional disclosure of scale-obtained data is not desired. Differentiating between the two or more users and automatically communicating (e.g., without further user input) user data responsive to scale-obtained biometrics, in various embodiments, provides a user-friendly and simple way to communicate data from a scale while avoiding and/or mitigating unintentional (and/or without user consent) communication. For example, the scale, such as during an initialization mode for each of the two or more users, collects user data to identify the scale-based biometrics and stores an indication of the scale-based biometrics in a user profile corresponding with the respective user. During subsequent measurements, the scale recognizes the particular user by comparing collected signals to the indication of the scale-based biometrics in the user profile. The scale, for example, compares the collected signals to each user profile of the two or more users and identifies a match between the collected signals and the indication of the scale-based biometrics. A match, in various embodiments, is within a range of values of the indication stored. Further, in response to verifying the scale-based biometric(s), a particular communication mode is authorized. In accordance with various embodiments, the scale uses a cardiogram of the user and/or other scale-obtained biometrics to differentiate between two or more users.

The scale communicates the aggregated user data, in various embodiments, by authorizing the communication based on the biometric identified and adding various security measures to the user data in response to the authorized communication. For example, in various embodiments, the user profiles are associated with a hierarchy of different levels of biometrics that enable different data to be communicated and/or to different sources. For example, in response to verifying a first biometric, the scale outputs the user's weight to the user's smartphone or other standalone CPU. In response to verifying a second biometric, the scale outputs additional data to external circuitry and/or that is more user-sensitive, as discussed further herein. In response to verifying the second biometric, the scale outputs the user data (such as higher-sensitivity user data) from the scale to the smartphone or standalone CPU, from the scale to the smartphone/standalone CPU for sending to a third party, and/or from the scale to the third party.

As an example, for user data, the above described biometrics are used as directed by the user for indicating and defining protocol to permit such data to be exported from the scale to other external circuitry. In more specific embodiments, the scale operates in different modes of data security including, for example: a default mode in which the user's body mass and/or weight is displayed regardless of any biometric which would associate with the specific user standing on the scale; another mode in which complicated data (or data reviewed infrequently) is only exported from the scale under specific manual commands provided to the scale under specific protocols; and another mode or modes in which the user-specific data that is collected from the scale is processed and accessed based on the type of data. Such data categories include categories of different level of importance and/or sensitivities such as the above-discussed high and low level data and other data that might be very specific to a symptom and/or degrees of likelihood for diagnoses. Optionally, the CPU in the scale is also configured to provide encryption of various levels of the user's sensitive data.

In some specific embodiments, the scale operates in different modes of data security and communication. The different modes of data security and communication are enabled in response to biometrics identified by the user and using the user interface (e.g., FUI). In some embodiments, the scale is used by multiple users and/or the scale operates in different modes of data security and communication in response to identifying the user and based on biometrics. The different modes of data security and communication include, for example: a first mode (e.g., default mode) in which the user's body mass and/or weight is displayed regardless of any biometric which would associate with the specific user standing on the scale and no data is communicated to external circuitry; a second mode in which complicated/more-sensitive data (or data reviewed infrequently) is only exported from the scale under specific manual commands provided to the scale under specific protocols and in response to a biometric; and a third mode or modes in which the user-specific data that is collected from the scale is processed and accessed based on the type of data and in response to a biometric. Such data categories include categories of different levels of importance and/or sensitivities such as the above-discussed high and low level data and other data that might be very specific to a symptom and/or degrees of likelihood for diagnoses. Optionally, the CPU in the scale is also configured to provide encryption of various levels of the user's sensitive data.

Examples of security measures in place include firewalls, encryption schemes used, access to the requesters database by external sources, authentication of people when accessing the data, such as tokens, passwords, finger print, and/or biometrics, among other security measures.

In some embodiments, the different modes of data security and communication are enabled in response to recognizing the user standing on the scale using a biometric and operating in a particular mode of data security and communication based on user preferences and/or services activated. For example, the different modes of operation include the default mode (as discussed above) in which certain data (e.g., categories of interest, categories of user data, or historical user data) is not communicated from the scale to external circuitry, a first communication mode in which data is communicated to external circuitry as identified in a user profile, a second or more communication modes in which data is communicated to a different external circuitry for further processing. The different communication modes are enabled based on biometrics identified from the user and user settings in a user profile corresponding with each user. Although the different communications are referred to as “modes”, one of skill in the art may appreciate that the communications in the different modes may not (or may) include different media and channels. The different communication modes can include different devices communicated to and/or different data that is communicated based on sensitivity of the data and/or security of the devices.

In a specific embodiment, a first user of the scale may not be identified and/or have a user profile set up. In response to the first user standing on the scale, the scale operates in a default mode. During the default mode, the scale displays the user's body mass and/or weight on the user display and does not output user data. The scale, in various embodiments, displays a prompt (e.g., an icon) on the FUI indicating the first user can establish a user profile. In response to the user selecting the prompt, the scale enters an initialization mode. During the initialization mode, the scale asks the user various questions, such as identification of external circuitry to send data to, identification information of the first user, and/or demographics of the user. The user provides inputs using the FUI to establish various communication modes associated with the user profile and scale-based biometrics to enable the one or more communication modes. The scale further collects user data to identify the scale-based biometrics and stores an indication of the scale-based biometric in the user profile such that during subsequent measurements, the scale recognizes the user and authorizes a particular communication mode. Alternatively, the user provides inputs for the initialization mode using another device that is external to the scale and in communication with the scale (e.g., a cellphone).

A second user of the scale has a user profile set up that indicates the user would like data communicated to a computing device of the user. When the second user stands on the scale, the scale recognizes the second user based on a biometric and operates in a first communication mode. During the first communication mode, the scale outputs at least a portion of the user data to an identified external circuitry. For example, the first communication mode allows the user to upload data from the scale to a user identified external circuitry (e.g., the computing device of the user). The information may include user data and/or user information that has low-user sensitivity, such as user weight and/or bmi. In the first communication mode, the scale performs the processing of the raw sensor data and/or the external circuitry. For example, the scale sends the raw sensor data and/or additional health information to a user device of the user. The computing device may not provide access to the raw sensor data to the user and/or can send the raw sensor data to another external circuitry for further processing in response to a user input. For example, the computing device can ask the user if the user would like generic health information and/or regulated health information as a service. In response to receiving an indication the user would like the generic health information and/or regulated health information, the computing device outputs the raw sensor data and/or non-regulated health information to another external circuitry for processing, providing to a physician for review, and controlling access, as discussed above.

In one or more additional communication modes, the scale outputs raw sensor data to an external circuitry for further processing. For example, during a second communication mode and a third communication, the scale sends the raw sensor data and/or other data to external circuitry for processing. Using the above-provided example, a third user of the scale has a user profile set up that indicates the third user would like scale-obtained data to be communicated to an external circuitry for further processing, such as to determine generic health information. When the third user stands on the scale, the scale recognizes the third user based on one or more biometrics and operates in a second communication mode. During the second communication mode, the scale outputs raw sensor data to the external circuitry. The external circuitry identifies one or more risks, and, optionally, derives generic health information. In some embodiments, the external circuitry outputs the generic health information to the scale. The scale, in some embodiments, displays a synopsis of the generic health information and/or outputs a full version of the generic health information to another user device for display (such as, using the filter described above) and/or an indication that generic health information can be accessed.

A fourth user of the scale has a user profile set up that indicates the fourth user has enabled a service to access regulated health information. When the fourth user stands on the scale, the scale recognizes the user based on one or more biometrics and operates in a fourth communication mode. In the fourth communication mode, the scale outputs raw sensor data to the external circuitry, and the external circuitry processes the raw sensor data and controls access to the data. For example, the external circuitry may not allow access to the regulated health information until a physician reviews the information. In some embodiments, the external circuitry outputs data to the scale, in response to physician review. For example, the output data can include the regulated health information and/or an indication that regulated health information is ready for review. The external circuitry may be accessed by the user, using the scale and/or another user device. In some embodiments, using the FUI of the scale, the scale displays the regulated health information to the user. The scale, in some embodiments, displays a synopsis of the regulated health information (e.g., clinical indication) and outputs the full version of regulated health information to another user device for display (such as, using the filter described above) and/or an indication that the regulated health information can be accessed to the scale to display. In various embodiments, if the scale is unable to identify a particular (high security) biometric that enables the fourth communication mode, the scale may operate in a different communication mode and may still recognize the user. For example, the scale may operate in a default communication mode in which the user data collected by the scale is stored in a user profile corresponding to the fourth user and on the scale. In some related embodiments, the user data is output to the external circuitry at a different time.

Although the present embodiments illustrate a number of security and communication modes, embodiments in accordance with the present disclosure can include additional or fewer modes. Furthermore, embodiments are not limited to different modes based on different users. For example, a single user may enable different communication modes in response to particular biometrics of the user identified and/or based on user settings in a user profile.

In various embodiments, the scale defines a user data table that defines types of user data and sensitivity values of each type of user data. In specific embodiments, the FUI can display the user data table. In other specific embodiments a user interface of a smartphone, tablet, and/or other computing device displays the user data table. For example, a wired or wireless tablet is used, in some embodiments, to display the user data table. The sensitivity values of each type of user data, in some embodiments, define in which communication mode(s) the data type is communicated and/or which biometric is used to enable communication of the data type. In some embodiments, a default or pre-set user data table is displayed and the user revises the user data table using the FUI. The revisions are in response to user inputs using the user's foot and/or contacting or moving relative to the FUI. Although the embodiments are not so limited, the above (and below) described control and display is provided using a wireless or wired tablet or other computing device as a user interface. The output to the wireless or wired tablet, as well as additional external circuitry, is enabled using biometrics. For example, the user is encouraged, in particular embodiments, to configure the scale with various biometrics. The biometrics include scale-based biometrics and biometrics from the tablet or other user computing device. The biometrics, in some embodiments, used to enable output of data to the tablet and/or other external circuitry includes a higher integrity biometric (e.g., higher likelihood of identifying the user accurately) than a biometric used to identify the user and stored data on the scale.

An example user data table is illustrated below:

| | User-data Type | | | | |
|---|---|---|---|---|---|
| | | Body Mass | User- | Physician- | Scale-stored |
| | Weight, | Index, user | Specific | Provided | suggestions |
| | local | specific | Advertise- | Diagnosis/ | (symptoms & |
| | weather | news | ments | Reports | diagnosis) |
| Sensi- | 1 | 3 | 5 | 10 | 9 |
| tivity | | | | | |
| (10 = | | | | | |
| highest, | | | | | |
| 1 = | | | | | |
| lowest) | | | | | |

The above-displayed table is for illustrative purposes and embodiments in accordance with the present disclosure can include additional user-data types than illustrated, such as cardiogram characteristics, clinical indications, physiological parameters, user goals, demographic information, etc. In various embodiments, the user data table includes additional rows than illustrated. The rows, in specific embodiments, include different data input sources and/or sub-data types (as discussed below). Data input sources include source of the data, such as physician-provided, input from the Internet, user-provided, from the external circuitry. The different data from the data input sources, in some embodiments, is used alone or in combination.

In accordance with various embodiments, the scale uses a cardiogram (on its own or in addition to weight, BCG, ECG, and/or various combinations) of the user and/or other scale-obtained biometrics to differentiate between two or more users. The scale-obtained data includes health data that is user-sensitive, such that unintentional disclosure of scale-obtained data is not desired. Differentiating between the two or more users and automatically communicating (e.g., without further user input) user data responsive to scale-obtained biometrics, in various embodiments, provides a user-friendly and simple way to communicate data from a scale while avoiding and/or mitigating unintentional (and/or without user consent) communication. For example, the scale, such as during an initialization mode for each of the two or more users and as previously discussed, collects user data to identify the scale-based biometrics and stores an indication of the scale-based biometrics in a user profile corresponding with the respective user. During subsequent measurements, the scale recognizes the particular user by comparing collected signals to the indication of the scale-based biometrics in the user profile. The scale, for example, compares the collected signals to each user profile of the two or more users and identifies a match between the collected signals and the indication of the scale-based biometrics. A match, in various embodiments, is within a range of values of the indication stored. Further, in response to verifying the scale-based biometric(s), a particular communication mode is authorized.

In accordance with a number of embodiments, the scale identifies one or more of the multiple users of the scale that have priority user data. The user data with a priority, as used herein, includes an importance of the user and/or the user data. In various embodiments, the importance of the user is based on parameter values identified and/or user goals, such as the user is an athlete and/or is using the scale to assist in training for an event (e.g., marathon) or is using the scale for other user goals (e.g., a weight loss program). Further, the importance of the user data is based on parameters values and/or user input data indicating a diagnosis of a condition or disease and/or a risk of the user having the condition or disease based on the scale-obtained data. For example, the scale-obtained data of a first user indicates that the user is overweight, recently had an increase in weight, and has a risk of having atrial fibrillation. The first user is identified as a user corresponding with priority user data. A second user of the scale has scale-obtained data indicating a decrease in recovery parameters (e.g., time to return to baseline parameters) and the user inputs an indication that they are training for a marathon. The second user is also identified as a user corresponding with priority user data. The scale displays indications to a user with the priority user data, in some embodiments, on how to use to the scale to communicate the user data to external circuitry for further processing, correlation, and/or other features, such as social network connections. Further, the scale, in response to the priority, displays various feedback to the user, such as user-targeted advertisements and/or suggestions. In some embodiments, only users with priority user data have data output to the external circuitry to determine risks, although embodiments in accordance with the present disclosure are not so limited.

In some embodiments, one or more users of the scale have multiple different scale-obtained biometrics used to authorize different communication modes. The different scale-obtained biometrics are used to authorize communication of different levels of sensitivity of the user data, such as the different user-data types and sensitivity values as illustrated in the above-table. For example, in some specific embodiments, the different scale-obtained biometrics include a high security biometric, a medium security biometric, and a low security biometric. Using the above illustrated table as an example, the three different biometrics are used to authorize communication of the user-data types of the different sensitivity values. For instance, the high security biometric authorizes communication of user-data types with sensitivity values of 8-10, the medium security biometric authorizes communication of user-data types with sensitivity values of 4-7, and the low security biometric authorizes communication of user-data types with sensitivity values of 1-3. The user, in some embodiments, can adjust the setting of the various biometrics and authorization of user-data types. An example high security biometric can include a ECG-to-BCG timing relationship in addition to (or on its own) one or more of a foot shape, toe tapped password and/or a toe print. A low security biometric can include a user weight, a foot size, a body-mass-index.

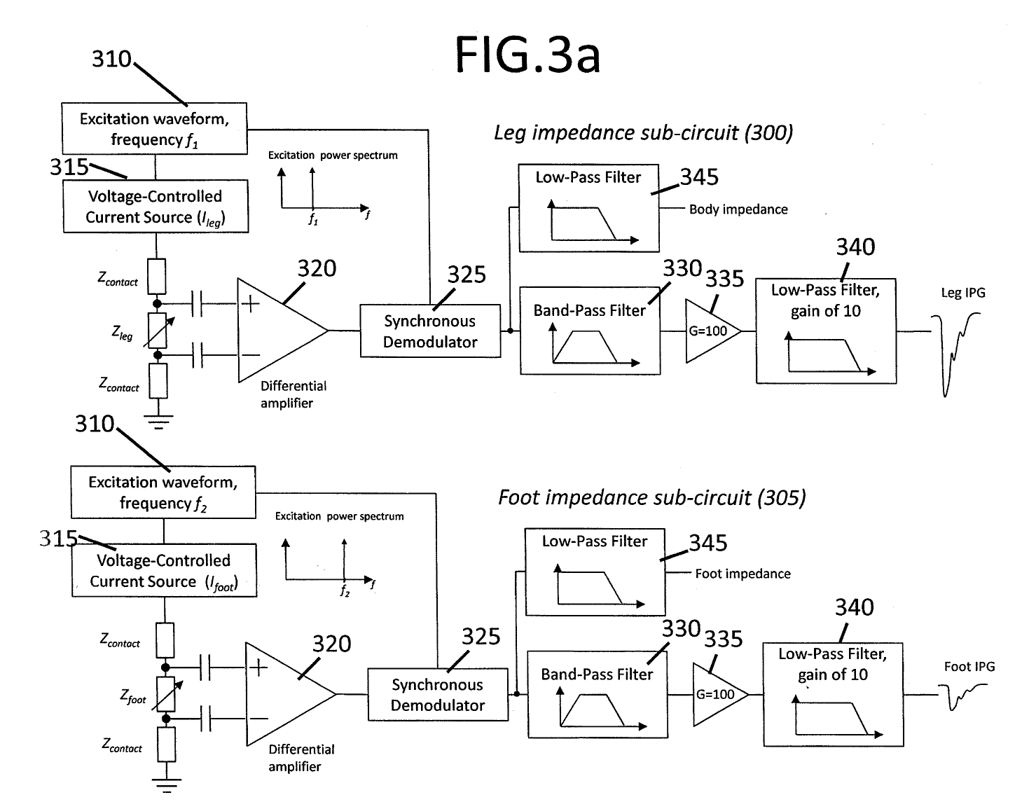

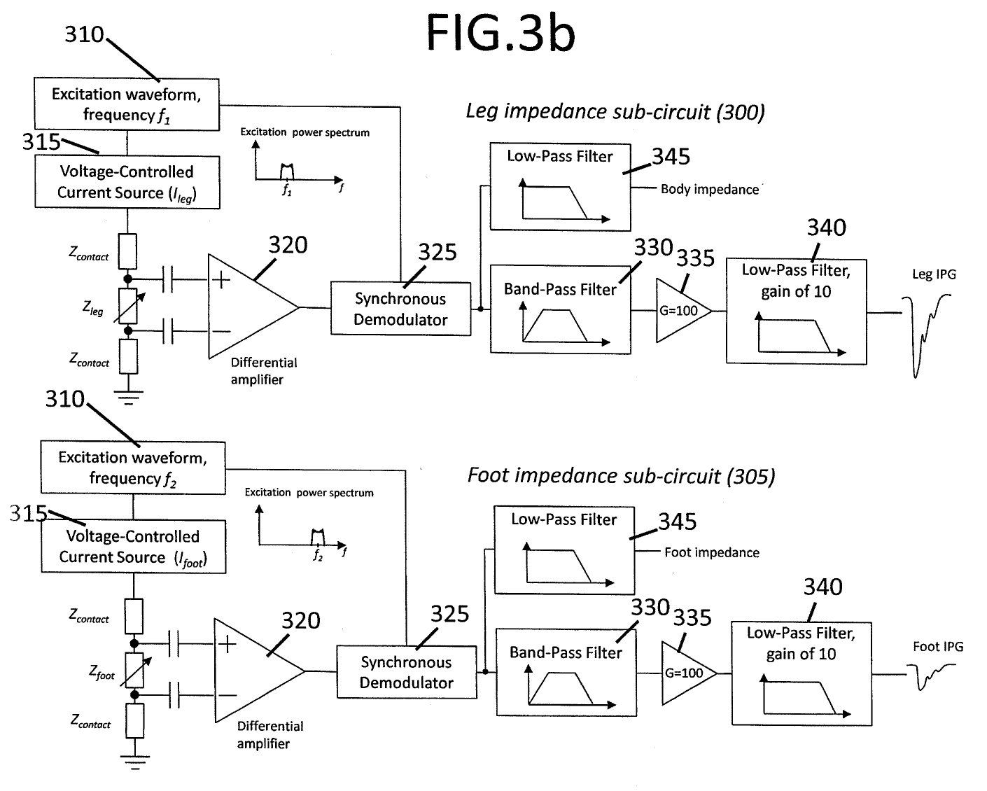

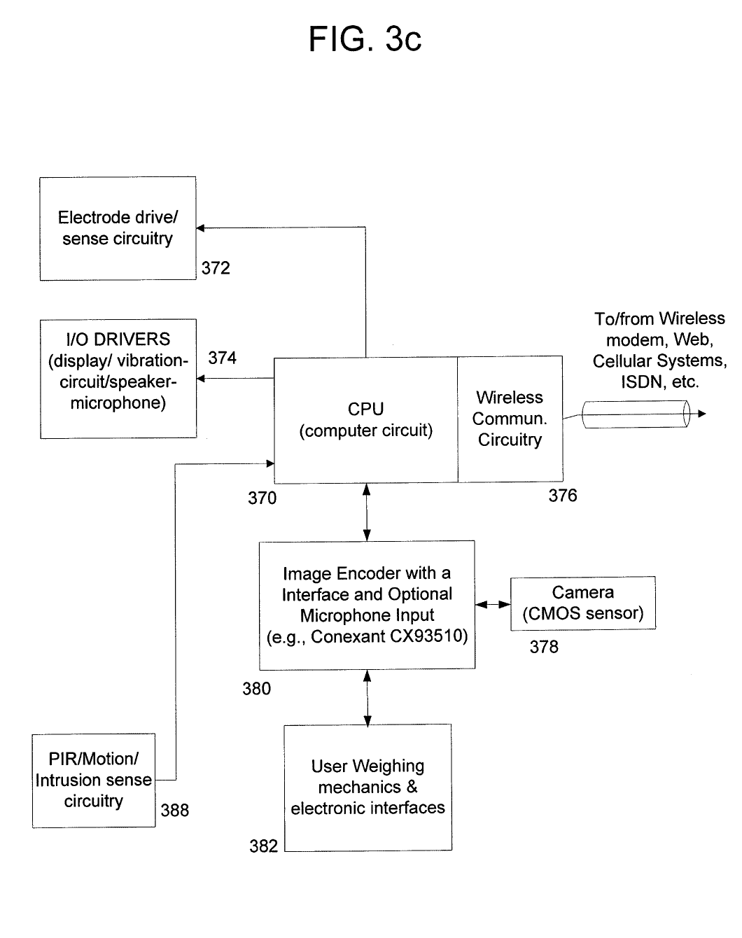

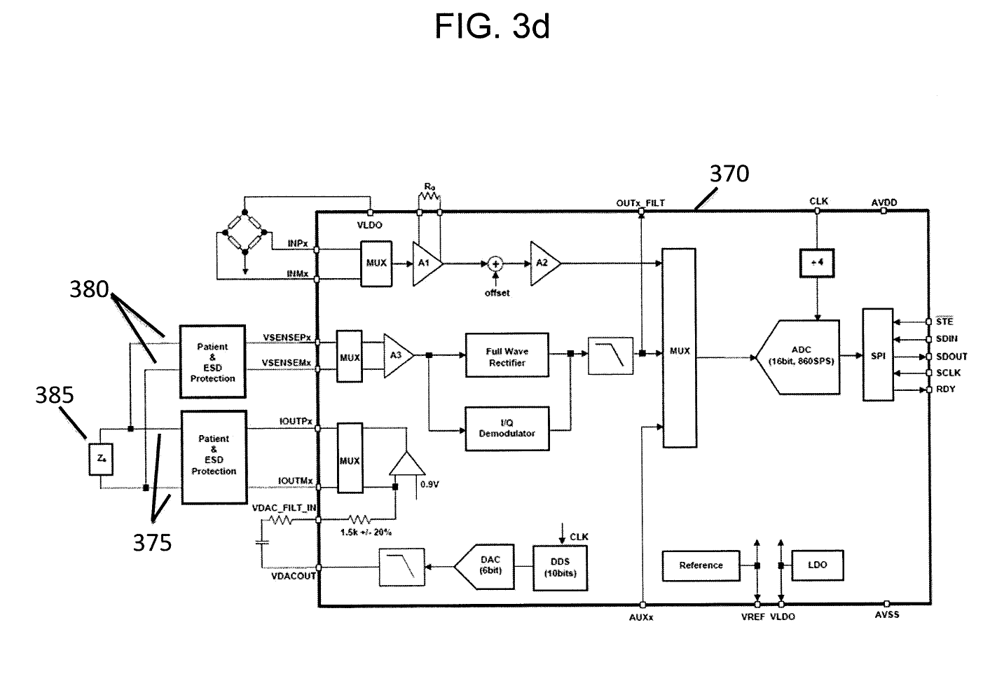

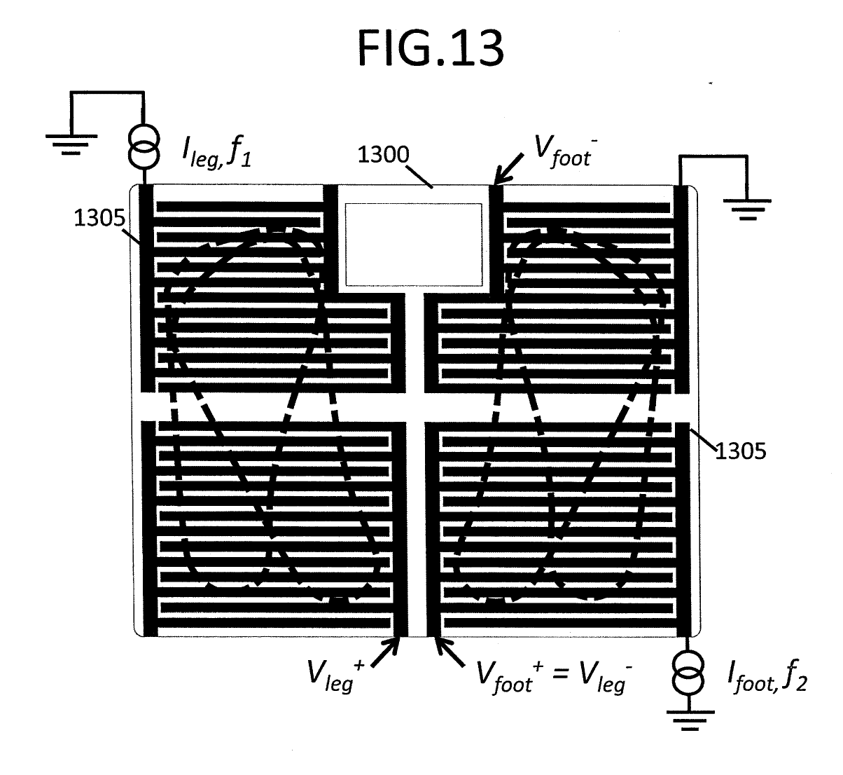

In a specific example, low security biometrics includes estimated weight (e.g., a weight range), and a toe tap on the FUI. Example medium security biometrics includes one or more the low security biometrics in addition to length and/or width of the user's foot, and/or a time of day or location of the scale. For example, as illustrated by FIGS. 2a and 13 and discussed with regard to FIG. 3c, the scale includes impedance electrodes that are interleaved and engage the feet of the user. The interleaved electrodes assist in providing measurement results that are indicative of the foot length, foot width, and type of arch. Further, a specific user, in some embodiments, may use the scale at a particular time of the day and/or authorize communication of data at the particular time of the day, which is used to verify identity of the user and authorize the communication. The location of scale, in some embodiments, is based on Global Positioning System (GPS) coordinates and/or a Wi-Fi code. For example, if the scale is moved to a new house, the Wi-Fi code used to communicate data externally from the scale changes. Example high security biometrics include one or more low security biometrics and/or medium security biometrics in addition to cardiogram characteristics and, optionally, a time of day and/or heart rate. Example cardiogram characteristics include a QRS complex, and QRS complex and P/T wave, BCG wave characteristics, and an ECG-to-BCG timing relationship, and combinations thereof.

In various embodiments, the user adjusts the table displayed above to revise the sensitivity values of each data type. Further, although the above-illustrated table includes a single sensitivity value for each data type, in various embodiments, one or more of the data types are separated into sub-data types and each sub-data type has a sensitivity value. As an example, the user-specific advertisement is separated into: prescription advertisements, external device advertisements, exercise advertisements, and diet plan advertisements. Alternatively and/or in addition, the sub-data types for user-specific advertisements include generic advertisements based on a demographic of the user and advertisements in response to scale collected data (e.g., advertisement for a device in response to physiologic parameters), as discussed further herein.

For example, weight data includes the user's weight and historical weight as collected by the scale. In some embodiments, weight data includes historical trends of the user's weight and correlates to dietary information and/or exercise information, among other user data. Body mass index data, includes the user's body mass index as determined using the user's weight collected by the scale and height. In some embodiments, similar to weight, body mass index data includes history trends of the user's body mass index and correlates to various other user data.

User-specific advertisement data includes various prescriptions, exercise plans, dietary plans, and/or other user devices and/or sensors for purchase, among other advertisements. The user-specific advertisements, in various embodiments, are correlated to input user data and/or scale-obtained data. For example, the advertisements include generic advertisements that are relevant to the user based on a demographic of the user. Further, the advertisements include advertisements that are responsive to scale collected data (e.g., physiological parameter includes a symptom or problem and advertisement is correlated to the symptom or problem). A number of specific examples include advertisements for beta blockers to slow heart rate, advertisements for a user wearable device (e.g., Fitbit®) to monitor heart rate, and advertisements for a marathon exercise program (such as in response to an indication the user is training for a marathon), etc.

Physician provided diagnosis/report data includes data provided by a physician and, in various embodiments, is in response to the physician reviewing the scale-obtained data. For example, the physician provided diagnosis/report data includes diagnosis of a disorder/condition by a physician, prescription medication prescribed by a physician, and/or reports of progress by a physician, among other data. In various embodiments, the physician provided diagnosis/reports are provided to the scale from external circuitry, which includes and/or accesses a medical profile of the user.

Suggestion data includes data that provides suggestions or advice for symptoms, diagnosis, and/or user goals. For example, the suggestions include advice for training that is user specific (e.g., exercise program based on user age, weight, and cardiogram data or exercise program for training for an event or reducing time to complete an event, such as a marathon), suggestions for reducing symptoms including dietary, exercise, and sleep advice, and/or suggestions to see a physician, among other suggestions. Further, the suggestions or advice include reminders regarding prescriptions. For example, based on physician provided diagnosis/report data and/or user inputs, the scale identifies the user is taking a prescription medication. The identification includes the amount and timing of when the user takes the medication, in some embodiments. The scale reminds the user and/or asks for verification of consumption of the prescription medication using the FUI.

As further specific examples, recent discoveries may align and associate different attributes of scale-based user data collected by the scale to different tools, advertisements, and physician provided diagnosis. For example, it has recently been discovered that atrial fibrillation is more directly correlated with obesity. The scale collects various user data and monitors weight and various components/symptoms of atrial fibrillation. In a specific embodiment, the scale recommends/suggests to the user to: closely monitor weight, recommends a diet, recommends goals for losing weight, and correlates weight gain and losses for movement in cardiogram data relative to arrhythmia. The movement in cardiogram data relative to arrhythmia, in specific embodiments, is related to atrial fibrillation. For example, atrial fibrillation is associated with indiscernible p-waves and beat to beat fluctuations. Thereby, the scale correlates weight gain/loss with changes in amplitude (e.g., discernibility) of a p-wave of a cardiogram (preceding a QRS complex) and changes in beat to beat fluctuations.

Further, the scale, in various embodiments, performs various security measures on the user data. For example, the scale performs encryption techniques on the data, has a hardware key and/or a software key. In various embodiments, the encryption scheme includes an asymmetric or symmetric key and the user data and/or the identifier is encrypted using an asymmetric or symmetric key cryptography. For example, the scale may not allow the ability to add additional applications or software to the circuitry (or the user may choose not to) and, thus, is more secure than if additional applications or software were added. In such embodiments, a symmetric key is used.

In various embodiments, a symmetric key is used by each scale using symmetric encryption. The key is randomly assigned by the scale instead of derived using a single key. A table of identifiers to keys is stored at the external circuitry (e.g., the second database). With symmetric encryption, the key and/or other data is encrypted by changing the data in a particular way. For example, the data is encrypted by shifting each letter or number by a number of places. Both the scale and the external circuitry know the symmetric key used to decode the data. Thereby, the symmetric key is a shared secret (e.g., piece of data known to the scale and to the external circuitry). The shared secret is known by the external circuitry before or at the start of the communication session.

Alternatively, an asymmetric key is used, which is sometimes referred to as a public key. With asymmetric key cryptography, there are two keys: a private key and a public key. The scale contains the only instance of the private key, which is kept secret, and the public key is provided to the external circuitry. Any message encrypted using the private key is decrypted using the matching public key and any message encrypted using the public key is decrypted by using the private key. The external circuitry contains a list of identifiers to public key mappings. The proof-of-identity supplied by the scale in the exchange is its identifier, as well as information to show authenticity and freshness of the message encrypted with its private key. To verify the user data, the external system looks up the public key and identifies that only the private key on the scale would create a message matching the known public key.

In various embodiments, the scale includes a hardware token that encrypts the data using a hardware security key generated using the hardware token. For example, the scale includes a hardware token and the external circuitry verifies authorization of the user data based on the hardware security key generated using the hardware token.

In accordance with a number of embodiments, the levels of verification and the security measures are provided by the scale based on the level of sensitivity of the data. For example, in response to a data communicate of a high sensitivity, the scale verifies identity of the user using a high level biometric, encrypts the data using a symmetric key encryption and adds a key using a hardware token. If the data is of a medium sensitivity, the scale verifies the identity of the user using a medium level biometric and encrypts the data using a symmetric key encryption. If the data is a low sensitivity, the scale verifies the identity of the user using a low level biometric. Embodiments are not limited to the specific example given and can include various combinations of biometric levels and other data security measures.

Using the scale as a hub to collect various user data and to communicate the user data to external circuitry, automatically and without user input, can reduce the time for a user to output various user data for correlation and processing. Further, as the scale is not accessible by other circuitry and/or may not include additional applications, the scale is less likely to be accessed by others, as compared to the user devices. For example, the scale accesses user data only in response to verifying the user using a scale-based biometric, in some embodiments. In various embodiments, the scale and user device (or external circuitry) can pair and/or otherwise communication in response to a verification or authorization of the communication, which can be based on confirming identification of the user device, that the same user is using the user device and the scale, and/or a scale-based biometric that is recognized. As a specific example, the user may be holding a cellphone in their hand while standing on the scale. The scale, using the processing circuitry and output circuit, outputs a command to the phone to vibrate. The scale detects the vibration frequency and timing (phase). This detected vibration frequency and timing can be used to securely identify the cellphone and/or to time synchronize the scale and the user device, as further described herein.

In various embodiments, the aggregated data from the scale and user devices is further processed and/or analyzed. For example, using the aggregated data, external circuitry, such as a standalone CPU and/or server CPU, medically assess the user, provides clinical indications, provides generic health information that correlates to the correlated data, and controls access to the various data, among other analysis. For example, using the aggregated data, the external circuitry determine cardio-related data. The cardio-related data includes physiological parameters, such as a cardiac output, a PWV, a revised BCG or ECG, pre-ejection period, stroke volume, arterial stiffness, respiration, and/or other parameters. Further, using the cardio-related data, the external circuitry derives clinical indication data. The clinical indication data, as used herein, is indicative of a physiological status of the user and can be used for assessment of a condition or treatment of the user. Example clinical indication data includes physiological parameters, risk factors, and/or other indicators that the user has a condition or could use a treatment. For example, the user is correlated with the condition or treatment by comparing the cardio-related data to reference information. The reference information, in various embodiments, includes a range of values of the cardio-related data for other users having the corresponding condition or treatment indicators. The other users are of a demographic background of the user, such that the reference information includes statistical data of a sample census.

For example, in specific embodiments, in response to the user standing on the scale, the scale transitions from the reduced-power mode of operation to the higher-power mode of operations and collects signals indicative of user's identity. In response to the transition, the scale collects signals indicative of cardio-physiological measurements (e.g., force signals). The processing circuitry 104 identifies a scale-based biometric using the collected signals and processes the signals to generate cardio-related physiologic data manifested as user data. Further, the processing circuitry validates user data, which includes data indicative of the user's identity and the cardio-related physiologic data, as concerning the user associated with the scale-based biometric. Optionally, the validation includes correlating the user data with a user ID in response to the validation. During, after, and/or before the identification of the scale-based biometric, the user device collects signals indicative of the user's identity and, therefrom, identifies authorization data corresponding to the user and user data. The user device communicates the user data, and, optionally the authorization data to the scale. In response to verifying the user data from the user device is correlated with the user, the scale aggregates the user data from the user device with the scale obtained data, encrypts the aggregated user data, and outputs the aggregated user data to external circuitry.

In accordance with a number of embodiments, as discussed further herein, the external circuitry provides additional health information to the user using the user data from the scale and user data from the user device. The user device (and/or the scale), for example, receives user input data that indicates the user is interested in additional (non-Rx) health information and various categories of interest. The categories of interest, in number of embodiments, include demographics of interest, symptoms of interest, disorders of interest, diseases of interest, drugs of interest, treatments of interest, etc. The user device and/or the scale further communicates the additional health information to another circuitry such that the user can print the additional health information.

FIG. 1b shows an example of a user-specific scale based enterprise system consistent with aspects of the present disclosure. As illustrated, user-specific scale based enterprise system includes at least one scale 118, the Internet (e.g., world-wide-web) 126, a standalone user CPU 119, and one or more user devices, such as a smartwatch 121, fitness tracking device, smartphone 122, smartbed, among other devices.

As previously discussed, the scale 118 collects highly user data, such as cardiogram data and data indicative of disorders and disease, and other user data, such as demographic information and weight. The scale 118 displays data, such as user weight, prompts or notification, and other information using a user interface 102, such as a FUI. The one or more user devices include devices that collect various user information, such as exercise data, food intake or liquid intake data, sleep data, cardiogram data, among other information. The standalone user CPU 119 includes a user device that include additional processing resources and/or a user display that is easier for the user to view data than the scale or other user devices. Thereby, the standalone user CPU 119, and other user devices form a robust graphical user interface (R-GUI) 123 for the user to view various data. In some embodiments, the standalone user CPU 119 includes a personal computer, a laptop, a tablet, and/or a smartphone. In various embodiments, the user devices can include implantable medical devices and/or other medical devices, such as a pacemaker that securely shares data to the scale.

In various embodiments, the scale 118 includes trigger data. The trigger data includes sensitivity values and/or combinations of different data values with user demographic information that indicates that the user has a risk for a condition, such as a disorder or disease. In response to the trigger data and the scale-obtained data or other user data from the other user devices indicating that the user has a risk for a condition, the scale prompts the user to determine if the user would like additional health information. Additionally, the scale can prompt the user to ask about other likely symptoms, prompt for further tests, such as breath hold, valsalva, etc. The prompt is display on the user display 102 of the scale 118 and/or using the R-GUI 123. For example, a synopsis of the prompt is displayed on the user display of the scale 118 and further information is display using the R-GUI 123 if the user is interested.

For example, the aggregated data from the scale 118 and the one or more user devices, in various embodiments, is compared to trigger data to determine if the user is at risk for a condition. The trigger data is stored directly on a memory circuit of the scale 118 and/or is stored on a memory circuit of the standalone user CPU 119 (and accessible by the scale). The trigger data includes values of various user data that indicate the user has a likelihood about a particular threshold of having and/or being at risk for a condition. In response to a match with the trigger data, the scale indicates a potential risk to the user and prompts the user to indicate if they would like more information. In response to the user indicating they would like further information, the enterprise system filters the user data for data correlated with the condition and filters the Internet for various data regarding the condition and/or matching the filtered user data. As previously described, the prompt can be provided using a FUI, a GUI, and/or voice input/output circuitry.

In response to the user selecting the prompt and indicating that they would like additional information, the scale 118 and/or standalone user CPU 119 filters the user data from the scale 118 and the other user devices 121, 122 and filters data from the Internet 126 to identify data that is relevant to the condition. In this manner, the enterprise system is used as a medical analytic driver that filters scale-obtained data, user device-obtained data, and data from the Internet to identify data related to the condition.

In response to the filter, the user and/or the scale, in various embodiments, are used to further assess the condition of the user and/or obtain additional information. The assessment includes the user assessing, using the scale user interface 102 or the R-GUI 123. For example, in response to the filter, the enterprise system identifies various addition information. The additional information include various generic health information, articles, blogs/forums or social groupings, and other data identified based on the filter of the Internet using the data that correlates with the condition and the trigger data. The user views the additional information using the interface 102 and/or R-GUI 123. The scale is used to further assess the condition of the user by performing additional tests (e.g., body-mass-index, QRS complex over time) and/or asking the user questions.

In various embodiments, the enterprise system provides a prompt to the user that indicates general information about the condition and the user has some risk for the condition. The prompt asks if the user would like more information and in response to the user requesting more information, the enterprise system provides the aggregated user data to a physician for review and to confirm the diagnosis. The physician is provided access to the user data using the internet 126 and/or external circuitry 124, such as server CPU that is accessible by the physician. In response to the physician confirming the diagnosis and/or correlation, the scale 118 is modified with the confirmed diagnosis.

For example, in a number of embodiments, the scale including the processing circuitry provides a number of questions to the user in response to input from the external circuitry. The scale can be used to provide questions to the user and obtain answers from the user. For example, the FUI can display a plurality of questions using the user display. Using user interaction by the user's foot, the FUI receives user inputs (e.g., answers) to each of the questions and, using the output circuit, stores the user inputs within a user profile associated with the user. For example, the FUI provides a number of questions in a question and answer session to identify symptoms, diagnosis, lifestyle data, family medical history, among other questions. The questions can be provided via a speaker component of the scale outputting computer generated natural voice (via a natural language interface), displaying the questions on the user display, and/or outputting the questions to another user-device. As previously described, the scale can (alternatively and/or in addition to a FUI or GUI) have a voice input/output circuitry that can obtain user's answers to questions via voice comments and inputs user information in response (e.g., a speaker component to capture voice sounds from the user and processing circuitry to recognize the voice commands and/or speech). The scale provides the input to the external circuitry 124 and the external circuitry 124 verifies or revises the risk identified.

The modification, in some embodiments, includes storing, on the scale 118, various correlation data (e.g., diagnosis data), adding additional devices and/or parameters to track (e.g., halter monitor, ECG tracking device, prescription drug titration, weight tracking and/or threshold values, exercise goals, stress test), and/or health information about the condition (e.g., articles), among other data. Furthermore, the standalone user CPU 119 of the enterprise system, in some embodiments is used to display various data to the user, such as generic health information, user-specific diagnosis data, blogs/forums of social groups, physician reports, and/or studies, among other information.

In various embodiments and environments, a single scale can be used by multiple different users. A subset or each of the different users can have user devices that can be synchronized to the scale and/or can be in communication and display scale-obtained data or aggregated user data via a GUI of the user device. The multiple users may synchronize their respective user devices to the common scale (or to multiple scales). Additionally, one or more of the user may have activated a service involving outputting aggregated data to the external circuitry for a variety of purposes, such as the social groups, physician reports, generic health information, etc., as described above in connections with various embodiments directed to filtering the user data for data correlated with the condition and filtering the Internet for various data regarding the condition and/or matching the filtered user data, and the scale can store an indication of the activation. The scale can selectively output aggregated data and/or portions thereof to different sources, such as the user device (for viewing on a GUI of the user device) and/or the external circuitry, responsive to identifying different biometrics to authorize the respective communication. The different biometrics can include a hierarchy of biometrics that correspond to communication of different levels of sensitivity of user data. In specific embodiments, the scale can verify that the user device has identified the user within a threshold period of time prior to synchronizing and/or communicating scale-obtained data.

Further, the scale can perform different levels of security on the user data prior to communicating externally from the scale. The different levels can be a function of the sensitivity of the user, with higher sensitivity user data having greater amounts of security techniques and/or resulting in a lower likelihood of identifying the user then lower sensitivity user data. Alternatively and/or in addition, the different levels of security can be implemented as a function of the identification of the external circuitry/user device and/or respective security measures of the external circuitry and/or user device. As an example, data output to a first user device which has previously been identified and verified by the user may have lower amounts of security performed than the same data output to a second user device which has not previously been identified. The data output to the second user device may, for example, be encrypted and cannot be viewed until the user enters a password and/or code. In other examples, the amount of security depends on security measures of the user device and/or external circuitry and/or accessibility of the user device and/or external circuitry. As an example, user data output to a first user device which is not connected to the Internet may have lower amounts of security performed than the same user data output to a second user device which is connected and is used by the user to browse the Internet (thus subject to security risks). As another example, user data output to server circuitry that is accessible from other circuitry for querying purposes may have higher security performed than user data output to a standalone external circuitry (or another server circuitry) that does not allow other devices to query the external circuitry (and/or has other security measures in place, such as firewalls, encryption on stored data, data masking, defense in depth, anti-virus techniques, hashing, intrusion detection systems, logging and auditing, multi-factor authentication, password and/or other authentication security, vulnerability scanners, physical security, virtual private network, timed access control, intrusion protection system, sandboxing, etc.) For example, a first external circuitry with a defense in depth system in place may have lower security measures performed on user data communicated thereto than a second external circuitry that has a firewall and anti-virus software.

The scale can be used in different setting and/or modes, such as a consumer mode, a professional mode, and a combination mode. A consumer mode includes or refers to a scale as used and/or operated in a consumer setting, such as a dwelling. As a specific example, a scale is located in a dwelling with five different people. Each of the five different people use the scale, and three of the five people utilize the scale to aggregate data from multiple devices and/or to output aggregated data to external circuitry (such as a user device, standalone CPU, and/or server CPU). Prior to providing a service to a user, the identity of the respective user is verified via the scale using scale-based biometric. As users in a consumer mode may be familiar with one another (e.g., live together), the identification of the user by the scale can be based on weight, body-mass-index, and/or other data. Although embodiments are not so limited and the identification can be based on other biometrics and/or passcodes.

In other instances the scale is used in a professional setting, such as a medical office, and/or in a professional mode. A professional mode includes or refers to an operation of the scale as used and/or operated in a professional setting, such as a doctor's office, exercise facility, nursing home, etc. In a professional mode, the scale is used by different users that may not be familiar with one another. The different users may have services with the professional to track and/or aggregate data from the user device (e.g., peripheral devices) to provide the professional with greater amounts of information. Similar to the consumer mode, the scale can selectively provide the services by verifying the identity of the user. The identification can include higher-level biometrics and/or identifications than the consumer mode. As a specific professional mode example, a scale is located at a doctor's office and is used to obtain data from multiple patients (e.g., 10 in a day, 500 in a year). When a patient checks-in, they stand on the scale and the scale-obtained data is output to external circuitry for document retention and/or other purposes. A subset (or all) of the patients have activated a service with doctor that corresponds with and/or includes acquisition and/or aggregation of data from a user device. For example, a user with atrial fibrillation can wear a smartwatch to track various cardio-related data during exercise and/or other periods of time and which is output to the scale at the doctor's office and/or other external circuitry. The scale, in the professional mode, may be used to obtain data from more users than a scale used in a consumer setting.

The scale can be in a combination consumer/professional mode. A combination consumer/professional mode includes or refers to a scale as used and/or operated in a consumer setting for purposes and/or uses by a professional, and/or in a professional setting for purposes and/or uses by the consumer (e.g., use by the consumer outside of the professional setting and/or in addition to). As a specific example, a scale is located at a user's dwelling and used by multiple family members. A first user of the family is diagnosed with a heart-related condition and the doctor may offer a service to review data from the scale (and optionally another user device) of the first user. When the other family members stand on the scale, the scale operates in the consumer mode. The other family members may or may not have the service activated for the doctor to review data (and/or other services involved in aggregating and outputting user data from the scale) and the scale operates via the consumer mode. When the first user that is diagnosed with heart-related condition stands on the scale, the scale recognizes the user and operates in a professional mode or a combination mode. For example, the scale outputs aggregated data (e.g., data obtained by the scale and data obtained by another user device) from the scale to external circuitry that is accessible by the doctor of the first user. As another specific example, a gym may offer gym subscriptions whose cost decreases as fitness of the user increases, which is determined using scale-obtained data. The cost maybe offset by insurance companies (e.g., health insurance) which offer contributions to a gym subscription if the user goes a threshold number of times in a month and/or based on other health factors.

Data provided to the user and/or the professional can default to be displayed on the FUI of the scale, the GUI of the user device, and/or a GUI of other external circuitry depending on the use of the scale. In a consumer mode and/or combination consumer/professional mode, data can default to display on the FUI of the scale. The defaulted display of data can be revised by the user providing inputs to display the data on the GUI of a user device or a GUI of another external circuitry (e.g., a standalone CPU) and/or automatically by the scale based on past scale-based actions of the user. As a specific example, a first user provides a user input to the scale to display data on the GUI of the user device multiple times (e.g., more than a threshold number of times, such as five times). In response, the scale adjusts the defaulted display of data and outputs data to the GUI of the user device. In a professional mode, the scale is not owned by the user. The user may be uninterested in synchronizing their user device with the professional's scale. The display of data may default to the GUI of the user device to display an option to synchronize, and/or to override the time-synchrony. Alternatively, the display of data may default to the FUI of the scale to display an option to synchronize and, responsive to user verification or authority to synchronize, defaults to display on the GUI of the user device. During the combination consumer/professional mode, portions of scale-obtained data for a particular user may default to display on external circuitry, such as a standalone or server CPU that is accessible by the professional.

FIG. 1c shows an example of a scale aggregating and communicating user data from various user devices, consistent with aspects of the present disclosure. As illustrated, a scale 118 is in communication with various user device 121, 122, 127, and a standalone CPU 109. The scale, illustrated by FIG. 1c includes the scale and the various circuitry illustrated and previously described in connection with FIG. 1a. The scale collects various user data that is sensitive to the users. The user devices 121, 122, 127 further automatically collect various user data, such as sleep data, cardiogram data, exercise data, heart rate data, and food/liquid intake data. In some embodiments, various user data is manually entered by the user to the standalone CPU 109, the scale 118, and/or a smartphone 122. Such data includes user demographic data, food/liquid intake data, and/or sleep data, in some embodiments.

The various user devices 121, 122, 127, and the standalone CPU 109 communicate various user data to the scale 118. The scale 118 aggregates the user data and secures the aggregated user-data prior to sending to data to external circuitry, such as the standalone CPU 109 and/or server CPU. For example, in response to the user standing on the scale, the scale transitions from a reduced power-consumption mode of operation 129 to at least one higher power-consumption mode of operation 131. At 132, the scale collects signals indicative of an identity of the user and cardio-physiological measurements (e.g., force signals) by engaging the user with electrical signals and, therefrom, collecting the signals. Further, at 132, the processing circuitry of the scale, processes the signals obtained by the data-procurement circuitry while the user is standing on the platform and generates, therefrom, cardio-related physiologic data corresponding to the collected signals.

At 133, the processing circuitry of the scale identifies a scale-based biometric of the user using the collected signals and validates the user data, which includes the data indicative of the users identity and the generated cardio-related physiologic data, as concerning the user associated with the scale-based biometric. In various embodiments, the scale receives user data from the user device. In some embodiments, the scale authorizes the communication in response to a dual-authorization. For example, optionally, at 134, the scale waits for dual-authorization. The dual-authorization includes the communication activation circuit of the scale receiving a scale-based biometric corresponding to a specific user and authorization data from the user device corresponding to the same specific user.

The user devices, as previously discussed, includes a device, including processing circuitry, configured to collect various signals from the user. In various embodiments, one or more of the user devices are configured to operate in multiple modes. For example, the user device can wait for user authorization data from the user. The user authorization data, as previously discussed, includes the user entering a password or finger print to the user device to transition the user device from a reduced-power mode of operation to a higher-power mode of operation. Alternatively and/or in addition, the user authorization data includes a password, pass code, and/or biometric data obtained in response to the user accessing the specific functionality (e.g., an application) of the user device capable of generating cardio-related physiologic data and/or other user data.

In response to the authorization data, the user device collects signals, such as signals indicative of the cardio-physiologic data, exercise data, sleep data, and generates therefrom the user data. Further, at 134, the user device activates the communication by outputting the authorization data to the scale. The authorization data is output concurrently, during, and/or after the collection of signals. Alternatively, the authorization data is output as a portion of the user data and the scale authorizes the data based on the authorization data. In various embodiments, the user device and scale can time synchronize prior to obtaining the user data, as further described herein.

At 137, in response to the communication of user data to the scale, the scale aggregates the user data from the user devices with scale obtained user data. In various embodiments, the aggregation includes the scale correlating and storing the data obtained by the user device and the scale with a user profile of the user.

At 141, the scale secures the data. Securing the data, as previously discussed, includes various verification of the identity of the user (e.g., different biometrics to authorize different sensitivity levels), encryption schemes, software keys, hardware token keys, among other techniques. The scale outputs the user data, as aggregated, to external circuitry in response to the authorization and security.

In a number of embodiments, the external circuitry provides (e.g., determines) clinical indication data by processing the aggregated user data. The clinical indication data, in various embodiments, include physiologic parameters (such as PWV, BCG, respiration, arterial stiffness, cardiac output, pre-ejection period, stroke volume), diagnosis, conditions, and risk factors, among other health information.

In various related embodiments, the external circuitry determines additional health information and provides the additional health information for display to the user. The additional health information is indicative of the clinical indication data and correlates to the categories of interest provided by the user. The categories of interest are provided at a different time, the same time and/or from the scale. In various embodiments, the additional health information is based on historical user-data. For example, the additional health information (e.g., a table) provided include a correlation to the category of interest and the user data over time.

In various embodiments, the system includes additional user devices and/or other body accessories. For example, the scale receives data from a plurality of user devices and/or other body accessories. In this way, the scale is used as a hub for collecting and correlating data corresponding to a user. For example, the data can include fitness data, cardio-related data, user input data (e.g., calorie counts/food intake, drug dosage, treatment, sleep schedule), sleep schedule (e.g., directly input from a smartbed and/or other body accessory), among other data. The scale collects the various data and correlates the data with a user profile corresponding with the user. In various embodiments, the data from one of the user devices may conflict with data obtained by the scale. In such instances, the data obtained by the scale is used and the data from the user device is discarded. That is, the data from the scale is the default data as the scale may include greater processing resources and/or obtain higher quality signals than the user device.

The scale can time synchronize with the other devices prior to the scale and other device obtaining the user data, in various specific embodiments. When using data from both the scale and another device, time-based (e.g., phase) inaccuracies between user data sets from the other device and the scale can impact assimilation and/or combined use of the two sets of user data. For example, lack of time synchrony can cause issues such as cardiac parameters from each device not coordinating, and/or being inaccurate, and/or not identifying the correct data to output. For example, a user exercises while wearing a user device (e.g., a wearable device) that monitors one or more physiological parameters, and the user device outputs the physiological parameters to a scale for further processing. The time (e.g., phase) used by the user device can cause a resulting physiological parameter (e.g., waveform) to be inaccurate. The scale and the user device (or other user devices) can be time-synchronized based on the frequency and/or timing (e.g., phase) of signals or waveforms. Time-synchronizing includes or refers to synchronizing two waveforms (e.g., signals from the scale and the user device) based on a frequency and a timing, sometimes referred to as “a phase angle”. In specific embodiment, time-synchronized waveforms have the same frequency and same phase angle with each cycle and/or share repeating sequences of phase angles over consecutive cycles.

The following is a specific example of a user device time-synchronizing with a scale prior to obtaining user data. While the user is standing on the scale, the scale recognizes a nearby user device (e.g., within a threshold) and prompts the user to pair the user device and scale. The user authorizes the pairing (e.g., selects an icon on the FUI or otherwise provides an indication of an interest) by providing an indication of interest to the scale (e.g., select an icon, provide a voice command, or perform an action). In specific embodiments, the user device and scale can be time-synchronized by tapping the user device, such as a wearable device, cellphone, and/or tablet to the scale. The scale synchronizes via strain gauges of the scale and accelerometer of the user device, as previously described. In other specific embodiments, the scale provides a command to the user device, which is placed on the scale and/or tapped on the scale, the scale detects the vibration frequency and timing (e.g., phase). This can be used to give secure identification and time synchronization, as previously described.

In a number of specific embodiments, the user activates a time-synchronization service/feature of the scale. For example, the user stands on the scale and identifies the user device including how to synchronize the two devices, using a user interface (e.g., FUI of the scale, external GUI in communication, etc.) The scale authorizes the communication and/or the synchronization by recognizing the user using a scale-based biometric and based on authorization data from the user device, in some specific embodiments. In response to the synchronization, the scale outputs a message requesting a time value from the user device. The user device, in response to the message, outputs a response message with an indication of the time value. The response message can include the user device vibrating (at a respective frequency and timing). The scale detects the vibration at a frequency and timing, and can determine the vibration frequency and timing. The determined vibration frequency and timing can be used to time-synchronize the scale with the user device based on a time difference. A time difference between the scale and the user device can include a difference in relative time (e.g., phase) according to the scale and relative time (e.g., phase) according to the user device. The scale can time-synchronize by outputting a message to the user device to adjust its timing based on the time difference and/or to match the timing of the scale.

As previously described, the time-synchronization can occur responsive to a user dropping and/or tapping the user device on the scale. The user device may include a built-in accelerometer and the user dropping or tapping the user device on the platform of the scale (with or without standing on the scale) can activate the time-synchrony. In various embodiments, the time-synchrony is activated in response to the user device being within a threshold distance from the scale. In other embodiments, the user is standing on the scale and/or within a threshold distance, and the scale outputs a messaged to the user device to vibrate to trigger the time-synchronization, as previous discussed. Further, via NFC, Bluetooth, and/or wireless communication, the time-synchrony can occur through direct communication between the scale and the user device. In some specific embodiments, the time-synchrony occurs in response to verification that the user device (and/or the scale) has recognized the user within a threshold period of time. The verification can be used to mitigate or prevent accidental synchronization and can be used in combination with a user dropping or tapping the user device on the scale and/or the user device being within a threshold distance from the scale.

In other specific embodiments, the scale time-synchronizes with the user device by docking the user device with the scale and/or via acoustic sounds. For example, the user device may be a remote user-physiologic device that includes a photoplethy configured to obtain a photoplethysmogram. The photoplethy can be time-synchronized by docking (e.g., placing on the platform and/or connecting) the remote user-physiologic device with the scale and using a light source of the scale to flash a pattern to calibrate the photoplethy (e.g., flashing LED lights via one or more LEDs embedded in the platform of the scale). Further, the user device can be acoustically calibrated by outputting sounds from the platform (e.g., “pips” and “chirps”).

The scale can include a mechanical mass that can be triggered by the user device to calibrate the system. In response to a command from the user device, for example, a mechanical input is input to circuitry of the scale using the mechanical mass. The scale can pick apart the mechanical input separately from a cardiac parameter (e.g., BCG) and use the mechanical input to measure a phase latency of the system.

FIG. 1d shows an example of filtering data from a user-specific scale-based enterprise system, consistent with aspects of the present disclosure. As illustrated the user-specific scale based enterprise system includes a scale 118, standalone CPU 109, and various user devices (e.g., smartwatch 121, smartphone 122, and smartcup 127). The user-specific scale based enterprise system is used as a medical analytic driver that provides data by filtering the user data based on trigger data and filtering data from the internet based on the filtered user data and trigger data.

The scale is configured to monitor signals and/or data indicative of physiologic parameters of the user while the user is standing on the platform (e.g., collect scale-based/obtained data 143). The user devices further monitors signals and/or data indicative of physiologic parameters of the user. Both the scale and the user device collect user data of varying user sensitivities. For example, the scale 118 collects user data, such as cardiogram data and data indicative of disorders and disease, and other user data, such as demographic information and weight. The user devices collect user data such as exercise data, food intake or liquid intake data, sleep data, cardiogram data, among other information.

The standalone user CPU 109, and other user devices form a R-GUI for the user to view various data. In some embodiments, the standalone user CPU 109 includes a personal computer, a laptop, a tablet, and/or a smartphone. Further, the scale 118 includes a GUI, such as a FUI. In various embodiments, using the scale-based and/or obtained data 143, such as user demographic data, various reports or dashboards are displayed using the FUI and/or the GUI. The reports/dashboards includes displays of various scale-obtained parameter values and/or progress. As an example, a report is provided that illustrates the user's loss of weight over the last two months and is displayed on the R-GUI.

As previously discussed, the scale (and/or standalone CPU 109) includes trigger data. The scale-based/obtained user-specific data 143 is compared to the trigger data to determine if the user has or is at risk for a condition. In various embodiments, the aggregated data from the scale and the one or more user devices is compared to the trigger data, although embodiments are not so limited. The trigger data includes user data values and/or combinations of different data values with user demographic information that indicates that the user has a risk for a condition, such as a disorder or disease. In response to the trigger data and the scale-obtained data or other user data from the other user devices indicating that the user has a risk for a condition, the scale prompts the user to determine if the user would like additional health information. The prompt is displayed on the user display 102 of the scale 118 and/or using the R-GUI. For example, a synopsis of the prompt is displayed on the user display of the scale 118 and further information is displayed using the R-GUI if the user is interested.

In response to the user selecting the prompt indicating they are interested in additional information, the scale 118 and/or standalone user CPU 109 filters the user data from the scale 118 and the other user devices 121, 122, 127 and filters data from the Internet 144 to identify data that is relevant to the condition using a scale-enterprise filter circuitry, at block 146. For example, first the user data is filtered to identify a subset of the user data that is relevant to the condition, such as based on the trigger data. The subset of user data and trigger data is used to filter data from the Internet 144, in various embodiments. The filter results in various additional health information identified by searching the Internet 144 based on the filters, such as generic health information related to the condition, social groupings, additional symptoms, additional tests or parameters to perform, devices and/or products related to the condition, blogs, studies, etc.

In response to the filter identifying various health information, the user and/or the scale 118, in various embodiments, are used to further assess the condition of the user and/or obtain additional information. For example, at block 147, the user further assesses the condition by viewing the various health information on the FUI of the scale 118 and/or the R-GUI. In various embodiments, the display of the data is discerned by the scale, as discussed further herein. The scale is used to further assess the condition of the user by performing additional tests (e.g., body-mass-index, QRS complex over time) and/or asking the user questions, at block 148. For example, the scale can prompt the user to perform additional tests, such as breath hold, valsalva, etc.

Alternatively and/or in addition, the enterprise system provides a prompt to the user that indicates general information about the condition and the user is indicating some risk for the condition. The prompt asks if the user would like more information and in response to the user requesting more information, the enterprise system provides the aggregated user data to a physician for review and to confirm the diagnosis, at block 149. The physician is provided access to the user data using the internet and/or external circuitry, such as server CPU that is accessible by the physician. In response to the physician confirming the diagnosis and/or correlation, the scale 118 is modified with the confirmed diagnosis, at block 151. In specific aspects, the scale can incorporate a web server (URL) that allows secure, remote access to the collected data. For example, the secure access can be used to provide further analysis and/or services to the user.

The modification, in some embodiments, includes storing, on the scale 118, various correlation data (e.g., diagnosis data), adding additional devices and/or parameters to track (e.g., halter monitor, ECG tracking device, prescription drug titration, weight tracking and/or threshold values, exercise goals, stress test), and/or health information about the condition (e.g., articles), among other data. Furthermore, the standalone user CPU 109 of the enterprise system, in some embodiments is used to display various data to the user, such as generic health information, user-specific diagnosis data, blogs/forums of social groups, physician reports, and/or studies, among other information.

In accordance with various embodiments, the FUI of the scale is used to provide portions of the user data, diagnosis data (e.g., scale-obtained physiological data), generic health information, and/or other feedback to the user. In some embodiments, the scale 118 includes a display configuration filter (e.g., circuitry and/or computer readable medium) configured to discern the data to display to the user and displays the portion. The display configuration filter discerns which portions of the data to display to the user on the FUI based on various user demographic information (e.g., age, gender, height, diagnosis) and the amount of data. For example, the generic health information identified from the filter 145 may include an amount of data that if all the data is displayed on the FUI, the data is difficult for a person to read and/or uses multiple display screens.

The display configuration filter discerns portions of the data to display using the scale user interface 102, such as synopsis of the generic health information (or user data or feedback) and an indication that additional data is displayed on another user device, and other portions to display on the other user device (e.g., the R-GUI). The other user device is selected by the scale (e.g., the filter) based on various communications settings. The communication settings include settings such as user settings (e.g., the user identifying user devices to output data to), scale-based biometrics (e.g., user configures scale, or default settings, to output data to user devices in response to identifying scale-based biometrics), and/or proximity of the user device (e.g., the scale outputs data to the closest user device among a plurality of user devices and/or in response to the user device being within a threshold distance from the scale), among other settings. For example, the scale determines which portions of the used data, clinical indication, generic health information and/or other feedback to output and outputs the remaining portion of the user data, clinical indication, generic health information and/or other feedback to a particular user device based on user settings/communication authorization (e.g., what user devices are authorized by the user to receive particular user data from the scale), and proximity of the user device to the scale. The determination of which portions to output is based on what type of data is being displayed, how much data is available, and the various user demographic information (e.g., an eighteen year old is able to see better than a fifty year old).

In accordance with a number of embodiments, the enterprise system provides additional health information to the user. The R-GUI and/or FUI, for example, receives user input data that provides an indication that the user is interested in additional (non-Rx) health information and various categories of interest. The categories of interest include demographics of interest, symptoms of interest, disorders of interest, diseases of interest, drugs of interest, treatments of interest, etc. The additional health information is derived and provided to the user.

For example, in a number of embodiments, the GUI and/or FUI provides a number of questions to the user. In various embodiments, the questions include asking the user if the user is interested in additional health information and if the user has particular categories of interest. In various embodiments, the categories of interest include a set of demographics, disorders, diseases, and/or symptom that the user is interested, and/or other topics. The additional health information includes a table that corresponds to the categories of interest and/or corresponds to the physiological parameter and/or clinical indications determined without providing any specific values and/or indication related to the physiological parameter, among other data. The user is provided the additional health information by the GUI and/or FUI.

The additional health information is generated, in various embodiments, by comparing the categories of interest to the aggregated user data. In various embodiments, the correlation/comparison include comparing statistical data of a sample census pertinent to the categories of interest and at least one physiological parameter determined using the aggregated user data. The statistical data of a sample census includes data of other users that are correlated to the categories of interest. In such instances, the additional health information includes a comparison of data measured while the user is standing on the platform 101 and data measured using the user device to sample census data (e.g., may contain Rx information). In other related embodiments, the correlation/comparison includes comparing statistical data of a sample census pertinent to the categories of interest and values of the least one physiological parameter of the sample census. In such instances, the additional health information includes average physiological parameter values of the sample census that is set by the user, via the categories of interest, and may not include actual values corresponding to the user (e.g., may not contain Rx information). As previously discussed, the scale can include voice input/output circuitry to receive the categories of interest from the user via voice commands.

Various categories of interest, in accordance with the present disclosure, include demographics of the user, disorders, disease, symptoms, prescription or non-prescription drugs, treatments, past medical history, family medical history, genetics, life style (e.g., exercise habits, eating habits, work environment), among other categories and combinations thereof. In a number of embodiments, various physiological factors are an indicator for a disease and/or disorder. For example, an increase in weight, along with other factors, can indicate an increased risk of atrial fibrillation. Further, atrial fibrillation is more common in men. In some instances, symptoms of a particular disorder are different for different categories of interest (e.g., symptoms of atrial fibrillation can be different between men and women). For example, in women, systolic blood pressure is associated with atrial fibrillation. In other instances, sleep apnea may be assessed via an ECG and is correlated to weight of the user. Furthermore, various cardiac conditions are assessed using an ECG. For example, atrial fibrillation can be characterized and/or identified in response to a user having no or fibrillating p-waves, no QRS complex, and no baseline/inconsistent beat fluctuations. Atrial flutter, by contrast, can be characterized by having no p-wave, variable heart rate, having QRS complexes, and a generally regular rhythm. Ventricular tachycardia (VT) can be characterized by a rate of greater than 120 beats per minute, and short or broad QRS complexes (depending on the type of VT). Atrio-Ventricular (AV) block can be characterized by PR intervals that are greater than normal (e.g., a normal range for an adult is generally 0.12 to 0.20 seconds), normal-waves, QRS complexes can be normal or prolong shaped, and the pulse can be regular (but slow at 20-40 beats per minute). For more specific and general information regarding atrial fibrillation and sleep apnea, reference is made herein to https://www.clevelandclinicmeded.com/medicalpubs/diseasemanagement/cardiology/atri al-fibrillation/and http://circ.ahajournals.org/content/118/10/1080.full, which are fully incorporated herein for its specific and general teachings. Further, other data and demographics that are known and/or are developed can be added and used to derive additional health information.

For example, the categories of interest for a particular user can include a change in weight, age 45-55, and female. The scale obtains raw data, including user weight, using the data-procurement circuitry and the remote user-physiologic device obtains raw data and the categories of interest from the user. The scale outputs the raw data to the remote user-physiologic device 109 or the remote user-physiologic device 109 outputs signals indicative of cardio-related physiologic data (responsive to activation of the communication). The enterprise system correlates the categories of interest to the various raw data and derives non-prescription health information therefrom. Further, the enterprise system, over time, historically collects and correlates the categories of interest of the user and data from the data-procurement circuitry. The enterprise system, in various embodiments, sends the data to a physician and/or non-Rx health information to the user (to print and/or otherwise view).

In various embodiments, the enterprise system is used to group users of the scale. A social group, as used herein, includes grouping of a set of scale users based on the aggregated user data. In some embodiments, the social groups are intra scale and/or intra scale.

Intra scale social groups includes users that use a single scale. For example, the scale is configured to collected data from multiple users. As a specific example, a scale is used by a family training for a marathon. Each member of the family uses the scale to track various physiological parameters, include cardiogram related characteristics, recovery parameters, weight, body-mass-index, and exercise results. With intra scale social groups, the users are using the same scale and, thereby, have a familiarity with one another. Thereby, the user's identities are disclosed in the social groups, in some embodiments. The family is grouped into an intra scale social grouping and provided with alerts when reports of progress and/or rankings are available for the family. Further, one or more of the users are provided an alert in response to user-configured thresholds, such as a weight threshold.

Inter scale groups include users that use different scales. For example, the scale communicates user data to an external circuitry, such as a server CPU that pools the user data and identifies correlations between the user data. The social groups are identified automatically by the external circuitry based on the user data. The social groups are based on demographics, user goals, symptoms, physiological parameter values, diagnosis, prescription drug usage, lifestyle habits, medical history, family medical history, and a combination thereof. In specific embodiments, the external circuitry groups user data based on fitness goals (current or historical), demographic information, and scale-obtained data. The external circuitry analyzes the pooled user data from the plurality of scales to identify various correlations between users and dynamically updates the first database over time. Based on the correlated user data sets, the external circuitry identifies various user data sets with correlation and provides users of the correlated data sets access to a social group. The correlation can include demographics, values of user data, user goals, risks, diagnosis, condition, etc.

Example reports/dashboards indicates progress, others successes and failures, new diagnosis information or treatments, and other data. In various embodiments, the external circuitry uses the user inputs to the forum, blogs, and/or webpage to update the user-specific knowledge database.

In various embodiments, in response to the social group, the external circuitry outputs a prompt that notifies the respective user of the availability of a social group and generates a way to access the social group, such as generated a new blog, form, and/or page of a social network. Alternatively and/or in addition, the access to the social group includes accessing (e.g., via the FUI or GUI) reports and/or dashboards about user-data.

Further, the user data, in some embodiments, does not identify the user. For example, with inter scale social groups, the users are not using the same scale and, thereby, may not have familiarity with one another. Thereby, the users' identities are not disclosed in the inter scale social groups, in some embodiments. For example, the scale removes portions of the user data that identifies the user, adds an identifier indicative of the user and the scale to the user data, optionally encrypts at least a portion of the user data, and outputs at least a portion of the user data to external circuitry. The external circuitry processes the collected user data by replacing the identifier with an alias ID, storing the user data with the alias ID in a first database that has pooled user data from a plurality of scales, and storing identification of the respective scale and user that corresponds to the alias ID in a second database. An alias ID, as used herein, is data that is independent of the identifier (e.g., not invertible back to the identifier).

The data provided to the social groups may not be the entire set of user data. For example, in accordance with various embodiments, there is a selective relationship between the social group, the number of users in the group, and one or more of the following: level of familiarity between users, level of familiarity of users with physiological data, level of interest in physiological data, and a level of complexity of the data displayed. A level of familiarity between users includes knowledge of identity of the users and/or interactions between the users. A level of familiarity of users with physiological data include technical knowledge of the users regarding physiologic data. A level of interest in physiological data includes interest of the user in more information related to physiologic data. And a level of complexity of the data displayed includes the technical complexity of the subset of user data provided/displayed to the social group.

For example, the social group includes an intra scale social group of a family trying to lose weight. The members of the social group are familiar with one another but do not have significant knowledge/background regarding physiological data. However, the users are interested in how weight loss is correlated with physiological data. In various embodiments, the social group is provided access, such as reports, to user data that identifies each user of the group and includes a general correlation of a cardiogram data with weight loss/gains. The users are not provided with specific data, such as BCG and/or PWV that is more complex.

As another example, the social group includes an inter scale professional social group that includes a physician and a number of users that do not know one another. The members of the social group are not familiar with another and the number of users do not have significant knowledge/background regarding physiological data. However, the physician does. In various embodiments, the social group is provided access, such as reports, to user data that does not identify each user of the group and includes specific data, such as BCG and/or PWV that is more complex. The physician, in such embodiments, may be provided the identification of the user and can explain the more complex data.

The above described social group and various levels can be used in combination with various features described herein, such as encryption of the data. Further, portions of the data is displayed using a FUI of the scale. The scale discerns which portions to display based on the above-described levels and/or the sensitivity values, as previously described.

The remaining figures illustrate various ways to collect the physiologic data from the user, electrode configurations, and alternative modes of the processing circuitry 104. For general and specific information regarding the collection of physiologic data, electrode configurations, and alternative modes, reference is made to U.S. patent application Ser. No. 14/338,266 filed on Oct. 7, 2015, which is hereby fully incorporated by references for its teachings.

FIG. 1e shows current paths 100 through the body of a user 105 standing on a scale 110 for the IPG trigger pulse and Foot IPG, consistent with various aspects of the present disclosure. Impedance measurements 115 are measured when the user 105 is standing and wearing coverings over the feet (e.g., socks or shoes), within the practical limitations of capacitive-based impedance sensing, with energy limits considered safe for human use. The measurements 115 can be made with non-clothing material placed between the user's bare feet and contact electrodes, such as thin films or sheets of plastic, glass, paper or wax paper, whereby the electrodes operate within energy limits considered safe for human use. The IPG measurements can be sensed in the presence of calluses on the user's feet that normally diminish the quality of the signal.

As shown in FIG. 1d, the user 105 is standing on a scale 110, where the tissues of the user's body will be modeled as a series of impedance elements, and where the time-varying impedance elements change in response to cardiovascular and non-cardiovascular movements of the user. ECG and IPG measurements sensed through the feet can be challenging to take due to small impedance signals with (1) low SNR, and because they are (2) frequently masked or distorted by other electrical activity in the body such as the muscle firings in the legs to maintain balance. The human body is unsteady while standing still, and constant changes in weight distribution occur to maintain balance. As such, cardiovascular signals that are measured with weighing scale-based sensors typically yield signals with poor SNR, such as the Foot IPG and standing BCG. Thus, such scale-based signals require a stable and high quality synchronous timing reference, to segment individual heartbeat-related signals for signal averaging to yield an averaged signal with higher SNR versus respective individual measurements.

The ECG can be used as the reference (or trigger) signal to segment a series of heartbeat-related signals measured by secondary sensors (optical, electrical, magnetic, pressure, microwave, piezo, etc.) for averaging a series of heartbeat-related signals together, to improve the SNR of the secondary measurement. The ECG has an intrinsically high SNR when measured with body-worn gel electrodes, or via dry electrodes on handgrip sensors. In contrast, the ECG has a low SNR when measured using foot electrodes while standing on said scale platforms; unless the user is standing perfectly still to eliminate electrical noise from the leg muscles firing due to body motion. As such, ECG measurements at the feet while standing are considered to be an unreliable trigger signal (low SNR). Therefore, it is often difficult to obtain a reliable cardiovascular trigger reference timing when using ECG sensors incorporated in base scale platform devices. Both Inan, et al. (IEEE Transactions on Information Technology in Biomedicine, 14:5, 1188-1196, 2010) and Shin, et al. (Physiological Measurement, 30, 679-693, 2009) have shown that the ECG component of the electrical signal measured between the two feet while standing was rapidly overpowered by the electromyogram (EMG) signal resulting from the leg muscle activity involved in maintaining balance.

The accuracy of cardiovascular information obtained from weighing scales is also influenced by measurement time. The number of beats obtained from heartbeats for signal averaging is a function of measurement time and heart rate. Typically, a resting heart rates range from 60 to 100 beats per minute. Therefore, short signal acquisition periods may yield a low number of beats to average, which may cause measurement uncertainty, also known as the standard error in the mean (SEM). SEM is the standard deviation of the sample mean estimate of a population mean. Where, SE is the standard error in the samples N, which is related to the standard error or the population S. The following is an example SE for uncorrelated noise:

$$SE = \frac{S}{\sqrt{N}}$$

For example, a five second signal acquisition period may yield a maximum of five to eight beats for ensemble averaging, while a 10 second signal acquisition could yield 10-16 beats. However, the number of beats available for averaging and SNR determination is usually reduced for the following factors; (1) truncation of the first and last ensemble beat in the recording by the algorithm, (2) triggering beats falsely missed by triggering algorithm, (3) cardiorespiratory variability, (4) excessive body motion corrupting the trigger and Foot IPG signal, and (5) loss of foot contact with the measurement electrodes.

Sources of noise can require multiple solutions for SNR improvements for the signal being averaged. Longer measurement times increase the number of beats lost to truncation, false missed triggering, and excessive motion. Longer measurement times also reduce variability from cardiorespiratory effects. If shorter measurement times (e.g., less than 30 seconds) are desired for scale-based sensor platforms, sensing improvements need to tolerate body motion and loss of foot contact with the measurement electrodes.

The human cardiovascular system includes a heart with four chambers, separated by valves that return blood to the heart from the venous system into the right side of the heart, through the pulmonary circulation to oxygenate the blood, which then returns to the left side of the heart, where the oxygenated blood is pressurized by the left ventricles and is pumped into the arterial circulation, where blood is distributed to the organs and tissues to supply oxygen. The cardiovascular or circulatory system is designed to ensure oxygen availability and is often the limiting factor for cell survival. The heart normally pumps five to six liters of blood every minute during rest and maximum cardiac output during exercise increases up to seven-fold, by modulating heart rate and stroke volume. The factors that affect heart rate include autonomic innervation, fitness level, age and hormones. Factors affecting stroke volume include heart size, fitness level, contractility or pre-ejection period, ejection duration, preload or end-diastolic volume, afterload or systemic resistance. The cardiovascular system is constantly adapting to maintain a homeostasis (set point) that minimizes the work done by the heart to maintain cardiac output. As such, blood pressure is continually adjusting to minimize work demands during rest. Cardiovascular disease encompasses a variety of abnormalities in (or that affect) the cardiovascular system that degrade the efficiency of the system, which include but are not limited to chronically elevated blood pressure, elevated cholesterol levels, edema, endothelial dysfunction, arrhythmias, arterial stiffening, atherosclerosis, vascular wall thickening, stenosis, coronary artery disease, heart attack, stroke, renal dysfunction, enlarged heart, heart failure, diabetes, obesity and pulmonary disorders.

Each cardiac cycle results in a pulse of blood being delivered into the arterial tree. The heart completes cycles of atrial systole, delivering blood to the ventricles, followed by ventricular systole delivering blood into the lungs and the systemic arterial circulation, where the diastole cycle begins. In early diastole the ventricles relax and fill with blood, then in mid-diastole the atria and ventricles are relaxed and the ventricles continue to fill with blood. In late diastole, the sinoatrial node (the heart's pacemaker) depolarizes then contracting the atria, the ventricles are filled with more blood and the depolarization then reaches the atrioventricular node and enters the ventricular side beginning the systole phase. The ventricles contract and the blood is pumped from the ventricles to arteries.

The ECG is the measurement of the heart's electrical activity and is described in five phases. The P-wave represents atrial depolarization, the PR interval is the time between the P-wave and the start of the QRS complex. The QRS wave complex represents ventricular depolarization. The QRS complex is the strongest wave in the ECG and is frequently used as a timing reference for the cardiovascular cycle. Atrial repolarization is masked by the QRS complex. The ST interval represents the period of zero potential between ventricular depolarization and repolarization. The cycle concludes with the T-wave representing ventricular repolarization.

The blood ejected into the arteries creates vascular movements due to the blood's momentum. The blood mass ejected by the heart first travels headward in the ascending aorta and travels around the aortic arch then travels down the descending aorta. The diameter of the aorta increases during the systole phase due to the high compliance (low stiffness) of the aortic wall. Blood traveling in the descending aorta bifurcates in the iliac branch which transitions into a stiffer arterial region due to the muscular artery composition of the leg arteries. The blood pulsation continues down the leg and foot. Along the way, the arteries branch into arteries of smaller diameter until reaching the capillary beds where the pulsatile blood flow turns into steady blood flow, delivering oxygen to the tissues. The blood returns to the venous system terminating in the vena cava, where blood returns to the right atrium of the heart for the subsequent cardiac cycle.

Surprisingly, high quality simultaneous recordings of the Leg IPG and Foot IPG are attainable in a practical manner (e.g., a user operating the device correctly simply by standing on the impedance body scale foot electrodes), and can be used to obtain reliable trigger fiducial timings from the Leg IPG signal. This acquisition can be far less sensitive to motion-induced noise from the Leg EMG that often compromises Leg ECG measurements. Furthermore, it has been discovered that interleaving the two Kelvin electrode pairs for a single foot, result in a design that is insensitive to foot placement within the boundaries of the overall electrode area. As such, the user is not constrained to comply with accurate foot placement on conventional single foot Kelvin arrangements, which are highly prone to introducing motion artifacts into the IPG signal, or result in a loss of contact if the foot is slightly misaligned. Interleaved designs begin when one or more electrode surfaces cross over a single imaginary boundary line separating an excitation and sensing electrode pair. The interleaving is configured to maintain uniform foot surface contact area on the excitation and sensing electrode pair, regardless of the positioning of the foot over the combined area of the electrode pair.

Various aspects of the present disclosure include a weighing scale platform (e.g., scale 110) of an area sufficient for an adult of average size to stand comfortably still and minimize postural swaying. The nominal scale length (same orientation as foot length) is 12 inches and the width is 12 inches. The width can be increased to be consistent with the feet at shoulder width or slightly broader (e.g., 14 to 18 inches, respectively).

FIG. 1e is a flow chart depicting an example manner in which a user-specific physiologic meter or scale may be programmed in accordance with the present disclosure. This flow chart uses a computer processor circuit (or CPU) along with a memory circuit shown herein as user profile memory 146a. The CPU operates in a low-power consumption mode, which may be in off mode or a low-power sleep mode, and at least one other higher power consumption mode of operation. The CPU can be integrated with presence and/or motion sense circuits, such as a passive infrared (PIR) circuit and/or pyroelectric PIR circuit. In a typical application, the PIR circuit provides a constant flow of data indicative of amounts of radiation sensed in a field of view directed by the PIR circuit. For instance, the PIR circuit can be installed behind an upper surface which is transparent to infrared light (and/or other visible light) of the platform and installed at an angle so that the motion of the user approaching the platform apparatus is sensed. Radiation from the user, upon reaching a certain detectable level, wakes up the CPU which then transitions from the low-power mode, as depicted in block 140, to a regular mode of operation. Alternatively, the low-power mode of operation is transitioned from a response to another remote/wireless input used as a presence to awaken the CPU. In other embodiments, user motion can be detected by an accelerometer integrated in the scale or the motion is sensed with a single integrated microphone or microphone array, to detect the sounds of a user approaching.

Accordingly, from block 140, flow proceeds to block 142 where the user or other intrusion is sensed as data received at the platform apparatus. At block 144, the circuitry assesses whether the received data qualifies as requiring a wake up. If not, flow turns to block 140. If however, wake up is required, flow proceeds from block 144 to block 146 where the CPU assesses whether a possible previous user has approached the platform apparatus. This assessment is performed by the CPU accessing the user profile memory 146A and comparing data stored therein for one or more such previous users with criteria corresponding to the received data that caused the wake up. Such criteria includes, for example, the time of the day, the pace at which the user approached the platform apparatus as sensed by the motion detection circuitry, the height of the user as indicated by the motion sensing circuitry and/or a camera installed and integrated with the CPU, and/or more sophisticated bio-metric data provided by the user and/or automatically by the circuitry in the platform apparatus.

As discussed herein, such sophisticated circuitry can include one or more of the following user-specific attributes: foot length, type of foot arch, weight of user, and/or manner and speed at which the user steps onto the platform apparatus or by user speech (e.g., voice). In some embodiments, facial or body-feature recognition may also be used in connection with the camera and comparisons of images therefrom to images in the user profile memory.

From block 146, flow proceeds to block 148 where the CPU obtains and/or updates user corresponding data in the user profile memory. As a learning program is developed in the user profile memory, each access and use of the platform apparatus is used to expand on the data and profile for each such user. From block 148, flow proceeds to block 150 where a decision is made regarding whether the set of electrodes at the upper surface of the platform are ready for the user, such as may be based on the data obtained from the user profile memory. For example, delays may ensue from the user moving his or her feet about the upper surface of the platform apparatus, as may occur while certain data is being retrieved by the CPU (whether internally or from an external source such as a program or configuration data updates from the Internet cloud) or when the user has stepped over the user-display. If the electrodes are not ready for the user, flow proceeds from block 150 to block 152 to accommodate this delay.

Once the CPU determines that the electrodes are ready for use while the user is standing on the platform surface, flow proceeds to block 160. Stabilization of the user on the platform surface may be ascertained by injecting current through the electrodes via the interleaved arrangement thereof. Where such current is returned via other electrodes for a particular foot and/or foot size, and is consistent for a relatively brief period of time, for example, a few seconds, the CPU can assume that the user is standing still and ready to use the electrodes and related circuitry. At block 160, a decision is made that both the user and the platform apparatus are ready for measuring impedance and certain segments of the user's body, including at least one foot.

The remaining flow of FIG. 1e includes the application and sensing of current through the electrodes for finding the optimal electrodes (162) and for performing impedance measurements (block 164). These measurements are continued until completed at block 166 and all such useful measurements are recorded and are logged in the user profile memory for this specific user, at block 168. At block 172, the CPU generates output data to provide feedback as to the completion of the measurements and, as can be indicated as a request via the user profile for this user, as an overall report on the progress for the user and relative to previous measurements made for this user has stored in the user profile memory. Such feedback may be shown on the user-display, through a speaker with co-located apertures in the platform for audible reception by the user, and/or by vibration circuitry which, upon vibration under control of the CPU, the user can sense through one or both feet while standing on the scale. From this output at block 172, flow returns to the low power mode as indicated at block 174 with the return to the beginning of the flow at the block 140.

FIG. 2a shows an example of the insensitivity to foot placement 200 on scale electrode pairs 205/210 with multiple excitation paths 220 and sensing current paths 215, consistent with various aspects of the present disclosure. An aspect of the platform is that it has a thickness and strength to support a human adult of at least 200 pounds without fracturing, and another aspect of the device platform is comprised of at least six electrodes, where the first electrode pair 205 is solid and the second electrode pair 210 are interleaved. Another aspect is the first and second interleaved electrode pairs 205/210 are separated by a distance of at least 40+/−5 millimeters, where the nominal separation of less than 40 millimeters has been shown to degrade the single Foot IPG signal. Another key aspect is the electrode patterns are made from materials with low resistivity such as stainless steel, aluminum, hardened gold, ITO, index matched ITO (IMITO), carbon printed electrodes, conductive tapes, silver-impregnated carbon printed electrodes, conductive adhesives, and similar materials with resistivity lower than 300 ohms/sq. The resistivity can be below 150 ohms/sq. The electrodes are connected to the electronic circuitry in the scale by routing the electrodes around the edges of the scale to the surface below, or through at least one hole in the scale (e.g., a via hole).

Suitable electrode arrangements for dual Foot IPG measurements can be realized in other embodiments. In certain embodiments, the interleaved electrodes are patterned on the reverse side of a thin piece (e.g., less than 2 mm) of high-ion-exchange (HIE) glass, which is attached to a scale substrate and used in capacitive sensing mode. In certain embodiments, the interleaved electrodes are patterned onto a thin piece of paper or plastic which can be rolled up or folded for easy storage. In certain embodiments, the interleaved electrodes are integrated onto the surface of a tablet computer for portable IPG measurements. In certain embodiments, the interleaved electrodes are patterned onto a kapton substrate that is used as a flex circuit.

In certain embodiments, the scale area has a length of 10 inches with a width of eight inches for a miniature scale platform. Alternatively, the scale may be larger (up to 36 inches wide) for use in bariatric class scales.

In the present disclosure, the leg and foot impedance measurements can be simultaneously carried out using a multi-frequency approach, in which the leg and foot impedances are excited by currents modulated at two or more different frequencies, and the resulting voltages are selectively measured using a synchronous demodulator as shown in FIG. 3a. This homodyning approach can be used to separate signals (in this case, the voltage drop due to the imposed current) with very high accuracy and selectivity.

This measurement configuration is based on a four-point configuration in order to minimize the impact of the contact resistance between the electrode and the foot, a practice well-known in the art of impedance measurement. In this configuration the current is injected from a set of two electrodes (the “injection” and “return” electrodes), and the voltage drop resulting from the passage of this current through the resistance is sensed by two separate electrodes (the “sense” electrodes), usually located in the path of the current. Since the sense electrodes are not carrying any current (by virtue of their connection to a high-impedance differential amplifier), the contact impedance does not significantly alter the sensed voltage.

In order to sense two distinct segments of the body (the legs and the foot), two separate current paths are defined by electrode positioning. Therefore two injection electrodes are used, each connected to a current source modulated at a different frequency. The injection electrode for leg impedance is located under the plantar region of the left foot, while the injection electrode for the Foot IPG is located under the heel of the right foot. Both current sources share the same return electrode located under the plantar region of the right foot. This is an illustrative example. Other configurations may be used.

The sensing electrodes can be localized so as to sense the corresponding segments. Leg IPG sensing electrodes are located under the heels of each foot, while the two foot sensing electrodes are located under the heel and plantar areas of the right foot. The inter-digitated nature of the right foot electrodes ensures a four-point contact for proper impedance measurement, irrespectively of the foot position, as already explained.

FIG. 2b shows an example of electrode configurations, consistent with various aspects of the disclosure. As shown by the electrode connections, in some embodiments, ground is coupled to the heel of a first foot of the user (e.g., the right foot) and the foot current injection (e.g., excitation paths 220) is coupled to the toes of the respective first foot (e.g., toes of the right foot). The leg current injection is coupled to the toes of the second foot (e.g., toes of the left foot).

FIG. 2c shows an example of electrode configurations, consistent with various aspects of the disclosure. As shown by the electrode connections, in some embodiments, ground is coupled to the heel of a first foot of the user (e.g., the right foot) and the foot current injection (e.g., excitation paths 220) is coupled to the toes of the first foot (e.g., toes of the right foot). The leg current injection is coupled to the heels of the second foot of the user (e.g., heels of the left foot).

FIGS. 3a-3b show example block diagrams depicting the circuitry for sensing and measuring the cardiovascular time-varying IPG raw signals and steps to obtain a filtered IPG waveform, consistent with various aspects of the present disclosure. The example block diagrams shown in FIGS. 3a-3b are separated in to a leg impedance sub-circuit 300 and a foot impedance sub-circuit 305.

Excitation is provided by way of an excitation waveform circuit 310. The excitation waveform circuit 310 provides a stable amplitude excitation signal by way of various wave shapes of various, frequencies, such as more specifically, a sine wave signal (as is shown in FIG. 3a) or, more specifically, a square wave signal (as shown in FIG. 3b). This excitation waveform (of sine, square, or other wave shape) is fed to a voltage-controlled current source circuit 315 which scales the signal to the desired current amplitude. The generated current is passed through a decoupling capacitor (for safety) to the excitation electrode, and returned to ground through the return electrode (grounded-load configuration). Amplitudes of 1 and 4 mA peak-to-peak are typically used for Leg and Foot IPGs, respectively.

The voltage drop across the segment of interest (legs or foot) is sensed using an instrumentation differential amplifier (e.g., Analog Devices AD8421) 320. The sense electrodes on the scale are AC-coupled to the inputs of the differential amplifier 320 (configured for unity gain), and any residual DC offset is removed with a DC restoration circuit (as exemplified in Burr-Brown App Note Application Bulletin, SBOA003, 1991, or Burr-Brown/Texas Instruments INA118 datasheet). Alternatively, a fully differential input amplification stage can be used which eliminates the need for DC restoration.

The signal is then demodulated with a phase-sensitive synchronous demodulator circuit 325. The demodulation is achieved in this example by multiplying the signal by 1 or −1 synchronously in-phase with the current excitation. Such alternating gain is provided by an operational amplifier (op amp) and an analog switch (SPST), such as an ADG442 from Analog Devices). More specifically, the signal is connected to both positive and negative inputs through 10 kOhm resistors. The output is connected to the negative input with a 10 kOhm resistor as well, and the switch is connected between the ground and the positive input of the op amp. When open, the gain of the stage is unity. When closed (positive input grounded), the stage acts as an inverting amplifier with a gain of −1. Further, fully differential demodulators can alternatively be used which employ pairs of DPST analog switches whose configuration can provide the benefits of balanced signals and cancellation of charge injection artifacts. Alternatively, other demodulators such as analog multipliers or mixers can be used. The in-phase synchronous detection allows the demodulator to be sensitive to only the real, resistive component of the leg or foot impedance, thereby rejecting any imaginary, capacitive components which may arise from parasitic elements associated with the foot to electrode contacts.

Once demodulated, the signal is band-pass filtered (0.4-80 Hz) with a band-pass filter circuit 330 before being amplified with a gain of 100 with a non-inverting amplifier circuit 335 (e.g., using an LT1058 operational amplifier from Linear Technology Inc.). The amplified signal is further amplified by 10 and low-pass filtered (cut-off at 20 Hz) using a low-pass filter circuit 340 such as 2-pole Sallen-Key filter stage with gain. The signal is then ready for digitization and further processing. In certain embodiments, the signal from the demodulator circuit 325 can be passed through an additional low-pass filter circuit 345 to determine body or foot impedance.

In certain embodiments, the generation of the excitation voltage signal, of appropriate frequency and amplitude, is carried out by a microcontroller, such as an MSP430 (Texas Instruments, Inc.) or a PIC18Fxx series (Microchip Technology, Inc.). The voltage waveform can be generated using the on-chip timers and digital input/outputs or pulse width modulation (PWM) peripherals, and scaled down to the appropriate voltage through fixed resistive dividers, active attenuators/amplifiers using on-chip or off-chip operational amplifiers, as well as programmable gain amplifiers or programmable resistors. In certain embodiments, the generation of the excitation frequency signal can be accomplished by an independent quartz crystal oscillator whose output is frequency divided down by a series of toggle flip-flops (such as an ECS-100AC from ECS International, Inc., and a CD4024 from Texas Instruments, Inc.). In certain embodiments, the generation of the wave shape and frequency can be accomplished by a direct digital synthesis (DDS) integrated circuit (such as an AD9838 from Analog Devices, Inc.). In certain embodiments, the generation of the wave shape (either sine or square) and frequency can be accomplished by a voltage-controlled oscillator (VCO) which is controlled by a digital microcontroller, or which is part of a phase-locked loop (PLL) frequency control circuit. Alternatively, the waveforms and frequencies can be directly generated by on- or off-chip digital-to-analog converters (DACs).

In certain embodiments, the shape of the excitation is not square, but sinusoidal. Such configuration can reduce the requirements on bandwidth and slew rate for the current source and instrumentation amplifier. Harmonics, potentially leading to higher electromagnetic interference (EMI), can also be reduced. Such excitation may also reduce electronics noise on the circuit itself. Lastly, the lack of harmonics from sine wave excitation may provide a more flexible selection of frequencies in a multi-frequency impedance system, as excitation waveforms have fewer opportunities to interfere between each other. Due to the concentration of energy in the fundamental frequency, sine wave excitation could also be more power-efficient. In certain embodiments, the shape of the excitation is not square, but trapezoidal. Alternatively, raised cosine pulses (RCPs) could be used as the excitation wave shape, providing an intermediate between sine and square waves. RCPs could provide higher excitation energy content for a given amplitude, but with greatly reduced higher harmonics.

To further reduce potential electromagnetic interference (EMI), other strategies may be used, such as by dithering the square wave signal (i.e., introducing jitter in the edges following a fixed or random pattern) which leads to so-called spread spectrum signals, in which the energy is not localized at one specific frequency (or a set of harmonics), but rather distributed around a frequency (or a set of harmonics). Because of the synchronous demodulation scheme, phase-to-phase variability introduced by spread-spectrum techniques will not affect the impedance measurement. Such a spread-spectrum signal can be generated by, but not limited to, specialized circuits (e.g., Maxim MAX31C80, SiTime SiT9001), or generic microcontrollers (see Application Report SLAA291, Texas Instruments, Inc.). These spread-spectrum techniques can be combined with clock dividers to generate lower frequencies as well.

As may be clear to one skilled in the art, these methods of simultaneous measurement of impedance in the leg and foot can be used for standard Body Impedance Analysis (BIA), aiming at extracting the relative content of total water, free-water, fat mass and other body composition measures. Impedance measurements for BIA are typically done at frequencies ranging from kilohertz up to several megahertz. The multi-frequency synchronous detection measurement methods described above can readily be used for such BIA, provided that low-pass filtering (345, FIGS. 3a and 3b) instead of band-pass filtering (330, FIGS. 3a and 3b) is performed following the demodulation. In certain embodiments, a separate demodulator channel may be driven by the quadrature phase of the excitation signal to allow the imaginary component of the body impedance to be extracted in addition to the real component. A more accurate BIA can be achieved by measuring both the real and imaginary components of the impedance. This multi-frequency technique can be combined with traditional sequential measurements used for BIA, in which the impedance is measured at several frequencies sequentially. These measurements are repeated in several body segments for segmental BIAs, using a switch matrix to drive the current into the desired body segments.

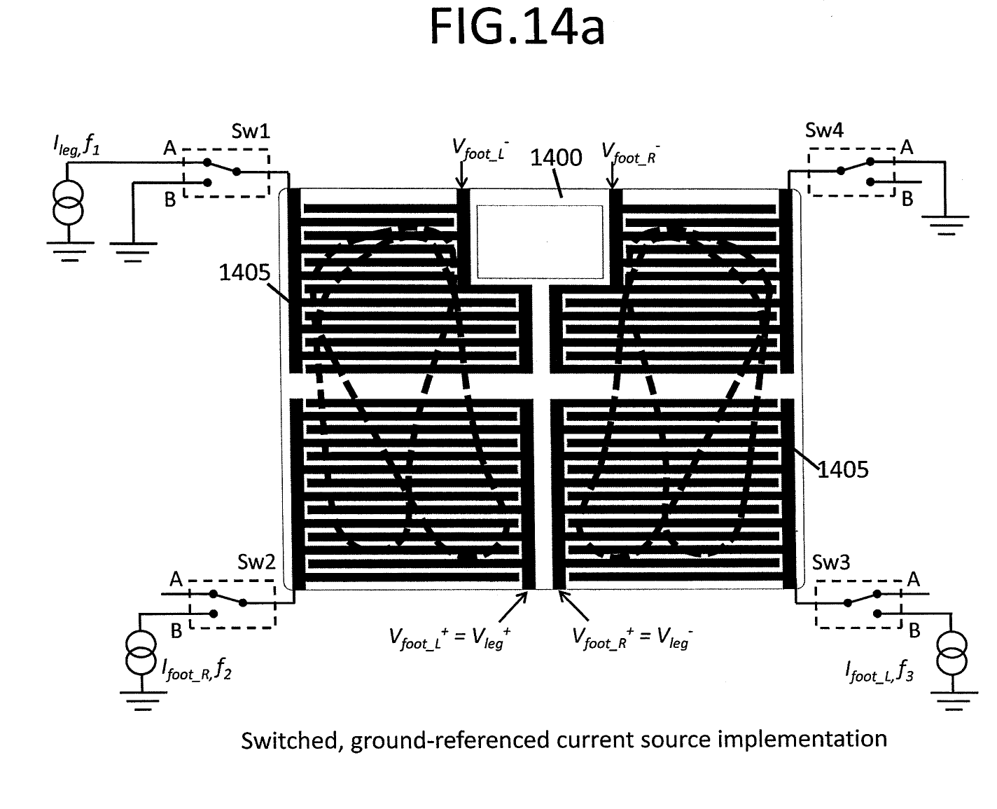

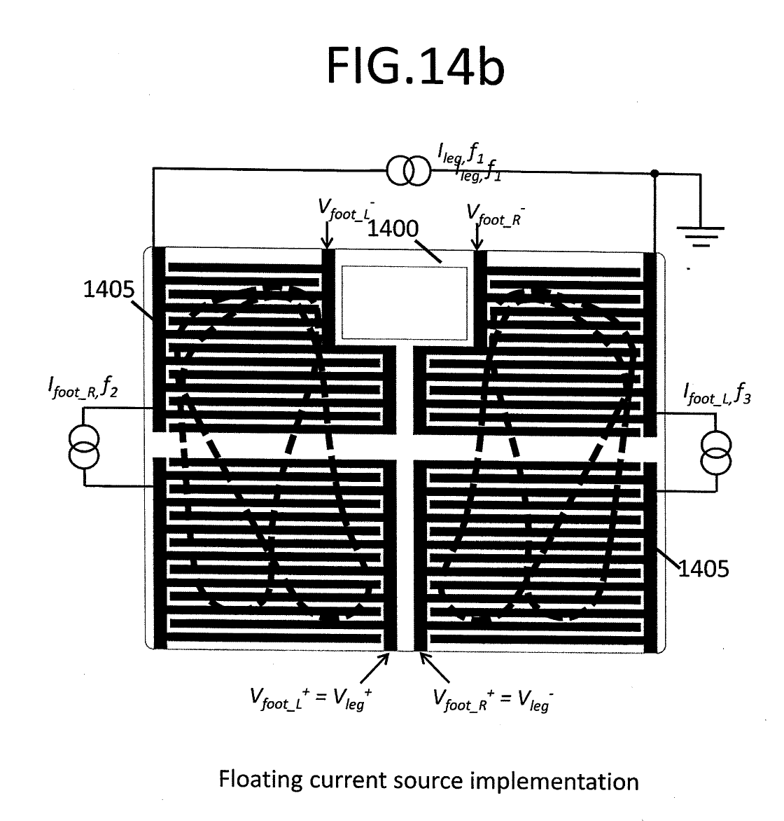

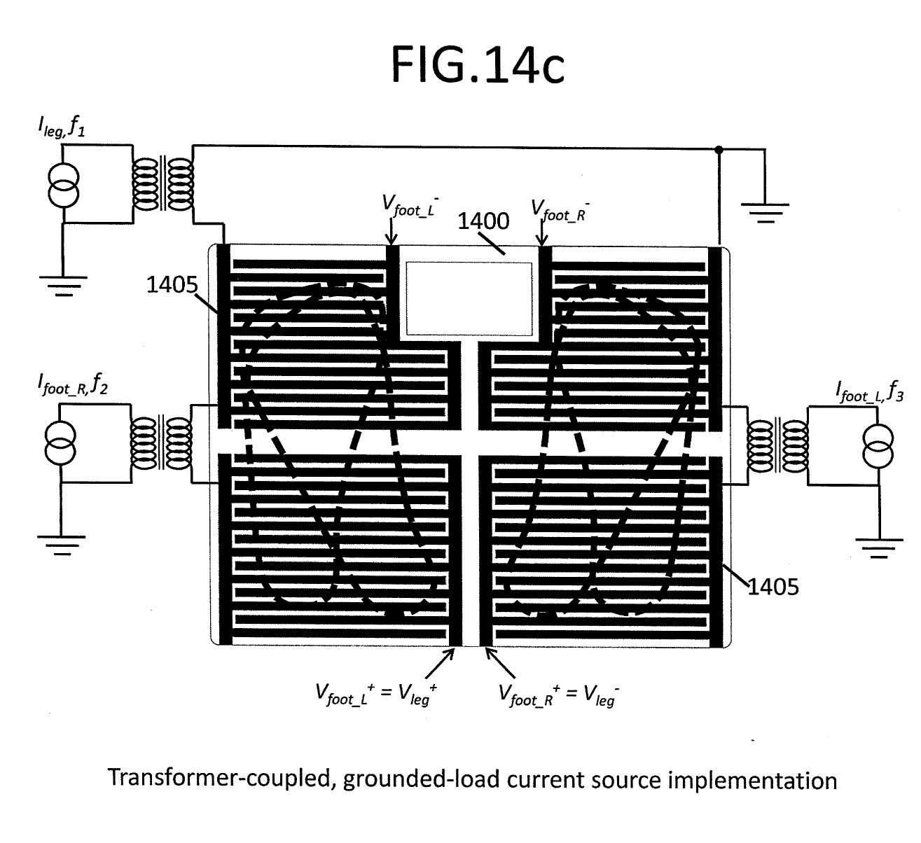

While FIG. 2a shows a circuit and electrode configuration suitable to measure two different segments (legs and one foot), this approach is not readily extendable to more segments due to the shared current return electrode (ground). To overcome this limitation, and provide simultaneous measurements in both feet, the system can be augmented with analog switches to provide time-multiplexing of the impedance measurements in the different segments. This multiplexing can be a one-time sequencing (each segment is measured once), or interleaved at a high-enough frequency that the signal can be simultaneously measured on each segment. The minimum multiplexing rate for proper reconstruction is twice the bandwidth of the measured signal, based on signal processing theory (the Nyquist rate), which equals to about 100 Hz for the impedance signal considered here. The rate must also allow for the signal path to settle in between switching, which usually limits the maximum multiplexing rate. Referring to FIG. 14a, one cycle might start the measurement of the leg impedance and left foot impedances (similarly to previously described, sharing a common return electrode), but then follow with a measurement of the right foot after reconfiguring the switches. For specific information regarding typical switch configurations, reference to U.S. patent application Ser. No. 14/338,266 filed on Oct. 7, 2015, which is fully incorporated for its specific and general teaching of switch configurations.

Since right and left feet are measured sequentially, one should note that a unique current source (at the same frequency) may be used to measure both, providing that the current source is not connected to the two feet simultaneously through the switches, in which case the current would be divided between two paths. One should also note that a fully-sequential measurement, using a single current source (at a single frequency) successively connected to the three different injection electrodes, could be used as well, with the proper switch configuration sequence (no splitting of the current path).

In certain embodiments, the measurement of various body segments, and in particular the legs, right foot and left foot, is achieved simultaneously due to as many floating current sources as segments to be measured, running at separate frequency so they can individually be demodulated. Such configuration is exemplified in FIG. 14b for three segments (legs, right and left feet). Such configuration has the advantage to provide true simultaneous measurements without the added complexity of time-multiplexing/demultiplexing, and associated switching circuitry. An example of such a floating current source is found in Plickett, et al., Physiological Measurement, 32 (2011). Another approach to floating current sources is the use of transformer-coupled current sources (as depicted in FIG. 14c). Using transformers to inject current into the electrodes enables the use of simpler, grounded-load current sources on the primary, while the electrodes are connected to the secondary. The transformer turns ratio can typically be 1:1, and since frequencies of interest for impedance measurement are typically in the 10-1000 kHz (occasionally 1 kHz for BIA), relatively small pulse transformers can be used. In order to limit the common mode voltage of the body, one of the electrodes in contact with the foot can be grounded.

While certain embodiments presented in the above specification have used current sources for excitation, the excitation can also be performed by a voltage source, where the resulting injection current is monitored by a current sense circuit so that impedance can still be derived by the ratio of the sensed voltage (on the sense electrodes) over the sensed current (injected in the excitation electrodes). It should be noted that broadband spectroscopy methods could also be used for measuring impedances at several frequencies. Combined with time-multiplexing and current switching described above, multi-segment broadband spectroscopy can be achieved.

Various aspects of the present disclosure are directed toward robust timing extraction of the blood pressure pulse in the foot which is achieved by means of a two-step processing. In a first step, the usually high-SNR Leg IPG is used to derive a reference (trigger) timing for each heart pulse. In a second step, a specific timing in the lower-SNR Foot IPG is extracted by detecting its associated feature within a restricted window of time around the timing of the Leg IPG.

Consistent with yet further embodiments of the present disclosure, FIG. 3c depicts an example block diagram of circuitry for operating core circuits and modules, including, for example, the operation of the CPU as in FIG. 1a with the related more specific circuit blocks/modules in FIGS. 3A-3B. As shown in the center of FIG. 3c, the computer circuit 370 is shown with other previously-mentioned circuitry in a generalized manner without showing some of the detailed circuitry (e.g., amplification and current injection/sensing (372)). The computer circuit 370 can be used as a control circuit with an internal memory circuit (or as integrated with the memory circuit for the user profile memory 146A of FIG. 1a) for causing, processing and/or receiving sensed input signals as at block 372. As discussed, these sensed signals can be responsive to injection current and/or these signals can be sensed by less complex grid-based sense circuitry surrounding the platform as is convention in capacitive touch-screen surfaces which, in certain embodiments, the platform includes.

As noted, the memory circuit can be used not only for the user profile memory, but also as to provide configuration and/or program code and/or other data such as user-specific data from another authorized source such as from a user monitoring his/her logged data and/or profile from a remote desk-top. The remote device or desk-top can communicate with and access such data via a wireless communication circuit 376. For example, the wireless communication circuit 376 provides an interface between an app on the user's cellular telephone/tablet and the apparatus, wherefrom the IPhone is the output/input interface for the platform (scale) apparatus including, for example, an output display, speaker and/or microphone, and vibration circuitry; each of these I/O aspects and components being discussed herein in connection with other example embodiments.

A camera 378 and image encoder circuit 380 (with compression and related features) can also be incorporated as an option. As discussed above, the weighing scale components, as in block 382, are also optionally included in the housing which encloses and/or surrounds the platform.

For long-lasting battery life in the platform apparatus (batteries not shown), at least the CPU 370, the wireless communication circuit 376, and other current draining circuits are inactive unless and until activated in response to the intrusion/sense circuitry 388. As shown, one specific implementation employs a Conexant chip (e.g., CX93510) to assist in the low-power operation. This type of circuitry is designed for motion sensors configured with a camera for visual verification and image and video monitoring applications (such as by supporting JPEG and MJPEG image compression and processing for both color and black and white images). When combined with an external CMOS sensor, the chip retrieves and stores compressed JPEG and audio data in an on-chip memory circuit (e.g., 256 KB/128 KB frame buffer) to alleviate the necessity of external memory. The chip uses a simple register set via the microprocessor interface and allows for wide flexibility in terms of compatible operation with another microprocessor.

In one specific embodiment, a method of using the platform with the plurality of electrodes are concurrently contacting a limb of the user, includes operating such to automatically obtain measurement signals from the plurality of electrodes. As noted above, these measurement signals might initially be through less complex (e.g., capacitive grid-type) sense circuitry. Before or while obtaining a plurality of measurement signals by operating the circuitry, the signal-sense circuitry 388 is used to sense wireless-signals indicative of the user approaching the platform and, in response, causing the CPU circuitry 370 to transition from a reduced power-consumption mode of operation and at least one higher power-consumption mode of operation. After the circuitry is operating in the higher power-consumption mode of operation, the CPU accesses the user-corresponding data stored in the memory circuit and causes a plurality of impedance-measurement signals to be obtained by using the plurality of electrodes while they are contacting the user via the platform; therefrom, the CPU generates signals corresponding to cardiovascular timings of the user.

The signal-sense circuit can be employed as a passive infrared detector and with the CPU programmed (as a separate module) to evaluate whether radiation from the passive infrared detector is indicative of a human. For example, sensed levels of radiation that corresponds to a live being, such as a dog, that is less than a three-foot height, and/or has not moved for more than a couple seconds, can be assessed as being a non-human.

Accordingly, as the user is recognized as being human, the CPU is activated and begins to attempt the discernment process of which user might be approaching. This is performed by the CPU accessing the user-corresponding data stored in the memory circuit (the user profile memory). If the user is recognized based on parameters such as discussed above (e.g., time of morning, speed of approach, etc.), the CPU can also select one of a plurality of different types of user-discernible visual/audible/tactile information and for presenting the discerned user with visual/audible/tactile information that was retrieved from the memory as being specific to the user. For example, user-selected visual/audible data can be outputted for the user. Also, responsive to the motion detection indication, the camera can be activated to capture at least one image of the user while the user is approaching the platform (and/or while the user is on the platform to log confirmation of the same user with the measured impedance information). As shown in block 374 of FIG. 3c, where a speaker is also integrated with the CPU, the user can simply command the platform apparatus to start the process and activation proceeds. As previously discussed, the scale can include voice input/output circuitry to receive the user commands via voice commands.

In another method, the circuitry of FIG. 3c is used with the electrodes being interleaved and engaging the user, as a combination weighing scale (via block 382) and a physiologic user-specific impedance-measurement device. By using the impedance-measurement signals and obtaining at least two impedance-measurement signals between one foot of the user and another location of the user, the interleaved electrodes assist the CPU in providing measurement results that indicate one or more of the following user-specific attributes as being indicative or common to the user: foot impedance, foot length, and type of arch, and wherein one or more of the user-specific attributes are accessed in the memory circuit and identified as being specific to the user. This information can be later retrieved by the user, medical and/or security personnel, according to a data-access authorization protocol as might be established upon initial configuration for the user.

FIG. 3d shows an exemplary block diagram depicting the circuitry for interpreting signals received from electrodes (e.g., 372 of FIG. 3c), and/or CPU 370 of FIG. 3c. The input electrodes 375 transmit electrical signals through the patient's body (depending on the desired biometric and physiological test to be conducted) and output electrodes 380 receive the modified signal as affected by a user's electrical impedance 385. Once received by the output electrodes 380, the modified signal is processed by processor circuitry 370 based on the selected test. Signal processing conducted by the processor circuitry 370 is discussed in more detail above (with regard to FIGS. 3a-b). In certain embodiments of the present disclosure, the circuitry within 370 is provided by Texas Instruments part # AFE4300.

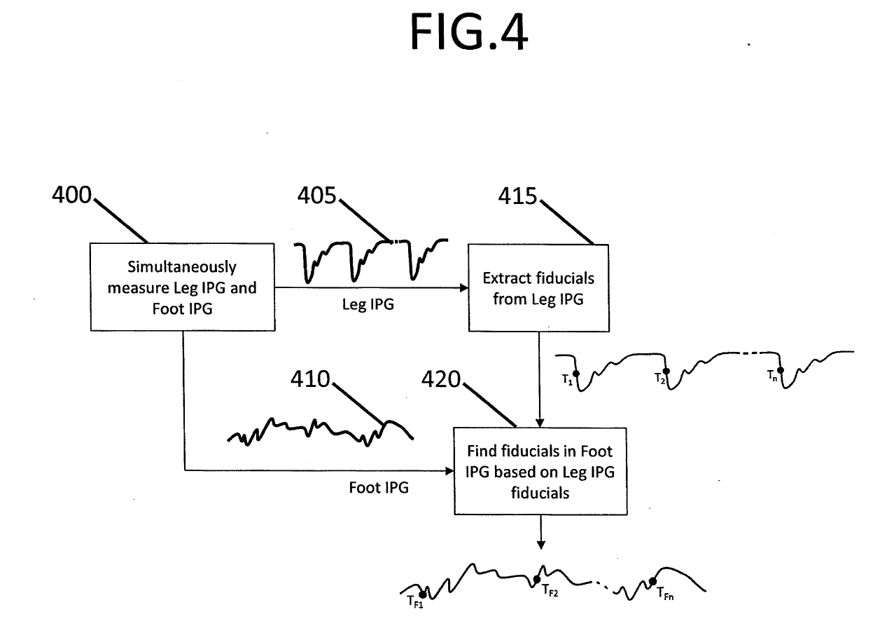

FIG. 4 shows an example block diagram depicting signal processing steps to obtain fiducial references from the individual Leg IPG “beats,” which are subsequently used to obtain fiducials in the Foot IPG, consistent with various aspects of the present disclosure. In the first step, as shown in block 400, the Leg IP and the Foot IPG are simultaneously measured. As shown at 405, the Leg IPG is low-pass filtered at 20 Hz with an 8-pole Butterworth filter, and inverted so that pulses have an upward peak. The location of the pulses is then determined by taking the derivative of this signal, integrating over a 100 ms moving window, zeroing the negative values, removing the large artifacts by zeroing values beyond 15× the median of the signal, zeroing the values below a threshold defined by the mean of the signal, and then searching for local maxima. Local maxima closer than a defined refractory period of 300 ms to the preceding ones are dismissed. The result is a time series of pulse reference timings.

As is shown in 410, the foot IPG is low-pass filtered at 25 Hz with an 8-pole Butterworth filter and inverted (so that pulses have an upward peak). Segments starting from the timings extracted (415) from the Leg IPG (reference timings) and extending to 80% of the previous pulse interval, but no longer than one second, are defined in the Foot IPG. This defines the time windows where the Foot IPG is expected to occur, avoiding misdetection outside of these windows. In each segment, the derivative of the signal is computed, and the point of maximum positive derivative (maximum acceleration) is extracted. The foot of the IPG signal is then computed using an intersecting tangent method, where the fiducial (420) is defined by the intersection between a first tangent to the IPG at the point of maximum positive derivative and a second tangent to the minimum of the IPG on the left of the maximum positive derivative within the segment.

The time series resulting from this two-step extraction is used with another signal to facilitate further processing. These timings are used as reference timings to improve the SNR of BCG signals to extract intervals between a timing of the BCG (typically the I-wave) and the Foot IPG for the purpose of computing the PWV, as previously disclosed in U.S. 2013/0310700 (Wiard). In certain embodiments, the timings of the Leg IPG are used as reference timings to improve the SNR of BCG signals, and the foot IPG timings are used to extract intervals between timing fiducials of the improved BCG (typically the I-wave) and the Foot IPG for the purpose of computing the PTT and the (PWV).

In certain embodiments, the processing steps include an individual pulse SNR computation after individual timings are extracted, either in Leg IPG or Foot IPG. Following the computation of the SNRs, pulses with a SNR below a threshold value are eliminated from the time series, to prevent propagating noise. The individual SNRs may be computed in a variety of methods known to one skilled in the art. For instance, an estimated pulse can be computed by ensemble averaging segments of signal around the pulse reference timing. The noise associated with each pulse is defined as the difference between the pulse and the estimated pulse. The SNR is the ratio of the root-mean-square (RMS) value of the estimated pulse over the RMS value of the noise for that pulse.

In certain embodiments, the time interval between the Leg IPG pulses, and the Foot IPG pulses, also detected by the above-mentioned methods, is extracted. The Leg IPG measuring a pulse occurring earlier in the legs compared to the pulse from the Foot IPG, the interval between these two is related to the propagation speed in the lower body, i.e., the peripheral vasculature. This provides complementary information to the interval extracted between the BCG and the Foot IPG for instance, and is used to decouple central versus peripheral vascular properties. It is also complementary to information derived from timings between the BCG and the Leg ICG.

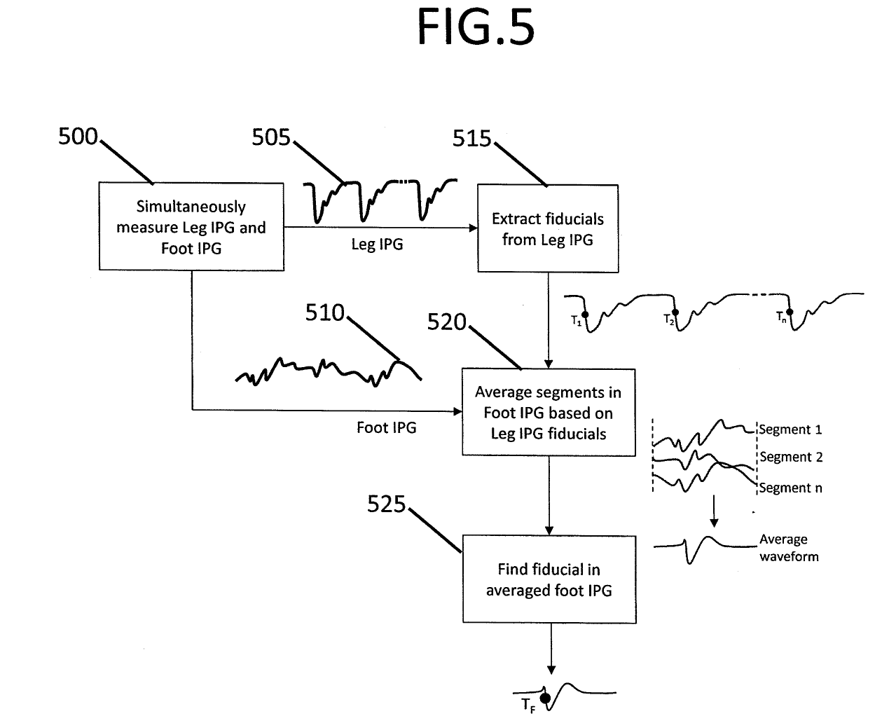

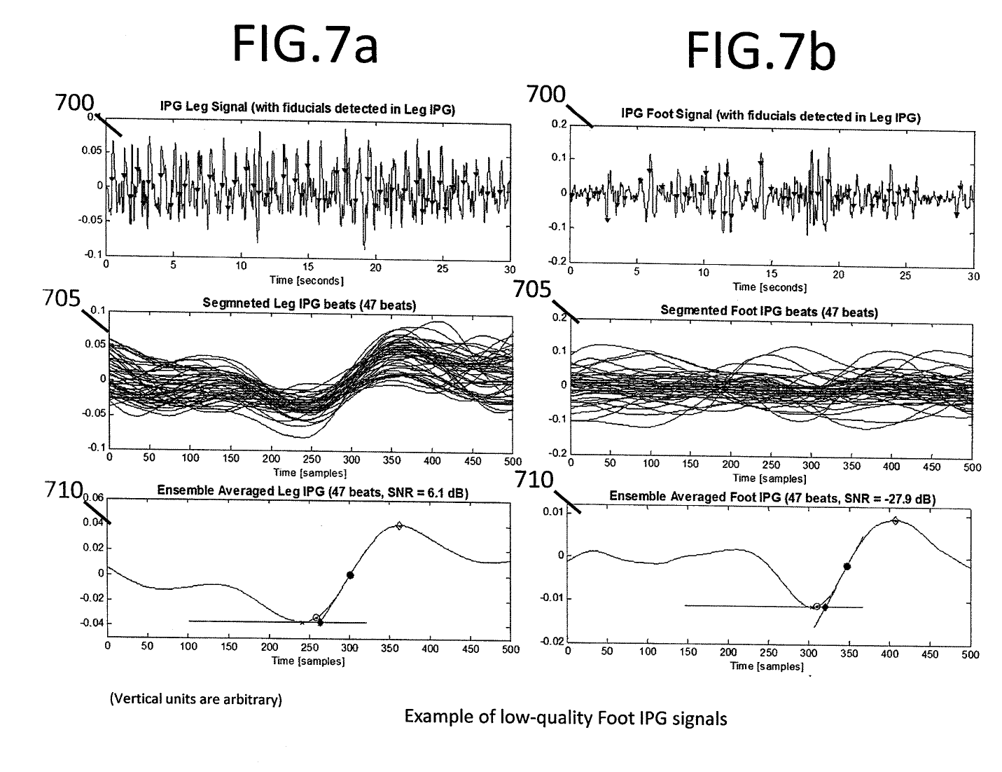

FIG. 5 shows an example flowchart depicting signal processing to segment individual Foot IPG “beats” to produce an averaged IPG waveform of improved SNR, which is subsequently used to determine the fiducial of the averaged Foot IPG, consistent with various aspects of the present disclosure. Similar to the method shown in FIG. 4, the Leg IP and the Foot IPG are simultaneously measured (500), the Leg IPG is low-pass filtered (505), the foot IPG is low-pass filtered (510), and segments starting from the timings extracted (515) from the Leg IPG (reference timings). The segments of the Foot IPG extracted based on the Leg IPG timings are ensemble-averaged (520) to produce a higher SNR Foot IPG pulse. From this ensemble-averaged signal, the start of the pulse is extracted using the same intersecting tangent approach as described earlier. This approach enables the extraction of accurate timings in the Foot IPG even if the impedance signal is dominated by noise, as shown in FIG. 7b. These timings are used together with timings extracted from the BCG for the purpose of computing the PTT and (PWV). Timings derived from ensemble-averaged waveforms and individual waveforms can also be both extracted, for the purpose of comparison, averaging and error-detection.

Specific timings extracted from the IPG pulses (from either leg or foot) are related (but not limited) to the peak of the pulse, the minimum preceding the peak, or the maximum second derivative (maximum rate of acceleration) preceding the point of maximum derivative. An IPG pulse and the extraction of a fiducial (525) in the IPG can be performed by other signal processing methods, including (but not limited to) template matching, cross-correlation, wavelet-decomposition, or short window Fourier transform.

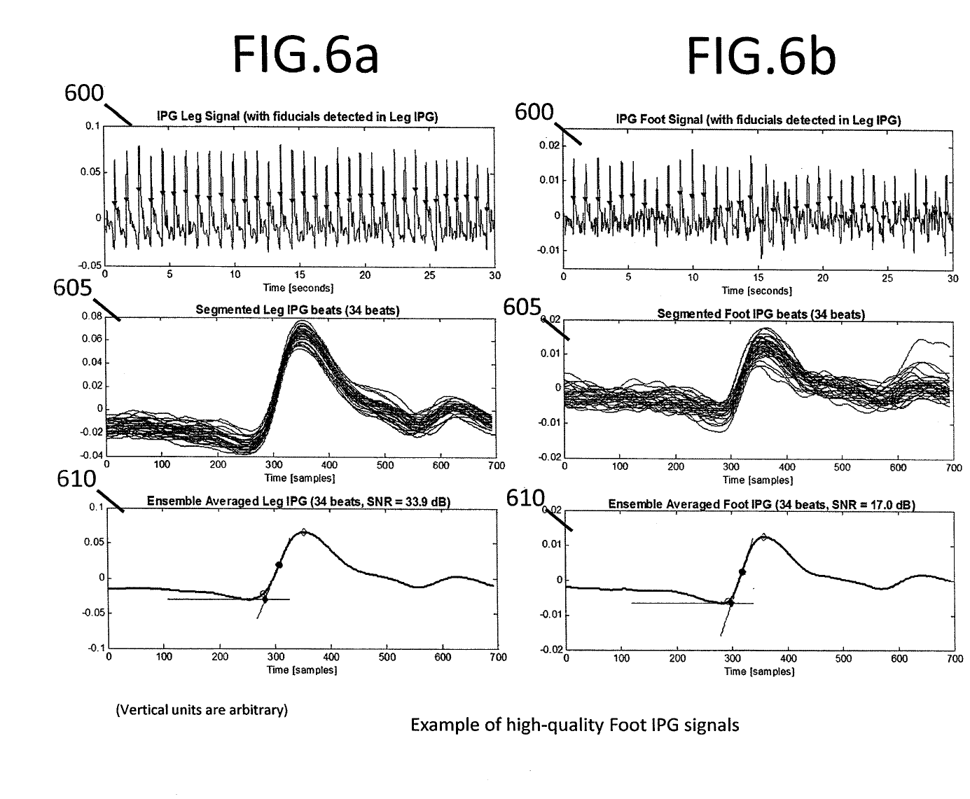

FIG. 6a shows examples of the Leg IPG signal with fiducials (plot 600); the segmented Leg IPG into beats (plot 605); and the ensemble-averaged Leg IPG beat with fiducials and calculated SNR (plot 610), for an exemplary high-quality recording, consistent with various aspects of the present disclosure. FIG. 6b shows examples of the Foot IPG signal with fiducials derived from the Leg IPG fiducials (plot 600); the segmented Foot IPG into beats (plot 605); and the ensemble-averaged Foot IPG beat with fiducials and calculated SNR (plot 610), for an exemplary high-quality recording, consistent with various aspects of the present disclosure.

FIG. 7a shows examples of the Leg IPG signal with fiducials (plot 700); the segmented Leg IPG into beats (plot 705); and the ensemble averaged Leg IPG beat with fiducials and calculated SNR (plot 710), for an exemplary low-quality recording, consistent with various aspects of the present disclosure.

FIG. 7b shows examples of the Foot IPG signal with fiducials derived from the Leg IPG fiducials (plot 700); the segmented Foot IPG into beats (plot 705); and the ensemble-averaged Foot IPG beat with fiducials and calculated SNR (plot 710), for an exemplary low-quality recording, consistent with aspects of the present disclosure.

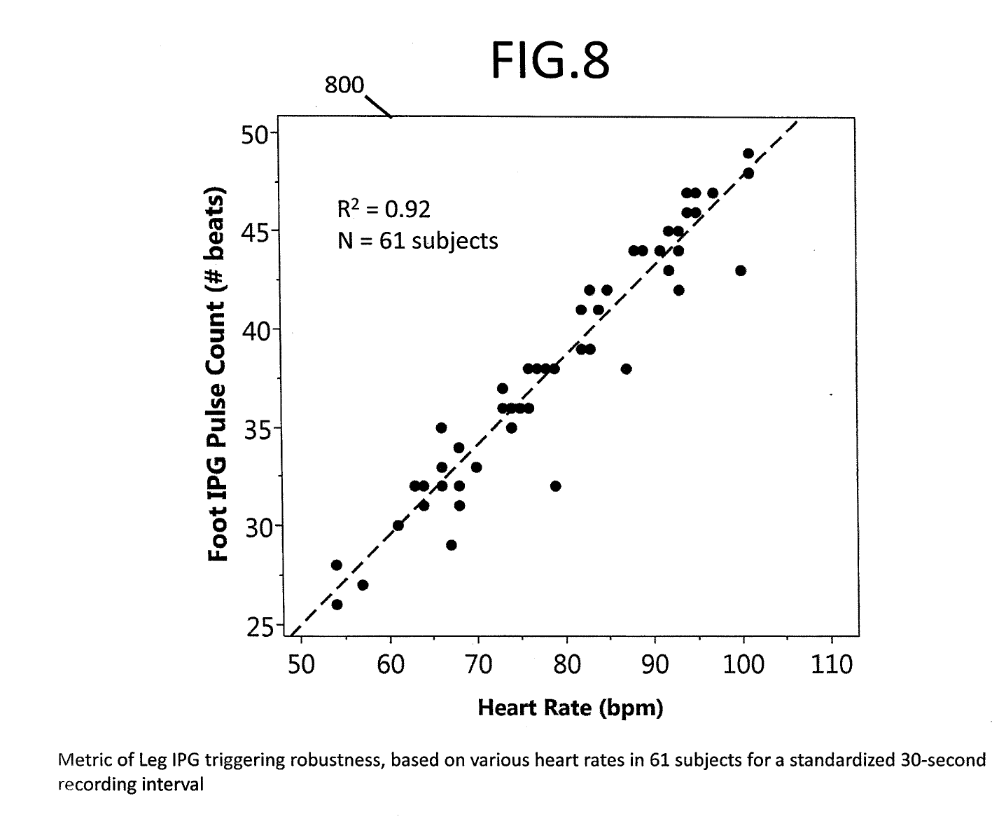

FIG. 8 shows an example correlation plot 800 for the reliability in obtaining the low SNR Foot IPG pulse for a 30-second recording, using the first impedance signal as the trigger pulse, from a study including 61 test subjects with various heart rates, consistent with various aspects of the present disclosure.

In certain embodiments, a dual-Foot IPG is measured, allowing the detection of blood pressure pulses in both feet. Such information can be used for diagnostic of peripheral arterial diseases (PAD) by comparing the relative PATs in both feet to look for asymmetries. It can also increase the robustness of the measurement by allowing one foot to have poor contact with electrodes (or no contact at all). SNR measurements can be used to assess the quality of the signal in each foot, and to select the best one for downstream analysis. Timings extracted from each foot can be compared and set to flag potentially inaccurate PWV measurements due to arterial peripheral disease, in the event these timings are different by more than a threshold. Alternatively, timings from both feet are pooled to increase the overall SNR if their difference is below the threshold.

In certain embodiments, the disclosure is used to measure a PWV, where the IPG is augmented by the addition of BCG sensing into the weighing scale to determine characteristic fiducials between the BCG and Leg IPG trigger, or the BCG and Foot IPG. The BCG sensors are comprised typically of the same strain gage set used to determine the bodyweight of the user. The load cells are typically wired into a bridge configuration to create a sensitive resistance change with small displacements due to the ejection of the blood into the aorta, where the circulatory or cardiovascular force produce movements within the body on the nominal order of 1-3 Newtons. BCG forces can be greater than or less than the nominal range in cases such as high or low cardiac output.

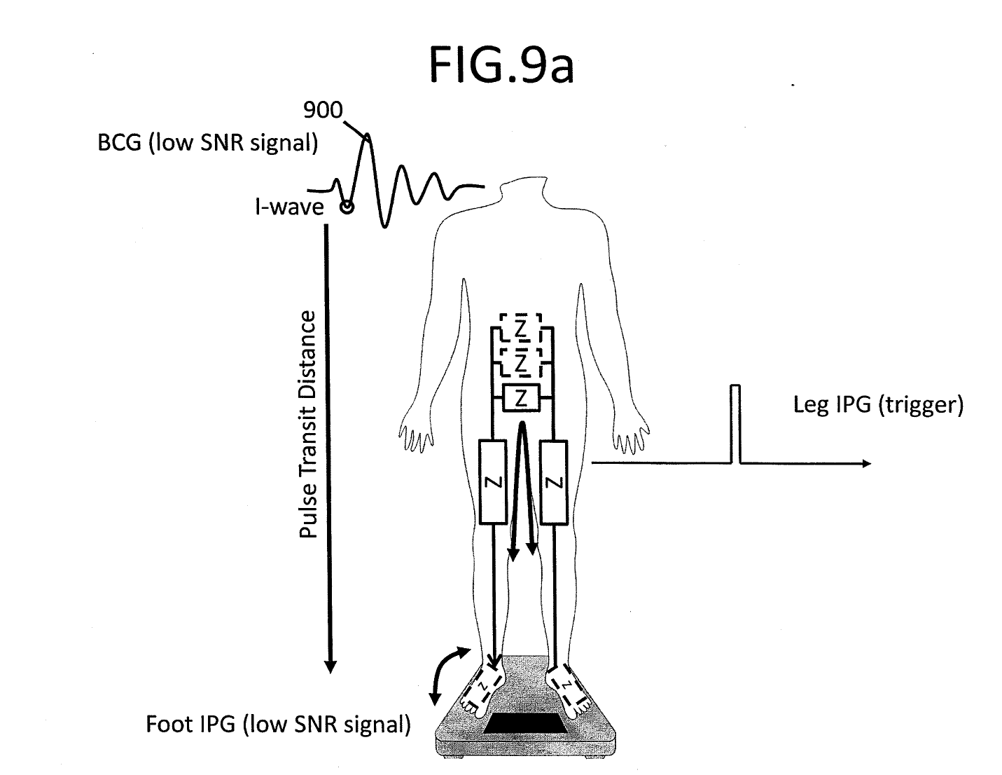

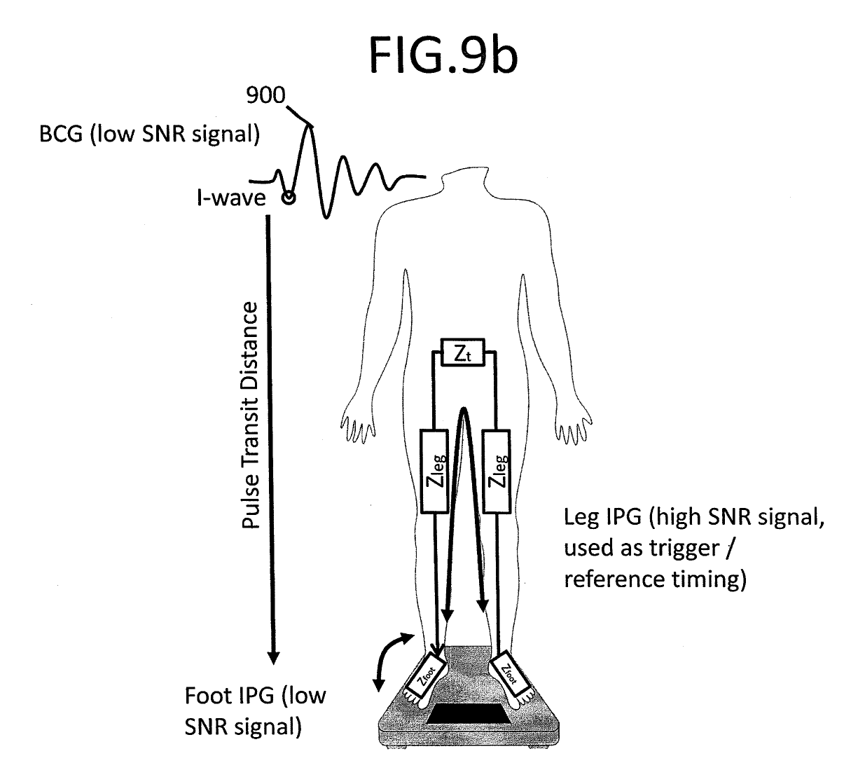

FIGS. 9a-b show example configurations to obtain the PTT, using the first IPG as the triggering pulse for the Foot IPG and BCG, consistent with various aspects of the present disclosure. The I-wave of the BCG 900 normally depicts the headward force due to cardiac ejection of blood into the ascending aorta which is used as a timing fiducial indicative of the pressure pulse initiation of the user's proximal aorta relative to the user's heart. The J-wave is indicative of timings in the systole phase and also incorporates information related to the strength of cardiac ejection and the ejection duration. The K-Wave provides systolic and vascular information of the user's aorta. The characteristic timings of these and other BCG waves are used as fiducials that can be related to fiducials of the IPG signals of the present disclosure.

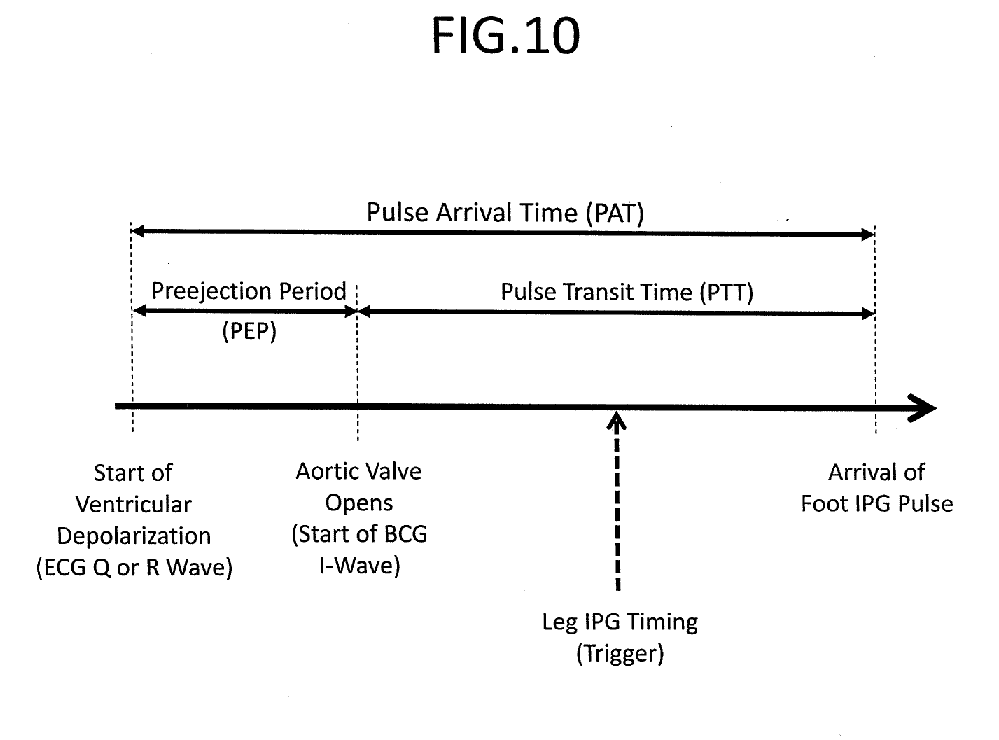

FIG. 10 shows nomenclature and relationships of various cardiovascular timings, consistent with various aspects of the present disclosure.

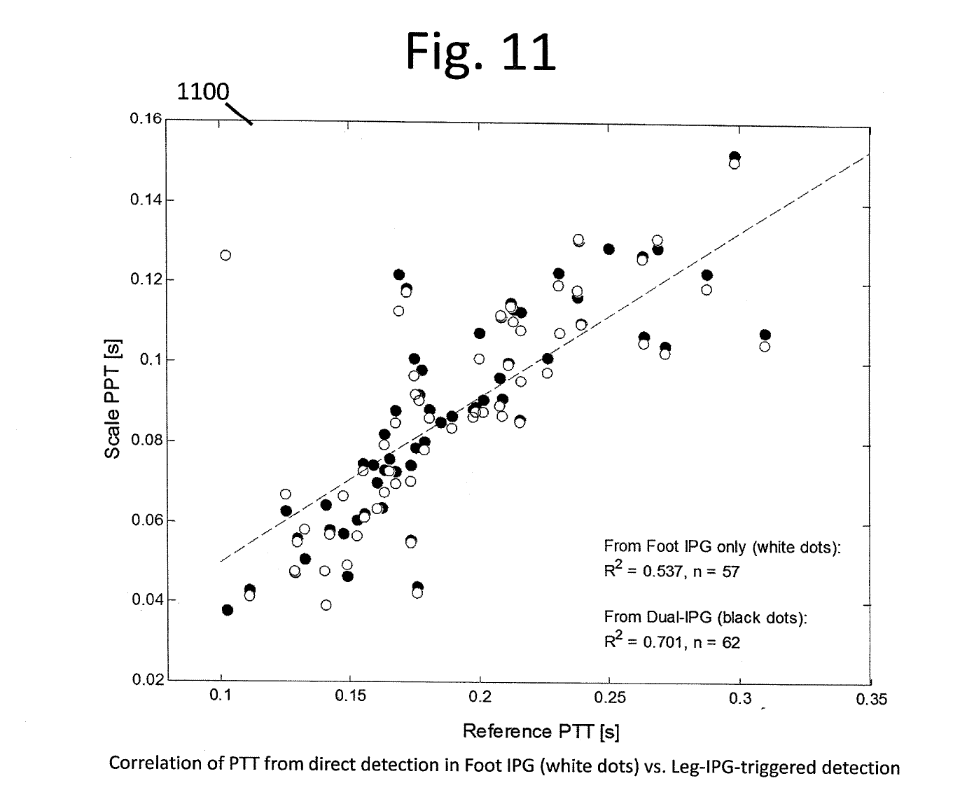

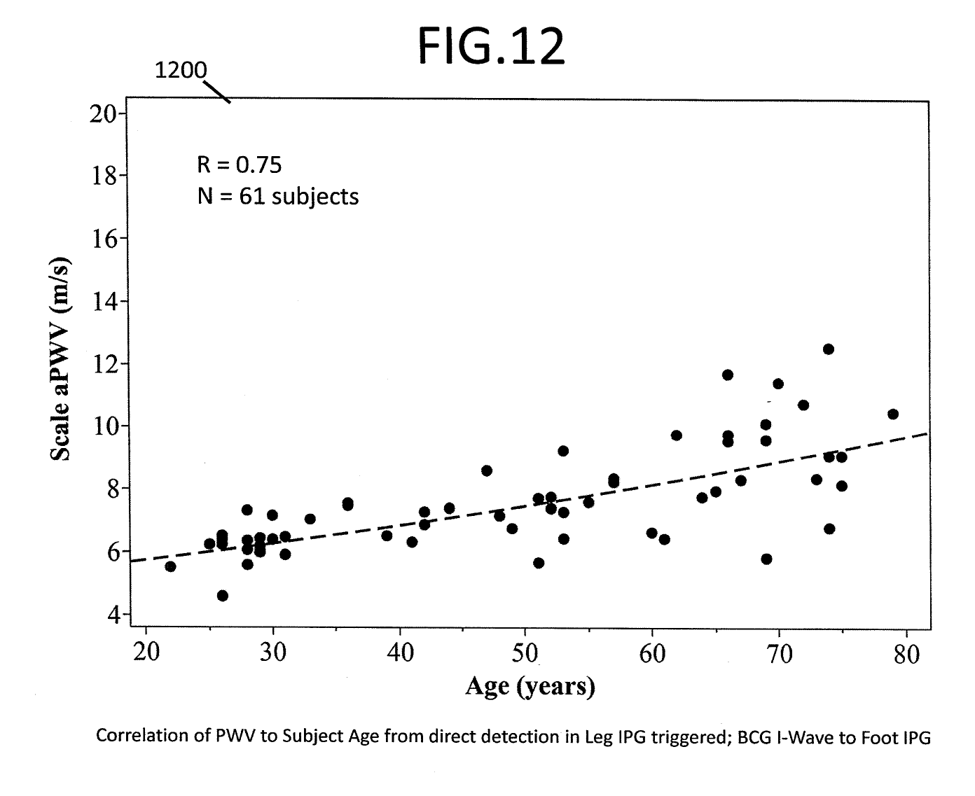

FIG. 11 shows an example graph 1100 of PTT correlations for two detection methods (white dots) Foot IPG only, and (black dots) Dual-IPG method; and FIG. 12 shows an example graph 1200 of PWV obtained from the present disclosure compared to the ages of 61 human test subjects, consistent with various aspects of the present disclosure.

FIG. 13 shows an example of a scale 1300 with integrated foot electrodes 1305 to inject and sense current from one foot to another foot, and within one foot.

FIG. 14a-c shows various examples of a scale 1400 with interleaved foot electrodes 1405 to inject/sense current from one foot to another foot, and measure Foot IPG signals in both feet.

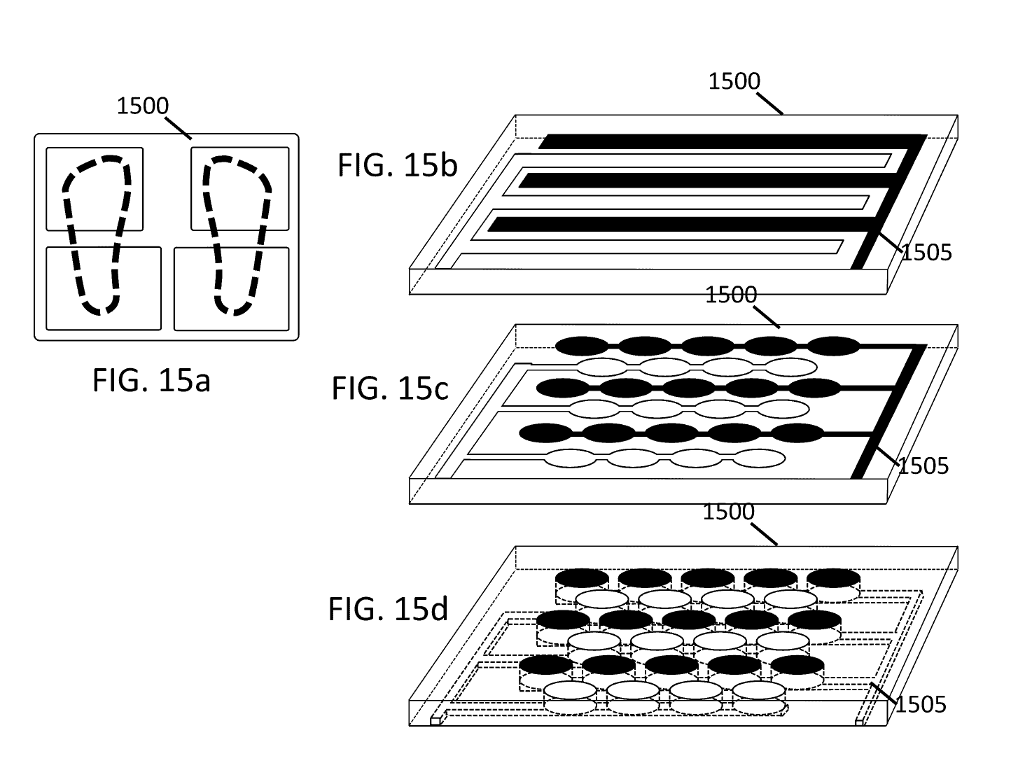

FIGS. 15a-d shows an example breakdown of a scale 1500 with interleaved foot electrodes 1505 to inject and sense current from one foot to another foot, and within one foot.

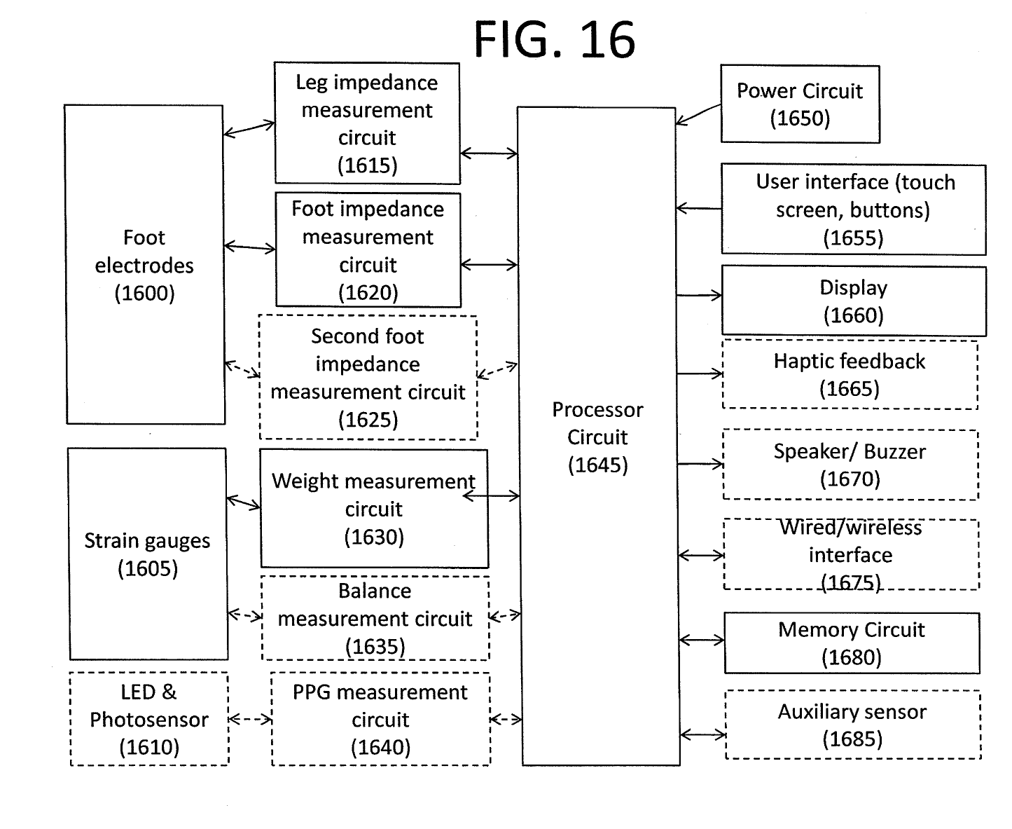

FIG. 16 shows an example block diagram of circuit-based building blocks, consistent with various aspects of the present disclosure. The various circuit-based building blocks shown in FIG. 16 can be implemented in connection with the various aspects discussed herein. In the example shown, the block diagram includes foot electrodes 1600 that can collect the IPG signals. Further, the block diagram includes strain gauges 1605, and an LED/photosensor 1610. The foot electrodes 1600 is configured with a leg impedance measurement circuit 1615, a foot impedance measurement circuit 1620, and an optional second foot impedance measurement circuit 1625. The leg impedance measurement circuit 1615, the foot impedance measurement circuit 1620, and the optional second foot impedance measurement circuit 1625 report the measurements collected to a processor circuitry 1645.

The processor circuitry 1645 collects data from a weight measurement circuit 1630 and an optional balance measurement circuit 1635 that are configured with the strain gauges 1605. Further, an optional photoplethysmogram (PPG) measurement circuit 1640, which collects data from the LED/photosensor 1610, provides data to the processor circuitry 1645.

The processor circuitry 1645 is powered via a power circuit 1650. Further, the processor circuitry 1645 collects user input data from a user interface 1655 (e.g., iPad®, smart phone and/or other remote user handy/CPU with a touch screen and/or buttons). The data collected/measured by the processor circuitry 1645 is shown to the user via a display 1660. Additionally, the data collected/measured by the processor circuitry 1645 can be stored in a memory circuit 1680. Further, the processor circuitry 1645 can optionally control a haptic feedback circuit 1665, a speaker or buzzer 1670, a wired/wireless interface 1675, and an auxiliary sensor 1685 for one-way or two-way communication between the scale and the user.

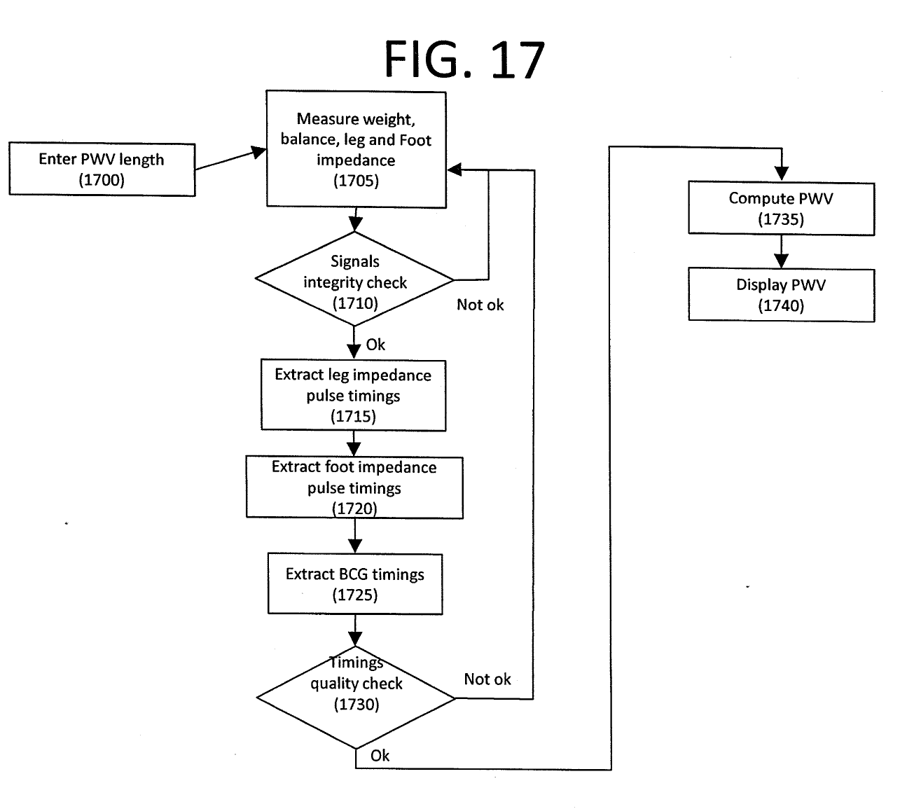

FIG. 17 shows an example flow diagram, consistent with various aspects of the present disclosure. At block 1700, a PWV length is entered. At block 1705, a user's weight, balance, leg, and foot impedance are measured. At 1710, the integrity of signals is checked (e.g., SNR). If the signal integrity check is not met, the user's weight, balance, leg, and foot impedance are measured again (block 1705), if the signals integrity check is met, the leg impedance pulse timings are extracted (as is shown at block 1715). At block 1720, foot impedance and pulse timings are extracted, and at block 1725, BCG timings are extracted. At block 1730, a timings quality check is performed. If the timings quality check is not validated, the user's weight, balance, leg and foot impedance are again measured (block 1705). If the timings quality check is validated, the PWV is calculated (as is shown at block 1735). At block 1740, the PWV is displayed to the user.

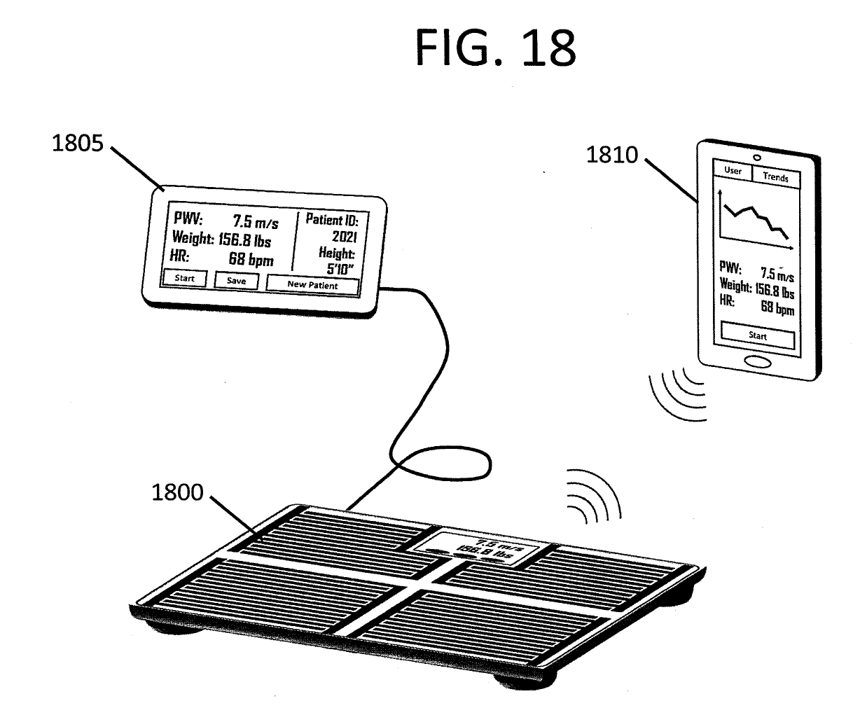

FIG. 18 shows an example scale 1800 communicatively coupled to a wireless device, consistent with various aspects of the present disclosure. As described herein, a display 1805 displays the various aspects measured by the scale 1800. The scale, in some embodiments, also wirelessly broadcast the measurements to a wireless device 1810. The wireless device 1810, in various embodiments, is implemented as an iPad®, smart phone or other CPU to provide input data for configuring and operating the scale.

As an alternative or complementary user interface, the scale includes a FUI which is enabled/implementable by one or more foot-based biometrics (for example, with the user being correlated to previously-entered user weight, toe print, an ECG-to-BCG timing relationship, and/or foot size/shape). The user foot-based biometric, in some embodiments, is implemented by the user manually entering data (e.g., a password) on the upper surface or display area of the scale. In implementations in which the scale is configured with a haptic, capacitive or flexible pressure-sensing upper surface, the (upper surface/tapping) touching from or by the user is sensed in the region of the surface and processed according to conventional X-Y grid Signal processing in the logic circuitry/CPU that is within the scale. By using one or more of the accelerometers located within the scale at its corners, such user data entry is sensed by each such accelerometer so long as the user's toe, heel or foot pressure associated with each tap provides sufficient force. Although the present discussion refers to a FUI, embodiments are not so limited. Various embodiments include internal or external GUIs that are in communication with the scale and used to obtain a biometric and that can be in place of the FUI and/or in combination with a FUI. For example, a user device having a GUI, such as tablet, is in communication with the scale via a wired or wireless connection. The user device obtains a biometric, such a finger print, and communicates the biometric to the scale.

In various embodiments, the above discussed user-interface is used with other features described herein for the purpose of storing and securing data that is sensitive to the data such as: the configuration data input by the user, the biometric and/or passwords entered by the user, and the user-specific health related data which might include data that is less sensitive to the user (e.g., the user's weight) and data that is more sensitive to the user (e.g., the user's scale obtains cardiograms and other data generated by or provided to the scale and associated with the user's symptoms and/or diagnoses). For such user data, the above described biometrics are used as directed by the user for indicating and defining protocol to permit such data to be exported from the scale to other remote devices indoor locations. In more specific embodiments, the scale operates in different modes of data security including, for example: a default mode in which the user's body mass and/or weight is displayed regardless of any biometric which would associate with the specific user standing on the scale; another mode in which complicated data (or data reviewed infrequently) is only exported from the scale under specific manual commands provided to the scale under specific protocols; and another mode or modes in which the user-specific data that is collected from the scale is processed and accessed based on the type of data. Such data categories include categories of different level of importance and/or sensitivities such as the above-discussed high and low level data and other data that might be very specific to a symptom and/or degrees of likelihood for diagnoses. Optionally, the CPU in the scale is also configured to provide encryption of various levels of sensitivity of the user's data.

For example, in accordance with various embodiments, the above-described FUI is used to provide portions of the clinical indications (e.g., scale-obtained physiological data) and/or additional health information to the user. In some embodiments, the scale includes a display configuration filter (e.g., circuitry and/or computer readable medium) configured to discern the data to display to the user and display portion. The display configuration filter discerns which portions of the clinical indications and/or additional health information to display to the user on the FUI based on various user demographic information (e.g., age, gender, height, diagnosis) and the amount of data. For example, the clinical indication may include an amount of data that if all the data is displayed on the FUI, the data is difficult for a person to read and/or uses multiple display screens.

The display configuration filter discerns portions of the data to display using the scale user interface, such as synopsis of the clinical indication (or additional health information) and an indication that additional data is displayed on another user device, and other portions to display on the other user device. The other user device is selected by the scale (e.g., the filter) based on various communications settings. The communication settings include settings such as user settings (e.g., the user identifying user devices to output data to), scale-based biometrics (e.g., user configures scale, or default settings, to output data to user devices in response to identifying scale-based biometrics), and/or proximity of the user device (e.g., the scale outputs data to the closest user device among a plurality of user devices and/or in response to the user device being within a threshold distance from the scale), among other settings. For example, the scale determines which portions of the clinical indication or additional health information to output and outputs the remaining portion of the clinical indication or additional health information to a particular user device based on user settings/communication authorization (e.g., what user devices are authorized by the user to receive particular user data from the scale), and proximity of the user device to the scale. The determination of which portions to output is based on what type of data is being displayed, how much data is available, and the various user demographic information (e.g., an eighteen year old is able to see better than a fifty year old).

For example, in some specific embodiments, the scale operates in different modes of data security and communication. The different modes of data security and communication are enabled in response to biometrics identified by the user and using the FUI. In some embodiments, the scale is used by multiple users and/or the scale operates in different modes of data security and communication in response to identifying the user and based on biometrics. The different modes of data security and communication include, for example: a first mode (e.g., default mode) in which the user's body mass and/or weight is displayed regardless of any biometric which would associate with the specific user standing on the scale and no data is communicated to external circuitry; a second mode in which complicated/more-sensitive data (or data reviewed infrequently) is only exported from the scale under specific manual commands provided to the scale under specific protocols and in response to a biometric; and third mode or modes in which the user-specific data that is collected from the scale is processed and accessed based on the type of data and in response to a biometric. Such data categories include categories of different levels of importance and/or sensitivities such as the above-discussed high and low level data and other data that might be very specific to a symptom and/or degrees of likelihood for diagnoses. Optionally, the CPU in the scale is also configured to provide encryption of various levels of sensitivity of the user's data.

In some embodiments, the different modes of data security and communication are enabled in response to recognizing the user standing on the scale using a biometric and operating in a particular mode of data security and communication based on user preferences and/or services activated. For example, the different modes of operation include the default mode (as discussed above) in which certain data (e.g., categories of interest, categories of user data, or historical user data) is not communicated from the scale to external circuitry, a first communication mode in which data is communicated to external circuitry as identified in a user profile, a second or more communication modes in which data is communicated to a different external circuitry for further processing. The different communication modes are enabled based on biometrics identified from the user and user settings in a user profile corresponding with each user.

In a specific embodiment, a first user of the scale may not be identified and/or have a user profile set up. In response to the first user standing on the scale, the scale operates in a default mode. During the default mode, the scale displays the user's body mass and/or weight on the user display and does not output user data. A second user of the scale has a user profile set up that indicates the user would like data communicated to a computing device of the user. When the second user stands on the scale, the scale recognizes the second user based on a biometric and operates in a first communication mode. During the first communication mode, the scale outputs at least a portion of the user data to an identified external circuitry. For example, the first communication mode allows the user to upload data from the scale to a user identified external circuitry (e.g., the computing device of the user). The information may include additional health information and/or user information that has low-user sensitivity. In the first communication mode, the scale performs the processing of the raw sensor data and/or the external circuitry can. For example, the scale sends the raw sensor data and/or additional health information to a user device of the user. The computing device may not provide access to the raw sensor data to the user and/or can send the raw sensor data to another external circuitry for further processing in response to a user input. For example, the computing device can ask the user if the user would like additional health information and/or regulated health information as a service. In response to receiving an indication the user would like the additional health information and/or regulated health information, the computing device outputs the raw sensor data and/or non-regulated health information to another external circuitry for processing, providing to a physician for review, and controlling access, as discussed above.

In one or more additional communication modes, the scale outputs raw sensor data to an external circuitry for further processing. For example, during a second communication mode and a third communication, the scale sends the raw sensor data and other data to external circuitry for processing, such as to a remote user-physiological device for correlation and processing. Using the above-provided example, a third user of the scale has a user profile set up that indicates the third user would like scale-obtained data to be communicated to a remote user-physiological device for further processing, such as to correlate the cardio-data sets and/or further process the correlated data sets. When the third user stands on the scale, the scale recognizes the third user based on one or more biometrics and operates in a second communication mode. During the second communication mode, the scale outputs the raw sensor data to the remote user-physiological device. The remote user-physiological device correlates the raw sensor data from the scale with cardio-physiological data from the remote user-physiological device, determines at least one physiological parameter of the user, and, optionally, derives additional health information. In some embodiments, the remote user-physiological device outputs data, such as the physiological parameter or additional health information to the scale. The scale, in some embodiments, displays a synopsis of the additional health information and outputs a full version of the additional health information to another user device for display (such as, using the filter described above) and/or an indication that additional health information can be accessed.

A fourth user of the scale has a user profile set up that indicates the fourth user has enabled a service to access regulated health information. When the fourth user stands on the scale, the scale recognizes the user based on one or more biometrics and operates in a fourth communication mode. In the fourth communication mode, the scale outputs raw sensor data to the external circuitry, and the external circuitry processes the raw sensor data and controls access to the data. For example, the external circuitry may not allow access to the regulated health information until a physician reviews the information. In some embodiments, the external circuitry outputs data to the scale, in response to physician review. For example, the output data can include the regulated health information and/or an indication that regulated health information is ready for review. The external circuitry may be accessed by the user, using the scale and/or another user device. In some embodiments, using the FUI of the scale, the scale displays the regulated health information to the user. The scale, in some embodiments, displays a synopsis of the regulated health information (e.g., clinical indication) and outputs the full version of regulated health information to another user device for display (such as, using the filter described above) and/or an indication that the regulated health information can be accessed to the scale to display. In various embodiments, if the scale is unable to identify a particular (high security) biometric that enables the fourth communication mode, the scale may operate in a different communication mode and may still recognize the user. For example, the scale may operate in a default communication mode in which the user data collected by the scale is stored in a user profile corresponding to the fourth user and on the scale. In some related embodiments, the user data is output to the external circuitry at a different time.

Although the present embodiments illustrates a number of security and communication modes, embodiments in accordance with the present disclosure can include additional or fewer modes. Furthermore, embodiments are not limited to different modes based on different users. For example, a single user may enable different communication modes in response to particular biometrics of the user identified and/or based on user settings in a user profile.

In various embodiments, the scale defines a user data table that defines types of user data and sensitivity values of each type of user data, as previously illustrated herein. In specific embodiments, the FUI displays the user data table. In other specific embodiments a user interface of a smartphone, tablet, and/or other computing device displays the user data table. For example, a wired or wireless tablet is used, in some embodiments, to display the user data table. The sensitivity values of each type of user data, in some embodiments, define in which communication mode(s) the data type is communicated and/or which biometric is used to enable communication of the data type. In some embodiments, a default or pre-set user data table is displayed and the user revises the user data table using the FUI. The revisions are in response to user inputs using the user's foot and/or contacting or moving relative to the FUI. Although the embodiments are not so limited, the above (and below) described control and display is provided using a wireless or wired tablet or other computing device as a user interface. The output to the wireless or wired tablet, as well as additional external circuitry, is enabled using biometrics. For example, the user is encouraged, in particular embodiments, to configure the scale with various biometrics. The biometric include scale-based biometrics and biometrics from the tablet or other user computing device. The biometric, in some embodiments, used to enable output of data to the tablet and/or other external circuitry includes a higher integrity biometric (e.g., higher likelihood of identifying the user accurately) than a biometric used to identify the user and stored data on the scale.

In various embodiments, the user adjusts the table displayed above to revise the sensitivity values of each data type. Further, although the above-illustrated table includes a single sensitivity value for each data type, in various embodiments, one or more of the data types are separated into sub-data types and each sub-data type has a sensitivity value. As an example, the user-specific advertisement is separated into: prescription advertisement, external device advertisements, exercise advertisements, and diet plan advertisement. Alternatively and/or in addition, the sub-data types for user-specific advertisement include generic advertisements based on a demographic of the user and advertisements in response to scale collected data (e.g., advertisement for a device in response to physiologic parameters), as discussed further herein.

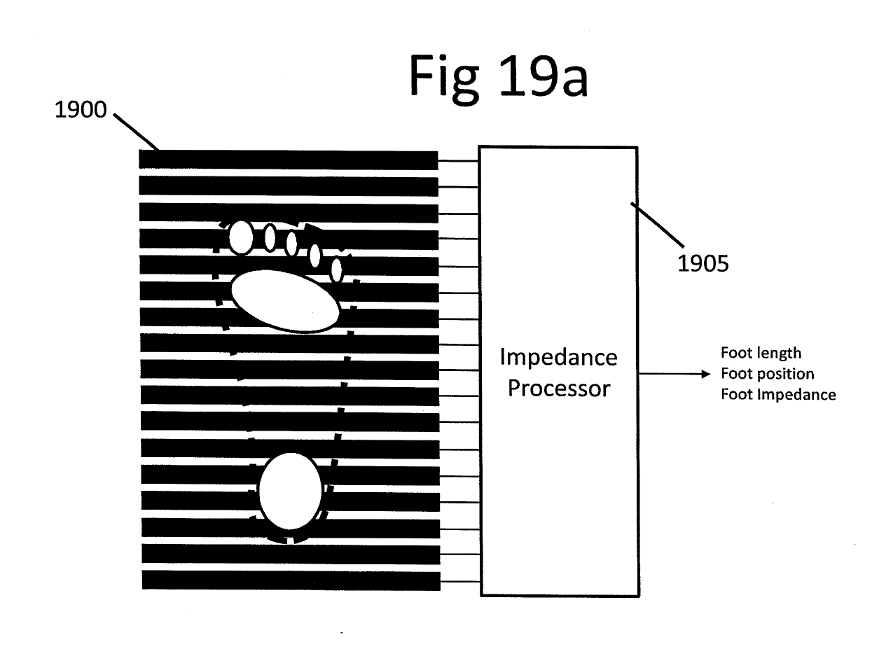

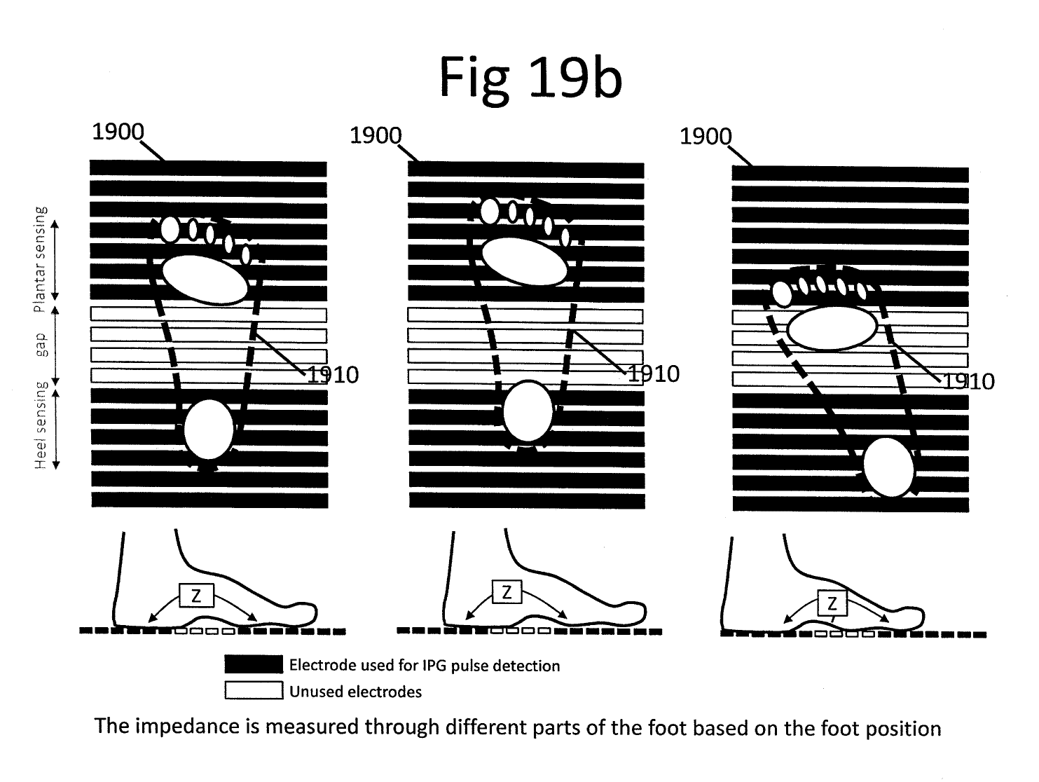

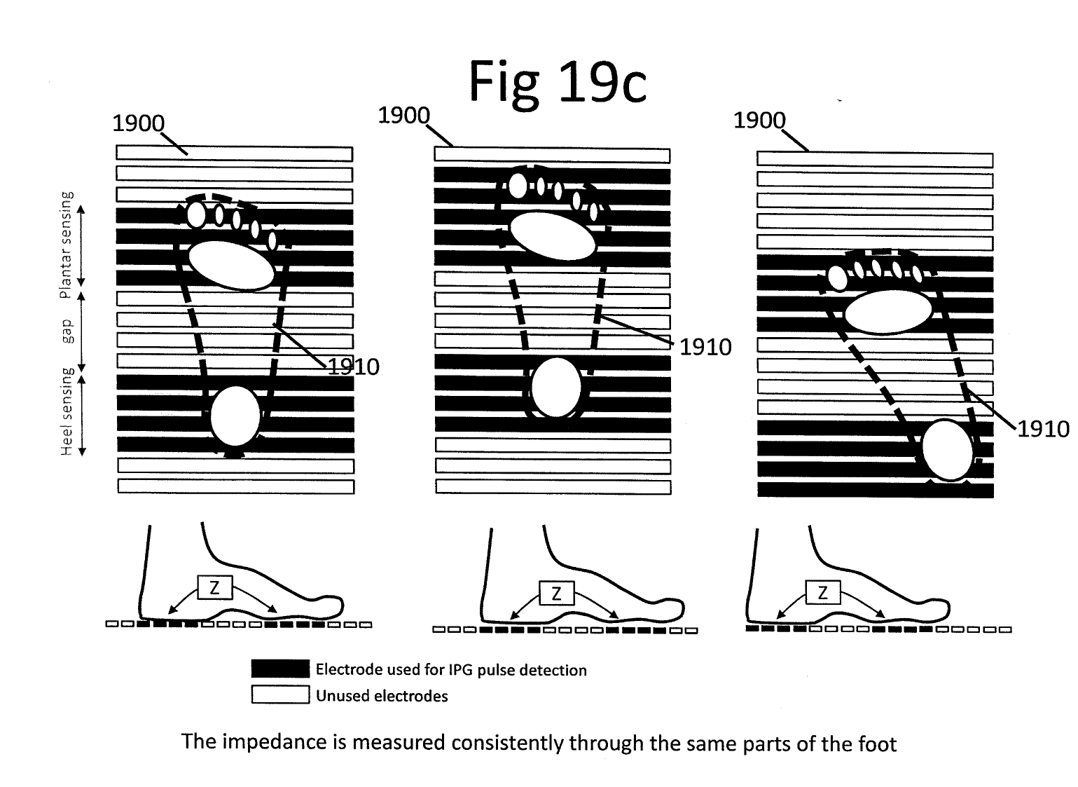

FIGS. 19a-c show example impedance as measured through different parts of the foot based on the foot position, consistent with various aspects of the present disclosure. For instance, example impedance measurement configurations may be implemented using a dynamic electrode configuration for measurement of foot impedance and related timings. Dynamic electrode configuration may be implemented using independently-configurable electrodes to optimize the impedance measurement. As shown in FIG. 19a, interleaved electrodes 1900 are connected to an impedance processor circuit 1905 to determine foot length, foot position, and/or foot impedance. As is shown in FIG. 19b, an impedance measurement is determined regardless of foot position 1910 based on measurement of the placement of the foot across the electrodes 1900. This is based in part in the electrodes 1900 that are engaged (blackened) and in contact with the foot (based on the foot position 1910), which is shown in FIG. 19c.

More specifically regarding FIG. 19a, configuration includes connection/de-connection of the individual electrodes 1900 to the impedance processor circuit 1905, their configuration as current-carrying electrodes (injection or return), sense electrodes (positive or negative), or both. The configuration is preset based on user information, or updated at each measurement (dynamic reconfiguration) to optimize a given parameter (impedance SNR, measurement location). The system algorithmically determines which electrodes under the foot to use in order to obtain the highest SNR in the pulse impedance signal. Such optimization algorithm may include iteratively switching configurations and measuring the impedance, and selecting the best suited configuration. Alternatively, the system first, through a sequential impedance measurement between each individual electrode 1900 and another electrode in contact with the body (such as an electrode in electrode pair 205 on the other foot), determine which electrodes are in contact with the foot. By determining the two most apart electrodes, the foot size is determined. Heel location can be determined in this manner, as can other characteristics such as foot arch type. These parameters are used to determine programmatically (in an automated manner by CPU/logic circuitry) which electrodes are selected for current injection and return (and sensing if a Kelvin connection issued) to obtain the best foot IPG.

In various embodiments involving the dynamically reconfigurable electrode array 1900/1905, an electrode array set is selected to measure the same portion/segment of the foot, irrespective of the foot location on the array. FIG. 19b illustrates the case of several foot positions on a static array (a fixed set of electrodes are used for measurement at the heel and plantar/toe areas, with a fixed gap of an inactive electrode or insulating material between them). Depending on the position of the foot, the active electrodes are contacting the foot at different locations, thereby sensing a different volume/segment of the foot. If the IPG is used by itself (e.g., for heart measurement), such discrepancies may be non-consequential. However, if timings derived from the IPG are referred to other timings (e.g., R-wave from the ECG, or specific timing in the BCG), such as for the calculation of a PTT or PWV, the small shifts in IPG timings due to the sensing of slightly different volumes in the foot (e.g., if the foot is not always placed at the same position on the electrodes) can introduce an error in the calculation of the interval. With respect to FIG. 19b, the timing of the peak of the IPG from the foot placement on the right (sensing the toe/plantar region) is later than from the foot placement on the left, which senses more of the heel volume (the pulse reaches first the heel, then the plantar region). Factors influencing the magnitude of these discrepancies include foot shape (flat or not) and foot length.

Various embodiments address challenges relating to foot placement. FIG. 19c shows an example embodiment involving dynamic reconfiguration of the electrodes to reduce such foot placement-induced variations. As an example, by sensing the location of the heel first (as described above), it is possible to activate a subset of electrodes under the heel, and another subset of electrodes separated by a fixed distance (1900). The other electrodes (e.g., unused electrodes) are left disconnected. The sensed volume will therefore be the same, producing consistent timings. The electrode configuration leading to the most consistent results may be informed by the foot impedance, foot length, the type of arch (all of which can be measured by the electrode array as shown above), but also by the user ID (foot information can be stored for each user, then looked up based on automatic user recognition or manual selection (e.g., in a look-up-table stored for each user in a memory circuit accessible by the CPU circuit in the scale).

In certain embodiments, the apparatus measures impedance using a plurality of electrodes contacting one foot and with at least one other electrode (typically many) at a location distal from the foot. The plurality of electrodes (contacting the one foot) is arranged on the platform and in a pattern configured to inject current signals and sense signals in response thereto, for the same segment of the foot so that the timing of the pulse-based measurements does not vary because the user placed the one foot at a slightly different position on the platform or scale. In FIG. 19a, the foot-to-electrode locations for the heel are different locations than that shown in FIGS. 19b and 19c. As this different foot placement can occur from day to day for the user, the timing and related impedance measurements are for the same (internal) segment of the foot. By having the processor circuit inject current and sense responsive signals to first locate the foot on the electrodes (e.g., sensing where positions of the foot's heel plantar regions and/or toes), the pattern of foot-to-electrode locations permits the foot to move laterally, horizontally and both laterally and horizontally via the different electrode locations, while collecting impedance measurements relative to the same segment of the foot.

The BCG/IPG system can be used to determine the PTT of the user, by identification of the average I-Wave or derivative timing near the I-Wave from a plurality of BCG heartbeat signals obtained simultaneously with the Dual-IPG measurements of the present disclosure to determine the relative PTT along an arterial segment between the ascending aortic arch and distal pulse timing of the user's lower extremity. In certain embodiments, the BCG/IPG system is used to determine the PWV of the user, by identification of the characteristic length representing the length of the user's arteries, and by identification of the average I-Wave or derivative timing near the I-Wave from a plurality of BCG heartbeat signals obtained simultaneously with the Dual-IPG measurements of the present disclosure to determine the relative PTT along an arterial segment between the ascending aortic arch and distal pulse timing of the user's lower extremity. The system of the present disclosure and alternate embodiments may be suitable for determining the arterial stiffness (or arterial compliance) and/or cardiovascular risk of the user regardless of the position of the user's feet within the bounds of the interleaved electrodes. In certain embodiments, the weighing scale system incorporated the use of strain gage load cells and six or eight electrodes to measure a plurality of signals including: bodyweight, BCG, body mass index, fat percentage, muscle mass percentage, and body water percentage, heart rate, heart rate variability, PTT, and PWV measured simultaneously or synchronously when the user stands on the scale to provide a comprehensive analysis of the health and wellness of the user.

In other certain embodiments, the PTT and PWV are computed using timings from the Leg IPG or Foot IPG for arrival times, and using timings from a sensor located on the upper body (as opposed to the scale measuring the BCG) to detect the start of the pulse. Such sensor may include an impedance sensor for impedance cardiography, a hand-to-hand impedance sensor, a photoplethysmogram on the chest, neck, head, arms or hands, or an accelerometer on the chest (seismocardiograph) or head.

Communication of the biometric information is another aspect of the present disclosure. The biometric results from the user are stored in the memory on the scale and displayed to the user via a display on the scale, audible communication from the scale, and/or the data is communicated to a peripheral device such as a computer, smart phone, tablet computing device. The communication occurs to the peripheral device with a wired connection, or can be sent to the peripheral device through wireless communication protocols such as Bluetooth or WiFi. Computations such as signal analyses described therein may be carried out locally on the scale, in a smartphone or computer, or in a remote processor (cloud computing).

Other aspects of the present disclosure are directed toward apparatuses or methods that include the use of at least two electrodes that contacts feet of a user. Further, circuitry is provided to determine a pulse arrival time at the foot based on the recording of two or more impedance signals from the set of electrodes. Additionally, a second set of circuitry is provided to extract a first pulse arrival time from a first impedance signal and use the first pulse arrival time as a timing reference to extract and process a second pulse arrival time in a second impedance signal.

Various embodiments are implemented in accordance with, and fully incorporating by reference for their general teachings, the above-identified PCT Applications and U.S. Provisional Applications (including PCT Ser. No. PCT/US2016/062484 and PCT Ser. No. PCT/US2016/062505), which teachings are also incorporated by reference specifically concerning physiological scales and related measurements and communications such as exemplified by disclosure in connection with FIGS. 1a, 1b, 1e, 1f, 1k, 1l, 1m, 1n, and 2b-2e in PCT Ser. No. PCT/US2016/062484 and FIGS. 1a, 1k, 1l, and 1m in PCT. Ser. No. PCT/US2016/062505, and related disclosure in the above-identified U.S. Provisional Applications. For example, above-identified U.S. Provisional Application (Ser. No. 62/258,238), which teachings are also incorporated by reference specifically concerning obtaining derivation data, assessing a condition or treatment of the user, and drug titration features and aspects as exemplified by disclosure in connection with FIGS. 1a-1b of the underlying provisional; U.S. Provisional Application (Ser. No. 62/266,496), which teachings are also incorporated by reference specifically to trigger data on the scale to trigger a filter of data on the enterprise system and identify correlated data with a user condition related to the trigger data features and aspects as described in connection with FIGS. 1a-1d in the underlying provisional; and U.S. Provisional Application (Ser. No. 62/266,523), which teachings are also incorporated by reference specifically concerning grouping users into inter and intra scale social groups based on aggregated user data sets, and providing normalized user data to other users in the social group aspects as exemplified by disclosure in connection with FIGS. 1a-1c of the underlying provisional. For instance, embodiments herein and/or in the PCT and/or provisional applications may be combined in varying degrees (including wholly). Reference may also be made to the experimental teachings and underlying references provided in the PCT and/or provisional applications. Embodiments discussed in the provisional applicants are not intended, in any way, to be limiting to the overall technical disclosure, or to any part of the claimed invention unless specifically noted.

Reference may also be made to published patent documents U.S. Patent Publication 2010/0094147 and U.S. Patent Publication 2013/0310700, which are, together with the references cited therein, herein fully incorporated by reference for the purposes of sensors and sensing technology. The aspects discussed therein may be implemented in connection with one or more of embodiments and implementations of the present disclosure (as well as with those shown in the figures). In view of the description herein, those skilled in the art will recognize that many changes may be made thereto without departing from the spirit and scope of the present disclosure.

As illustrated herein, various circuit-based building blocks and/or modules may be implemented to carry out one or more of the operations/activities described herein shown in the block-diagram-type figures. In such contexts, these building blocks and/or modules represent circuits that carry out these or related operations/activities. For example, in certain embodiments discussed above (such as the pulse circuitry modularized as shown in FIGS. 3a-b), one or more blocks/modules are discrete logic circuits or programmable logic circuits for implementing these operations/activities, as in the circuit blocks/modules shown. In certain embodiments, the programmable circuit is one or more computer circuits programmed to execute a set (or sets) of instructions (and/or configuration data). The instructions (and/or configuration data) can be in the form of firmware or software stored in and accessible from a memory circuit. As an example, first and second modules/blocks include a combination of a CPU hardware-based circuit and a set of instructions in the form of firmware, where the first module/block includes a first CPU hardware circuit with one set of instructions and the second module/block includes a second CPU hardware circuit with another set of instructions.

Based upon the above discussion and illustrations, those skilled in the art will readily recognize that various modifications and changes may be made to the present disclosure without strictly following the exemplary embodiments and applications illustrated and described herein. For example, the input terminals as shown and discussed may be replaced with terminals of different arrangements, and different types and numbers of input configurations (e.g., involving different types of input circuits and related connectivity). Such modifications do not depart from the true spirit and scope of the present disclosure, including that set forth in the following claims.

What is claimed is:

1. An apparatus comprising:
a weighing scale including:
a platform in which a plurality of electrodes and force sensor circuitry are integrated and configured and arranged for engaging a user; and
processing circuitry, including a CPU and a memory circuit with user-corresponding data stored in the memory circuit, configured and arranged with the plurality of electrodes and the force sensor circuitry to:
collect physiological data from the user while the user is standing on the platform;
identify the user based on an identified scale-based biometric within the physiological data;
identify a risk that the user has a health condition using trigger data indicative of risks for a plurality of health conditions, wherein the trigger data includes values of the physiological data that are indicative of the risks for the plurality of health conditions; and
output at least portions of the physiological data as user data in response to the identified risk; and
external circuitry, including processing circuitry and memory circuitry, configured and arranged to receive the user data from the scale, and in response:
filter data from the Internet using the user data; and
identify data related to the health condition based on the filter of the data from the Internet.

2. The apparatus of claim 1, wherein the external circuitry is configured and arranged to identify and provide the data related to the health condition to the scale.

3. The apparatus of claim 1, wherein the processing circuitry of the weighing scale is further configured and arranged to filter the physiological data using the trigger data and output the at least portion of the physiological data as user data, wherein the user data is indicative of the risk for the health condition.

4. The apparatus of claim 1, wherein the processing circuitry of the external is further configured and arranged to filter the user data based on the trigger data indicative of the health condition, and filter the Internet based on the filtered user data and the trigger data, wherein the filtered user data is indicative of the risk for the health condition.

5. The apparatus of claim 1, wherein the apparatus further includes a plurality of weighing scale, including the weighing scale, that communicate user data to the external circuitry.

6. The apparatus of claim 1, wherein the processing circuitry is configured and arranged to identify the risk that the user has the health condition by comparing the physiological data to the trigger data, wherein the trigger data includes the values of the physiologic data that indicate the user has a likelihood above threshold of having the health condition.

7. The apparatus of claim 6, wherein the scale further include a foot-controlled user interface (FUI) configured to provide data to the user and to receive inputs from the user's foot, wherein responsive to a match of the physiological data to the trigger data, the processing circuitry and the FUI are configured and arranged to prompt the user for additional health information and verify that the user is interested in the additional health information corresponding to the health condition responsive to a user foot-based input to the FUI.

8. The apparatus of claim 1, wherein the external circuitry is configured and arranged to filter data of the Internet for data correlating with the health condition and the trigger data including generic health information, lifestyle suggests, risks factors, and a combination thereof.

9. The apparatus of claim 1, wherein the external circuitry is configured and arranged to filter data of the Internet for data correlating with the health condition and the trigger data including additional tests and/or questions to ask the user using the scale.

10. The apparatus of claim 9, wherein the external circuitry is configured and arranged to output the additional tests and/or questions to the scale, wherein the processing circuitry of the scale is configured and arranged to perform the additional tests and/or output the questions to the user, wherein the output circuitry of the scale is configured and arranged to output answers to the questions to the external circuitry

11. The apparatus of claim 1, wherein the external circuitry is configured and arranged to identify additional risk factors for the health condition based on the filter of the Internet and output the additional risks as revised trigger data to the scale, wherein the processing circuitry of the scale is modified to include the revised trigger data.

12. The apparatus of claim 1, wherein the external circuitry is configured and arranged to provide a physician with access to the user data in response to a user input to the scale that activates physician review and output diagnosis data to the scale responsive to physician review.

13. An apparatus comprising:
a weighing scale including:
a platform in which a plurality of electrodes and force sensor circuitry are integrated and configured and arranged for engaging a user;
processing circuitry, including a CPU and a memory circuit with user-corresponding data stored in the memory circuit, configured and arranged under the platform upon which the user stands, the processing circuitry being electrically integrated with the plurality of electrodes and the force sensor circuitry and being configured to:
collect physiological data from the user while the user is standing on the platform;
identify the user based on an identified scale-based biometric within the physiological data;
identify a risk that the user has a health condition using trigger data, wherein the trigger data includes values of the physiological data that are indicative of risks for the plurality of health conditions;
filter the physiological data based on portions of the trigger data indicative of the health condition, wherein the filtered physiological data is indicative of the risk for the health condition;
output at least the filtered physiological data as user data in response to the identified risk and an indication of interest in additional health information related to the health condition; and
external circuitry, including processing circuitry and memory circuitry, configured and arranged to receive the user data from the scale, and in response:
filter data from the Internet using the user data and based on the trigger data indicative of the health risk; and
identify data related to the health condition based on the filter of the data from the Internet.

14. The apparatus of claim 13, wherein the external circuitry is configured and arranged to identify new correlations or risk factors for the health condition, additional devices and/or parameters, and output the identified new correlations, additional devices, and/or parameters to the scale, wherein the processing circuitry of the scale stores the identified new correlations, additional devices, and/or parameters.
